SEQUENCE LISTING (8 sequences)

SEQ ID NO: 1 - DNA, 18795 bp, Artificial Sequence
  Synthetic circular DNA construct suitable for Bacillus subtilis
  rep_origin (1)..(546): p15A ori
  promoter (549)..(990): pGAL1
  5′UTR (991)..(1015): 5′UTR 12 (doi 10.1021/acssynbio.9b00355)
  sig_peptide (1016)..(1114): AmyE sig.pept (doi10.1128/mmbr.64.3.515-547.2000)
  enhancer (1115)..(1141): propeptide (doi 10.5772/29256)
  gene (1142)..(3097): AHAS1 (Q5VB45_HELAN) doi 10.1002/bit.10728
  sig_peptide (3098)..(3199): sortase cleavage signal (10.1128/JB.186.16.5376-5383.2004)
  3′UTR (3203)..(3222): 3′UTR (doi 10.1093/bioinformatics/15.7.578)
  terminator (3223)..(3261): Terminator BBa_B1006
  CDS (3262)..(4140): araC, L-arabinose regulatory protein
  promoter (4167)..(4451): araBAD, promoter of the L-arabinose operon of E. coli
  primer_bind (4374)..(4393): pBAD Forward
  5′UTR (4452)..(4476): 5′UTR 12 (doi 10.1021/acssynbio.9b00355)(8)
  sig_peptide (4477)..(4575): AmyE sig.pept (doi10.1128/mmbr.64.3.515-547.2000)(4)
  enhancer (4576)..(4602): propeptide (doi 10.5772/29256)(4)
  gene (4603)..(5985): Transaminase E1V913_HALED (EC 2.6.1)
  sig_peptide (5986)..(6087): sortase cleavage signal (10.1128/JB.186.16.5376-5383.2004)(4)
  3′UTR (6091)..(6110): 3′UTR (doi10.1093/bioinformatics/15.7.578)(8)
  terminator (6111)..(6149): Terminator BBa_B1006(8)
  promoter (6150)..(6627): alcA promoter (GB KC189906.1)
  5′UTR (6628)..(6652): 5′UTR 12 (doi 10.1021/acssynbio.9b00355)(1)
  sig_peptide (6653)..(6751): AmyE sig.pept (doi10.1128/mmbr.64.3.515-547.2000)(1)
  enhancer (6752)..(6778): propeptide (doi 10.5772/29256)(1)
  gene (6779)..(7009): Salsolinol synthase (doi10.1021/acschemneuro.8b00023)
  sig_peptide (7010)..(7111): sortase cleavage signal (10.1128/JB.186.16.5376-5383.2004)(1)
  3′UTR (7115)..(7134): 3′UTR (doi10.1093/bioinformatics/15.7.578)(2)
  terminator (7135)..(7173): Terminator BBa_B1006(2)
  promoter (7174)..(7229): p43 promoter (iGEM BBa_K143013)
  5′UTR (7230)..(7254): 5′UTR 12 (doi 10.1021/acssynbio.9b00355)(7)
  gene (7255)..(9720): alcR
  3′UTR (9721)..(9740): 3′UTR (doi10.1093/bioinformatics/15.7.578)(1)
  terminator (9741)..(9779): Terminator BBa_B1006(1)
  promoter (9780)..(9965): CUP1 promoter
  5′UTR (9966)..(9990): 5′UTR 12 (doi 10.1021/acssynbio.9b00355)(2)
  sig_peptide (9991)..(10089): AmyE sig.pept (doi10.1128/mmbr.64.3.515-547.2000)(2)
  enhancer (10090)..(10116): propeptide (doi 10.5772/29256)(2)
  gene (10117)..(10689): Norcolaurine synthase (EC 4.2.1.78, DOI 10.1039/c7cc08024g) A79I
  sig_peptide (10690)..(10791): sortase cleavage signal (10.1128/JB.186.16.5376-5383.2004)(3)
  3′UTR (10795)..(10814): 3′UTR (doi10.1093/bioinformatics/15.7.578)(5)
  terminator (10815)..(10853): Terminator BBa_B1006(5)
  promoter (10854)..(10950): Pveg promoter (igem BBa_K143012)
  5′UTR (10951)..(10975): 5′UTR 12 (doi 10.1021/acssynbio.9b00355)(5)
  gene (10976)..(11650): CUP2 regulator (GB DAA07947.1 doi 10.1128/mcb.9.9.4091)
  3′UTR (11654)..(11673): 3′UTR (doi10.1093/bioinformatics/15.7.578)(6)
  terminator (11674)..(11712): Terminator BBa_B1006(6)
  primer_bind (11713)..(11745): FerFOW1
  promoter (11728)..(14632): F0-Fer Promoter (doi10.1186/s13071-019-3574-9)
  primer_bind (14614)..(14632): FerREV1
  5′UTR (14633)..(14657): 5′UTR 12 (doi 10.1021/acssynbio.9b00355)(4)
  sig_peptide (14658)..(14756): AmyE sig. pept (doi10.1128/mmbr.64.3.515-547.2000)(3)
  enhancer (14757)..(14783): propeptide (doi 10.5772/29256)(3)
  gene (14784)..(15335): P11086 PNMT (EC 2.1.1.28)
  sig_peptide (15336)..(15437): sortase cleavage signal (10.1128/JB.186.16.5376-5383.2004)(2)
  3′UTR (15441)..(15460): 3′UTR (doi10.1093/bioinformatics/15.7.578)(3)
  terminator (15461)..(15499): Terminator BBa_B1006(3)
  misc_feature (15500)..(16504): RepB
  rep_origin (16657)..(16683): OriU(https//www.ncbi.nlm.nih.gov/nuccore/U3236 9.1)
  promoter (17001)..(17036): Bacillus phage SPO1 promoter (NC_011421.1,iGEM BBa_K780003)
  5′UTR (17037)..(17061): 5′UTR 12 (doi 10.1021/acssynbio.9b00355)(6)
  misc_feature (17062)..(17742): mRaspberry reporter ex 598nm
  3′UTR (17743)..(17762): 3′UTR (doi10.1093/bioinformatics/15.7.578)(7)
  terminator (17763)..(17801): Terminator BBa_B1006(7)
  promoter (17802)..(17850): pBL9 promoter (doi10.1007/s10529-017-2304-7)
  5′UTR (17851)..(17875): 5′UTR 12 (doi 10.1021/acssynbio.9b00355)(3)
  sig_peptide (17876)..(17935): Signalling peptide PrsA (WP_015383240.1)
  misc_feature (17936)..(18733): PrsABl-Bn (doi 10.1186/s12934-019-1203-0)
  3′UTR (18737)..(18756): 3′UTR (doi10.1093/bioinformatics/15.7.578)(4)
  terminator (18757)..(18795): Terminator BBa_B1006(4)
ttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggtatattagaaaggaggaatatataatgtttgcaaaaagatttaaaacatcactgctgccgctgtttgcaggctttctgctgctgtttcatctggttctggcaggcccggcagcagcatcagcactggaaatttcatcaacatgcgatgcaatggccgctccacctaacccaagtatctcgttcaaaccgccttctccggcggctgcgttacctcctcgttcagcgtttcttccgcgctttgccttgccaatcacgtccacaactcagaaaaggcaccgcttgcatatcagcaatgtcttgagtgacagtaaatccaccacaacaaccacacaaccgcctctgcaagcccaaccgtttgtcagcagatatgctccggatcaacctcggaaaggagccgacgttctcgtcgaagcgctggaacgtgaaggagttacagacgtgtttgcgtatccgggaggggcttctatggaaatccatcaggcattaacccgcagcaacacgattcggaatgttttacctcgtcatgaacagggaggagtctttgcagcagagggatatgcgagagcgtcaggactccccggcgtatgcattgcaacttctggccctggagctacgaacttagtgtctggtcttgcagatgcgctgctggactctgttccgatggtagcgattacgggtcaagtcccaagacggatgattggcactgatgtgttccaagaaacacccatcgtggaagtgaccagatccatcacaaagcacaattatctggtcttagatgttgaggatataccgaggattgtacgcgaagcattttatcttgcctcgtcagggcgtcctggccctgtcttgattgatgttcctaaagacatccaacagcaactggtggtcccgaaatgggatgagccgatgcggcttcccggatacttatcacggatgccaaaaccgcaatatgatggccatctggaacagatcgttcgcttagtaggtgaagctaagcggccggttctgtacgtaggcggtgggtgtctgaattcagatgatgagctgagaagatttgtggaactcacaggcataccagttgctagcacgttgatgggattgggagcgtatccggcgtcatcagatctgtctcttcatatgctggggatgcatggcacagtgtacgccaactatgctgttgacaaatctgatttactcttggcatttggcgttcgttttgatgacagagttactggcaaacttgaagcgtttgcgagccgagccaaaattgtccacatagacatcgatcctgccgagattgggaagaacaaacaaccgcatgtgtcaatttgcggggacatcaaagttgcacttcaaggacttaacaaaatccttgaggaaaagaattcggtaacaaaccttgatttcagcaattggcgcaaggaactggatgaacagaaagtgaaatttccgcttagcttcaagacgtttggtgaagcaatcccaccgcagcatgccatacaggtgttggatgagcttacaggtggcaatgcgattatttcgacaggtgttggtcaacatcagatgtgggcagctcaattttataagtacaacaaacctcgacagtggctcacgagtggaggtttaggcgcaatggggtttggtctgccagctgccattggcgctgcggttgctagaccggatgcggtcgtcgtcgacatcgatggagatggatccttcatgatgaatgtacaggaattagcaacgatacgtgttgagaatttgccggtcaaaatccttctgctcaataaccagcacttaggaatggtagtacagtgggaagatcgcttctacaaagctaatcgagctcatacctaccttggcaatccgtccaaagagagcgagattttcccaaacatggtcaaatttgccgaagcatgtgacattccggctgcacgagtgactcaaaaggccgatttaagggccgcaattcagaaaatgctggatacaccgggcccctatctcctggacgtgattgtgcctcatcaagaacacgtattgcccatgattccagcaggaggcggcttttcagacgtaattaccgaaggcgatgggagaacgaagtatctgccggatacatcagcaggctattataatgaaatggttattggcgcagcagttacaatttcaggcacatatctggttagaagaaaaagagcatcaagaacataaggctggatcacctcctttctaaaaaaaaaccccgcccctgacagggcggggttttttttttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccgttcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatgtatattagaaaggaggaatatataatgtttgcaaaaagatttaaaacatcactgctgccgctgtttgcaggctttctgctgctgtttcatctggttctggcaggcccggcagcagcatcagcactggaaatttcatcaacatgcgatgcaatgcaaacacaggactatcaggctttagaccgagcacatcatcttcacccttttacggatttcaaagcccttggggaagaagggtccagagtcgtcacccatgcagaaggcgtatatatccacgattcagagggtaatcgcattttggatgggatggctggactctggtgtgtgaacctcggatatggccgtagagaactcgtagaagccgcaacggctcaattggaacagcttccgtactataacaccttctttaaaacgacgcatccaccagctgtgcgtcttgcggagaagctgtgcgatttagcaccggctcacattaatcgcgtgttctttaccggttccgggagtgaagcgaacgataccgtattacgtatggtcaggcgctattgggcccttaaaggacaaccggataaacagtggattatcggacgcgagaatgcatatcatgggagcacattagcgggtatgagccttggaggtatggcacctatgcacgctcaaggcggaccatgtgttcctggcattgcccatatccgtcaaccgtactggtttggagaaggtcgagatatgtcaccggaggcgtttggccagacatgtgccgaagcacttgaagagaaaatcttggaactcggcgaggagaaagttgccgcgtttatagctgaaccagttcaaggtgcaggaggtgcaattatgcctccggaaagctactggcctgccgtcaagaaggtgctggctaaatacgacattttgcttgtagcggacgaagttatttgcggatttggacggttaggagaatggtttggctctcagcactatggattagaaccggatctgatgcccatagcgaaaggactttcgtcaggctatttgccgattggcggagtccttgttggcgatagagttgcggagacactgatcgaggagggcggtgaattctttcatggctttacgtactcgggccatccgacatgcgctgctgtcgcgttaaagaaccttgaactgctggaagctgaaggggtagtggatcgcgtgagagatgatctgggcccttatctcgccgaaaggtgggcctctctggtagatcatcccatcgttggtgaagctaggagtctgggattgatgggcgcattggaattagtcgcggacaaaactactggccaacggtttgacaaatcattgggcgcagggaatctgtgtcgggatctgtgcttcgcgaatgggctggttatgcgttctgtgggagatacgatgatcattagcccgccactggtgattcgaagagaagagatagacgaactcgtcgagttagccagacgggcattagacgaaacagcgagacagctgacacaagttccgcatacacaggaagagcctactgcactgccggatacatcagcaggctattataatgaaatggttattggcgcagcagttacaatttcaggcacatatctggttagaagaaaaagagcatcaagaacataaggctggatcacctcctttctaaaaaaaaaccccgcccctgacagggcggggttttttttatgcatgcggaaccgcacgaggatgcatgcggaaccgcacgaggatgcatgcggaaccgcacgaggatgcatgcggaaccgcacgaggatgcatgcggaaccgcacgaggtcgatagttgtgatagttcccacttgtccgtccgcatcggcatccgcagctcgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaacagggatgacgcacaatcccactatccttcgcaagacccttcctctatataaggaagttcatttcatttggagaggacctcggtatattagaaaggaggaatatataatgtttgcaaaaagatttaaaacatcactgctgccgctgtttgcaggctttctgctgctgtttcatctggttctggcaggcccggcagcagcatcagcactggaaatttcatcaacatgcgatgcaatgcagattttcgtcaaaacacttacagggaaaacgataacactggaagtagaaccgtctgataccatcaagaatgtgaaagcgaagattcaggataaagagggtattcctccagatcaacaacggttgatctttgcaggcaaacaactcgaagatggaagaactctgtcagactacaacatccagaagaaaagcacgttacatctggttcttcgcttacgtgttgactatctgccggatacatcagcaggctattataatgaaatggttattggcgcagcagttacaatttcaggcacatatctggttagaagaaaaagagcatcaagaacataaggctggatcacctcctttctaaaaaaaaaccccgcccctgacagggcggggttttttttattttacatttttagaaatgggcgtgaaaaaaagcgcgcgattatgtaaaatataagtatattagaaaggaggaatatataatggcagatacaagaagaagacaaaatcattcatgcgatccgtgcagaaaaggcaaaagaagatgcgatgcaccggaaaatagaaatgaagcaaatgaaaatggctgggtttcatgctcaaattgcaaaagatggaataaagattgcacatttaattggctgtcatcacaaagatcaaaagcaaaaggcgcagcaccgagagcaagaacaaaaaaagcaagaacagcaacaacaacatcagaaccgtcaacatcagcagcaacaattccgacaccggaatcagataatcatgatgcaccgccggttattaattcacatgatgcactgccgtcatggacacaaggcctgctgtcacatccgggcgatctgtttgatttttcacattcagcaattccggcaaatgcagaagatgcagcaaatgttcaatcagatgcaccgtttccgtgggatctggcaattccgggcgatttttcaatgggccaacaactggaaaaaccgctgtcaccgctgtcatttcaagcagttctgctgccgccgcattcaccgaatacagatgatctgattagagaactggaagaacaaacaacagatccggattcagttacagatacaaattcagttcaacaagttgcacaagatggctcactgtggtcagatagacaatcaccgctgctgccggaaaattcactgtgcatggcatcagattcaacagcaagaagatatgcaagatcaacaatgacaaaaaatctgatgagaatttatcatgattcaatggaaaatgcactgtcatgctggctgacagaacataattgcccgtattcagatcaaatttcatatctgccgccgaaacaaagagcagaatggggcccgaattggtcaaatagaatgtgcattagagtttgcagactggatagagtttcaacatcactgagaggcagagcactgtcagcagaagaagataaagcagcagcaagagcactgcatctggcaattgttgcatttgcatcacaatggacacaacatgcacaaagaggcgcaggcctgaatgttccggcagatattgcagcagatgaaagatcaattagaagaaatgcatggaatgaagcaagacatgcactgcaacatacaacaggcattccgtcatttagagttatttttgcaaatattattttttcactgacacaatcagttctggatgatgatgaacaacatggcatgggcgcaagactggataaactgctggaaaatgatggcgcaccggtttttctggaaacagcaaatagacaactgtatacatttagacataaatttgcaagaatgcaaagaagaggcaaagcatttaatagactgccgggcggctcagttgcatcaacatttgcaggcatttttgaaacaccgacaccgtcatcagaatcaccgcaactggatccggttgttgcatcagaagaacatagatcaacactgtcactgatgttttggctgggcattatgtttgatacactgtcagcagcaatgtatcaaagaccgctggttgtttcagatgaagattcacaaatttcatcagcatcaccgccgagaagaggcgcagaaacaccgattaatctggattgctgggaaccgccgagacaagttccgtcaaatcaagaaaaatcagatgtttggggcgatctgtttctgagaacatcagattcactgccggatcatgaatcacatacacaaatttcacaaccggcagcaagatggccgtgcacatatgaacaagcagcagcagcactgtcatcagcaacaccggttaaagttctgctgtatagaagagttacacaactgcaaacactgctgtatagaggcgcatcaccggcaagactggaagcagcaattcaaagaacactgtatgtttataatcattggacagcaaaatatcaaccgtttatgcaagattgcgttgcaaatcatgaactgctgccgtcaagaattcaatcatggtatgttattctggatggccattggcatctggcagcaatgctgctggcagatgttctggaatcaattgatagagattcatattcagatattaatcatattgatctggttacaaaactgagactggataatgcactggcagtttcagcactggcaagatcatcactgagaggccaagaactggatccgggcaaagcatcaccgatgtatagacattttcatgattcactgacagaagttgcatttctggttgaaccgtggacagttgttctgattcattcatttgcaaaagcagcatatattctgctggattgcctggatctggatggccaaggcaatgcactggcaggctatctgcaactgagacaaaattgcaattattgcattagagcactgcaatttctgggcagaaaatcagatatggcagcactggttgcaaaagatctggaaagaggcctgaatggcaaagttgattcatttctgtaaggctggatcacctcctttctaaaaaaaaaccccgcccctgacagggcggggttttttttatgttcagcgaattaattaacttccaaaatgaaggtcatgagtgccaatgccaatgtggtagctgcaaaaataatgaacaatgccaaaaatcatgtagctgcccaacggggtgtaacagcgacgacaaatgcccctgcggtaacaagtctgaagaaaccaagaagtcatgctgctctgggaaatgagtatattagaaaggaggaatatataatgtttgcaaaaagatttaaaacatcactgctgccgctgtttgcaggctttctgctgctgtttcatctggttctggcaggcccggcagcagcatcagcactggaaatttcatcaacatgcgatgcacaaaaactgattctgacaggcagaccgtttctgcatcatcaaggcattattaatcaagtttcaacagttacaaaagttattcatcatgaactggaagttgcagcatcagcagatgatatttggacagtttattcatggccgggcctggcaaaacatctgccggatctgctgccgggcatttttgaaaaactggaaattattggcgatggcggcgttggcacaattctggatatgacatttgttccgggcgaatttccgcatgaatataaagaaaaatttattctggttgataatgaacatagactgaaaaaagttcaaatgattgaaggcggctatctggatctgggcgttacatattatatggatacaattcatgttgttccgacaggcaaagattcatgcgttattaaatcatcaacagaatatcatgttaaaccggaatttgttaaaattgttgaaccgctgattacaacaggcccgctggcagcaatggcagatgcaatttcaaaactggttctggaacataaatcaaaatcaaattcagatgaaattgaagcagcaattattacagttctgccggatacatcagcaggctattataatgaaatggttattggcgcagcagttacaatttcaggcacatatctggttagaagaaaaagagcatcaagaacataaggctggatcacctcctttctaaaaaaaaaccccgcccctgacagggcggggttttttttaattttgtcaaaataattttattgacaacgtcttattaacgttgatataatttaaattttatttgacaaaaatgggctcgtgttgtacaataaatgtgtatattagaaaggaggaatatataatggttgttattaatggcgttaaatatgcatgcgaaacatgcattagaggccatagagcagcacaatgcacacatacagatggcccgctgcaaatgattagaagaaaaggcagaccgtcaacaacatgcggccattgcaaagaactgagaagaacaaaaaattttaatccgtcaggcggctgcatgtgcgcatcagcaagaagaccggcagttggctcaaaagaagatgaaacaagatgcagatgcgatgaaggcgaaccgtgcaaatgccatacaaaaagaaaatcatcaagaaaatcaaaaggcggctcatgccatagaagagcaaatgatgaagcagcacatgttaatggcctgggcattgcagatctggatgttctgctgggcctgaatggcagatcatcagatgttgatatgacaacaacactgccgtcactgaaaccgccgctgcaaaatggcgaaattaaagcagattcaattgataatctggatctggcatcactggatccgctggaacaatcaccgtcaatttcaatggaaccggtttcaattaatgaaacaggctcagcatatacaacaacaaatacagcactgaatgatattgatattccgttttcaattaatgaactgaatgaactgtataaacaagtttcatcacataattcacattcacaataaggctggatcacctcctttctaaaaaaaaaccccgcccctgacagggcggggttttttttggcgtagaggatcgatcccggttcgacaccctgatatatatatatatatatatatatatatatatatatatatatatatacaggaacaaagtttttcctggacgccggtagaatatacacaaaaatggtttcgaccaggtttcgaccatgactgcatggtcgaaaccaaaaatggtttcgaccatgcagtcataagcaccatgggttgcaggactttacgccagtgtcctgcttaatttgtcatttaatgctatcagcagtcttcacccgtgttcggtccgacggtatgtgccccaccgtttaacttatggtgtcaatttgtcctccaaaccaacttggtagagtggtaaagtggtagaacggaccggatggatgggtggaagagccggttggacaacacaaatatacttgtatcacgaccagaaaagtgctatagacatcacatagacagtcatcggcttctgcctgcgttttttttctttcatcacacacatcatcaacctcatacattgcccatcaactaaaaaacaacgcagcagcggatagcccccaagcatttgcgaagaacgtaaaaacaaaacaaaaaagctcgacgaatggtttgcaaacatcacacgctgctcatttacgttagtattatcttttactgtatatatatatatatatatatatatacgataatgaaccattttgcagcagtttccctgaaaaacgaaaatgcgggaaattaatagtacggttgtgtagggtacgtctccgacgtttttgtttcttcaacattttaattgatttttggttgtcttcttactttcttttctctattatgtttcccgacctctctttcctttttgtaggctctctccgatgagatcctgcgtactcctctctggaaacggaaaccagaacaaggctaaataaaaaagaagtagtacgtagcgaaacaaggacgccggagctacgtcgcatatgctcacatacgtaatgtattttaaaaaacaagcaaaatccgagactgcaaaggctctgggtatcgtcggcgtttttccgccatttgctttacagaagttattgcgttccttcgtttttttgagcagtgtcgcgcatggccgcgccttatgtaccaaggcagacacgggttaacccagccttttggtgatggcctacgtcgttgaaacactgccctgcaagtatggggcgttctttttttccctccaagtcaattacggaagcaccccggatgcgttagtgtcgagttgcaccccgaaagagaaacgccacttgtgtgcgcgtgacttctgccgagcgcccaccatgagtcatctgcataccgcccatgtcctgacacgcgctttcggataagcggagtatcggatcgagctcctaaacagctgcggacctacctaaccactacatgcaatgtcgacgtagtgagctcaagtgctttttaacccaactgtgacctggtgggggagttcgccggacatgccacgctgccattgaatggctccgcacgaatagaccttctctcgagcaaacaaatacaccgaacgtggaaaaaaaaaaaaaaaaaaaaaggatggcaatggtcagccggtgtaacgaccaaaaactatccgccatgcattaaccaccatgcgagtggttccgacggttgctgtagagtttcaaagaaccttgttcgtcaatcacgctgttgcggctctgaattcagcaaattcacagtattccgcgcccattcagcccgataatgtcaggcggggtacagaattgatatttatagtaaactaaaccgtggacgaatcaggcgacgaatggttgtactggtgggtggtgtagggtggcagggaaagcattttgtacactagcctccctgcggcactccctgccttatttttgcctgcgccgagcacagacgagacgaaccaggtttagctggtttagtaccccaactgatggaaaatgctacgtgtgctggggctgaaacagagattttttgctagaatcttaattcgttcaaacacagaaaatgtttgggaaaaataagcttatcgcattgctcagttaattgatttctgcagaaagcgggccacgcagctgcgtttaggcgccaaaaattgaggcggccggcccactctccttcctgcaaaggtgttctgctgaagggaatcatgggaaagcgttgctcgtaatgaaataacccaacatggcgcctgcaaatgtgcgcagttctggactgcttgttttgagctcgtgaaaacaatatctggagcatcataccatcgttttaaagctataaacagcgtgtgattacgctaaacactgcccagcacgtggattgttgtcgcagacctcgagttgttgcaaagacaatggtactgcgaagcgtaacaggctcgtatacatgtaaggtgggatttggtaagcgtaactattggttttactagtatagctgcgtaggttcacgcgtgcaaggccgcttagtatgtgtttaccagccaatgtgctatgcaaaacaaaggaaaagtataaaaacggcctcgcagcccttggtggccaactgaaagtcaacaacaaacgtgatcacgtgacctcttagtaagcgctaatcaccgttaaattatatgaaattacacttcattgcgttttatctaaacatgaacgcagtttaaggctatgctaaacagttccgtacagaaatcaaacatttagagtagggagtgcaagcaactgggagaacagcggcggaatcgtatataaacggtttccgatcgtctccgccatgttttggtcgatgttatgcttcaacagtgattgaacgagcatcgccaaactgctttcctctctcccccaaatctttctcactgcatatttcttgaagaaagtacagttggacgttccgttccggaaataaccgacgaaagtatattagaaaggaggaatatataatgtttgcaaaaagatttaaaacatcactgctgccgctgtttgcaggctttctgctgctgtttcatctggttctggcaggcccggcagcagcatcagcactggaaatttcatcaacatgcgatgcaatgactgactttctcgaggttaatcggcaagagttagggagatggctgcaagaagaaccaggagcctttaactggagcatgtattcccagcatgcgtgtctgatagagggaaaaggcgaatgctggcaagataaggaacgacagttgcgagctcgtgttaaacgcgtactgccgattgacgtccaccaaccgcaaccgttaggtgcaggatctcctgcaccattaccggcggatgccttagtctcagccttttgcttggaagccgtttcaccggatctggcgtctttccagagagcgcttgatcacatcacgaccttgcttcgtcctggtggacatctccttctgattggcgcactggaagagagctggtatttggctggtgaagctcgccttacagtggtacccgtgtcggaggaagaagtgagggaagctctcgttagaagtgggtataaggttcgcgatttacggacgtacatcatgcctgcacatcttcagacaggcgtggatgacgtcaaaggcgtattctttgcatgggcgcagaaagtcggcctgctgccggatacatcagcaggctattataatgaaatggttattggcgcagcagttacaatttcaggcacatatctggttagaagaaaaagagcatcaagaacataaggctggatcacctcctttctaaaaaaaaaccccgcccctgacagggcggggttttttttctactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtcttgacaaattgcagtaggcatgacaaaatggactcagtatattagaaaggaggaatatataatggtgtctaaaggcgaagaggttatcaaggagtttatgcgctttaaggtacggatggaaggatcagtaaatggccacgaattcgaaattgagggtgaaggggaaggaaggccgtatgaagggacacaaactgcgaaactgaaagtcacaaaaggaggtcctttaccctttgcctgggatatcttaagcccacaatgcatgtatggcagcaaaggctacgtgaaacatccagctgacattccggattatctcaaactgagctttccggaaggcttcaaatgggaaagagtgatgaactttgaggatggaggagtagtgactgtcacccaggactctagtttgcaggatggagaattcatctacaaggtcaagcttcgtggaaccaactttccgtcagatggccctgttatgcaaaagaaaacgatggggtgggaagcttcgtcagaacgtatgtatcctgaggatggtgcacttaaaggcgagatgaaaatgcgcttgaaactgaaagatggaggccattatgacgcagaagtcaaaacgacgtatatggcgaagaaaccggttcaactgcctggtgcctacaaaacagacattaagcttgacattacatcccacaatgaagattacacgatagttgagcagtatgaaagagcagaagggcgacattccacaggtgcgtaaggctggatcacctcctttctaaaaaaaaaccccgcccctgacagggcggggttttttttcccgaacctgttggaacggctccttcagcatgatataagtaaattgtaagtatattagaaaggaggaatatataatgaaaaaaattgcaattgcagcaattacagcaacatcaattctggcactgtcagcatgctcaggcggcgattcagaagttgttgcagaaacaaaagcaggcaatattacaaaagaagatctgtatcaaacactgaaagataatgcaggcgcagatgcactgaatatgctggttcaaaaaaaagttctggatgataaatatgatgtttcagataaagaaattgataaaaaactgaatgaatataaaaaatcaatgggcgatcaactggatcaactgattaaacaaaaaggcgaagattttgttaaagaacaaattaaatatgaactgctgatgaaaaaagcagcaaaagataatattaaagttacagatgatgatgttaaagaatattatgatggcctgaaaggcaaaattcatctgtcacatattctggttaaagaaaaaaaaacagcagaagaagttgaaaaaaaactgaaaaaaggcgaaaaatttgaagatctggcaaaagaatattcaacagatggcacagcagaaaaaggcggcgatctgggctgggttggcaaagatgataatatggataaagattttgttaaagcagcatttgcactgaaaacaggcgaaatttcaggcccggttaaatcacaatttggctatcatattattaaaaaagatgaagaaagaggcaaatatgaagatatgaaaaaagaactgaaaaaagaagttgaagaacaaaaactgaatgatcaaacagaactgcaatcagttattgataaactggttaaagatgcagatctgaaagttaaagataaagaactgaaaaaacaagttgatcaaagacaagcacaaacatcatcatcatcataaggctggatcacctcctttctaaaaaaaaaccccgcccctgacagggcggggtttttttt

SEQ ID NO: 2 - protein, 67 aa, Artificial Sequence
  Synthetic Construct
QLDGYIIHFFFTTGTELARAGPGAFFKYPREIELIVKTNIATDGGDRHPGGAQKQLRLADTLVLAPA

SEQ ID NO: 3 - protein, 4 aa, Artificial Sequence
  Synthetic Construct
DANP

SEQ ID NO: 4 - protein, 6 aa, Artificial Sequence
  Synthetic Construct
LLAEKM

SEQ ID NO: 5 - protein, 51 aa, Artificial Sequence
  Synthetic Construct
QTRRRQANMLCDAGDIKIAVCQVIADVLTSLAYPIIHRWMERLVNRFHAPQ

SEQ ID NO: 6 - protein, 47 aa, Artificial Sequence
  Synthetic Construct
QLLKQIYRQQLRIAPFPLPGVNDLPKQVAEMRLVRFIRAKEPRIGKY

SEQ ID NO: 7 - protein, 88 aa, Artificial Sequence
  Synthetic Construct
RPVKPFMPVGARTKVNPLVIPFASLRMTTVVMNLSWREQQNITRSANKFSSLIFHHPLTANGEIENITFHSQRSVDKKIEITVGLNRR

SEQ ID NO: 8 - protein, 22 aa, Artificial Sequence
  Synthetic Construct
TRHQMGIKRVSRQQGIILRFSH

(19) United States
(12) Patent Application Publication
(10) Pub. No.: US 2024/0246965 A1
(43) Pub. Date: Jul. 25, 2024

(54) NOVEL HETEROCYCLIC COMPOUNDS AND THEIR USE

(71) Applicant: Equinorm Ltd, Espoo (FI)

(72) Inventor: Ville Takio, Espoo (FI)

(73) Assignee: Equinorm Ltd, (omitted)

(21) Appl. No.: 18/553,471

(22) PCT Filed: Mar. 30, 2022
(86) PCT No.: PCT/FI2022/050205

§ 371 Date: Sep. 29, 2023

(30) Foreign Application Priority Data
Mar. 31, 2021 (FI) 20215387

(51) Int. Cl. C07D 471/04; A61K 31/437; A61K 31/4375; A61K 31/4745; C07D 455/00

(52) U.S. Cl. CPC C07D 471/04; A61K 31/437; A61K 31/4375; A61K 31/4745; C07D 455/00

(57) ABSTRACT

The present invention relates compounds of general formula (I)

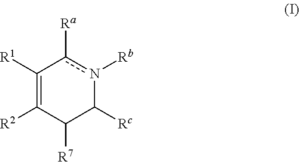

and stereoisomers and pharmaceutically acceptable salts thereof; wherein R¹, R², R⁷, R^{a}, R^{b}, R^{c}, and the dotted line is as defined in the claims. The invention also relates to pharmaceutical compositions comprising a compound of formula (I) and to said compounds for use as a medicament and particularly in the treatment or prevention of drug addiction and CNS related diseases and conditions. Further, the invention relates to methods for the preparation of a compound of formula (I), or pharmaceutically acceptable salt or a stereoisomer thereof.

### CROSS-REFERENCE TO RELATED APPLICATIONS

This application is a national phase entry of International Patent Application No. PCT/FI2022/050205 (filed 30 Mar. 2022), which claims priority to Finnish Patent Application No. 20215387 (filed 31 Mar. 2021), the entire disclosures of which are incorporated herein by reference.

### BACKGROUND

### Technical Field

The present invention relates to novel compounds derived from substituted 1,2,3,6-tetrahydropyridines and 2,3-dihydropyridines and pharmaceutical compositions comprising said compounds. More particularly, the present invention relates to novel compounds derived from 1,2- or 2,3-disubstituted 1,2,3,4-tetrahydroisoquinolines and -2,3,4,9-tetrahydro-1H-pyrido[3,4-b]indoles, and 1-methyl-4,9-dihydro-3H-, and 1-methyl-2,3,4,9-tetrahydro-1H-pyrido[3,4-b]indoles. The invention also relates to said compounds for use in the treatment or prevention of drug addiction and CNS related diseases, in addition to methods for the preparation of said derivatives.

### Discussion of Art

Among endogenous tetrahydropyridine derivatives, tetrahydroharman (1-methyl-1,2,3,4-tetrahydro-ß-carboline) is an indole alkaloid that lowers blood pressure. Reserpine, another indole alkaloid with a tetrahydropyridine structure, is used as a drug for treating hypertension and has also shown to exhibit effects in relieving psychotic symptoms. Furthermore, known tetrahydropyridine derivatives with a fused indole ring are tryptoline (tetrahydro-β-carboline, 1,2,3,4-tetrahydro-9H-pyrido[3,4-b]indole), pinoline (5-methoxytryptoline), tetrahydroharmine ((1R)-7-methoxy-1-methyl-2,3,4,9-tetrahydro-1H-pyrido[3,4-b]indole), harmaline (7-methoxy-1-methyl-4,9-dihydro-3H-pyrido[3,4-b]indole), and tetrahydroharmine ((1R)-7-methoxy-1-methyl-2,3,4,9-tetrahydro-1H-pyrido[3,4-b]indole) that show various effects including psychoactivity, monoamine oxidase inhibition activity, serotonin reuptake inhibition activity, stimulating the central nervous system and promotion of neurogenesis. Among synthetic tetrahydropyridine derivatives, gevotroline (8-fluoro-2-(3-(pyridin-3-yl)propyl)-2,3,4,5-tetrahydro-1H-pyrido[4,3-b]indole) is an atypical antipsychotic that was developed for the treatment of schizophrenia, however, the compound never research the market. Another tetrahydropyridine derivative, latrepirdine (dimebolin, 2,8-dimethyl-5-(2-(6-methylpyridin-3-yl)ethyl)-2,3,4,5-tetrahydro-1H-pyrido[4,3-b]indole), is an antihistamine drug that failed in phase III clinical trials for both the treatment of Alzheimer's and Huntington's disease.

The related endogenous isoquinolines show a various of effects including sedative, psychotropic and analgesic properties, morphine and codeine being well known examples of isoquinoline alkaloids. Endogenous isoquinolines are formed by condensation of biogenic amines—such as phenethylamine—and simple aldehydes, such as formaldehyde or acetaldehyde. They are known to modulate neurotransmission, central metabolism and motor activity. An endogenous tetrahydroisoquinoline (TIQ) derivative salsolinol (SAL, 1-methyl-1,2,3,4-tetrahydroisoquinoline-6,7-diol) is considered to be a causative factor of Parkinson's disease while (PD), (R)-1MeTIQ (1-methyl-1,2,3,4-tetrahydroisoquinoline) was shown to possess an antiparkinsonian activity. Until recently 1-MeTIQ was the only known neuroprotective/PD preventing TIQ derivative. In 2006 Katsuhiro OKUDA et al. (Biological and Pharmaceutical Bulletin 29 (2006) pp. 1401-1403) discovered that 5-/6-/7-monohydroxylated 1MeTIQ derivatives are neuroprotective and PD preventing indeed, even more so than the parent compound.

It has been demonstrated that concentrations of many endogenous TIQ derivatives are significantly elevated in the urine and cerebrospinal fluid of PD/ADHD (attention deficit hyperactivity disorder) patients compared to controls, the content of 1MeTIQ however is significantly decreased in PD patients' cerebrospinal fluid and brain.

SAL is formed enzymatically as well as non-enzymatically as a condensation product of acetaldehyde—the primary metabolite of ethanol—with dopamine in the brain of mammals. SAL affects the uptake of catecholamines into nerve terminals, the release of stored catecholamines and the activity of monoamine oxidase (MAO), catechol-O-methyl transferase (COMT) and tyrosine hydroxylase. Ethanol induced elevation of salsolinol levels is known to participate in the development of ethanol addiction/alcoholism.

SAL has been postulated to mediate some of the addictive properties of alcohol. A number of studies have shown that primates self-administer SAL even in nanomolar concentrations when intracranially injected to certain brain region. Also acetaldehyde is self-administered when injected intracranially although much higher concentrations are needed. Studies have confirmed that SAL is released during suckling on lactating sheep. It is clear that SAL mediates reinforcing effects on a number of primate species. Studies have shown that controlled amounts of ethanol intake have only miniscule effect on brain SAL levels. Still it is clear that ethanol intake elevates dopamine and acetaldehyde concentrations inside brain and so the concentrations of starting materials for Pictet-Spengler reaction forming SAL are elevated. In this light it seems certain that alcoholics with higher ethanol intake and generally higher aldehyde dehydrogenase (ALDH) activity (see Alcohol Clin. Exp. Res. 2009 November; 33(11):1935-44) try to compensate lower acetaldehyde and SAL concentrations by increased drinking.

Most TIQs penetrate to the brain in pharmacologically relevant amounts and induce a variety of effects. Most of the TIQ and 1MeTIQ exit the brain (90.4% and 95.3%) and is excreted in urine (76% and 72%) unchanged. The hydroxylated (C4 of the isoquinoline backbone) derivatives of TIQ and 1MeTIQ were the most abundant metabolites in the urine (2.7% and 8.7%).

In European Journal of Medicinal Chemistry 41 (2006) pp. 241-252 6-fluoro-1-methyl-1,2,3,4-tetrahydroisoquinoline has been reported as a possible agent against Parkinson's disease.

EP 2 090 576 A1 discloses certain 5,8- and 6,7-difluoro substituted isoquinolines as intermediates in the preparation of pyrazolo[1,5-a]pyridines for use as metabotropic glutamate receptor modulators.

WO 2002028865 A2 discloses certain specifically substituted 2,3,4,9-tetrahydro-1H-pyrido[3,4-b]indoles as selective inhibitors of cyclic guanosine 3′,5′-monophosphate specific phosphodiesterase.

EP 3 459 950 A1 discloses certain β-carboline, dihydro-β-carboline and tetrahydro-β-carboline alkaloid compounds with anti-plant virus activity and are disclosed to also have fungicidal and insecticidal activities.

WO 2012/020170 discloses 6,7-disubstituted-1-methyl-1,2,3,4-tetrahydro- and -3,4-dihydroisoquinolines for use in the treatment of drug addiction and CNS related diseases.

It has now surprisingly been found that specifically substituted 1,2,3,6-tetrahydropyridines and 2,3-dihydropyridines blocks the adverse metabolic formation of dihydroxy compounds and thereby also improves the desired activity of the isoquinoline derivative in question.

### BRIEF DESCRIPTION

An object of the present invention is to provide compounds useful in treating disorders and diseases associated with drug addiction and CNS related diseases.

One of the problems associated with the known compounds is the disposition, in particular the metabolic stability, of the compounds. It is therefore yet a further object of the present invention to provide compounds with improved metabolic stability.

The invention is based on the realization that the compounds of the invention bind to and/or affect the activity of proteins that are associated with drug addiction and CNS related diseases.

The objects of the invention are achieved by compounds and said compounds for use as a medicament that are characterized by what is stated in the independent claims. The preferred embodiments of the invention are disclosed in the dependent claims.

The present invention provides novel compounds of formula (I)

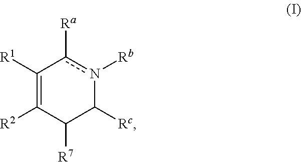

wherein the dotted line represents an optional bond;
R¹ and R², together with the carbon atoms they are attached to, form a group selected from a 1H-indole group and a benzene group, and said 1H-indole group and benzene group being optionally substituted with one to four substituent(s) each independently selected from the group consisting of R3, R4, R5, and R6, wherein each R3, R4, R5, and R6 is independently selected from the group consisting of halogen, OH, C1-4-alkyl, C1-3-(per)haloalkyl, C1-3-alkoxy, C1-3-(per)haloalkoxy;
Ra and Rb, together with the carbon atom and nitrogen atom they are attached to, form a group selected from a 5- and 6-membered cyclic amide, and
Rc is H; or
Ra is Me, and
Rb and Rc, together with the nitrogen atom and carbon atom they are attached to, form a group selected from a 5- and 6-membered cyclic amide; or
Ra is Me,
Rb is H, or Rb is absent when the dotted line represents a bond, and
Rc is H, provided that R1 and R2, together with the carbon atoms they are attached to, form said optionally substituted 1H-indole group;
R7 is selected from the group consisting of halogen, OH, oxo, SH, NOR8, C1-3-(per)haloalkyl, C1-3-alkoxy, C1-3-(per)haloalkoxy, CN, C(O)N(R8)2, and N(R8)2, or
R7 may also be C1-4-alkyl with the provisio that said 1H-indole group or benzene group is substituted with one to four substituent(s) each independently selected from the group consisting of halogen, OH, C1-4-alkyl, C1-3-(per)haloalkyl, C1-3-alkoxy, C1-3-(per)haloalkoxy, or
R7 may also be H provided that
when Ra and Rb, together with the carbon atom and nitrogen atom they are attached to, form a group selected from a 5- and 6-membered cyclic amide, Rc is H, then R4 and R5 is each independently selected from the group consisting of halogen, C1-4-alkyl, C1-3-(per)haloalkyl, and C1-3-(per)haloalkoxy, or
when Ra is Me, Rb and Rc, together with the carbon atom and nitrogen atom they are attached to, form a 6-membered cyclic amide, then R1 and R2, together with the carbon atoms they are attached to, form said optionally substituted 1H-indole group, or
when Ra is Me, Rb and Rc, together with the carbon atom and nitrogen atom they are attached to, form a 5-membered cyclic amide, and R1 and R2, together with the carbon atoms they are attached to, form a 1H-indole group or a benzene group, then said 1H-indole group or benzene group is substituted with one to four substituent(s) each independently selected from the group consisting of halogen, OH, C1-4-alkyl, C1-3-(per)haloalkyl, C1-3-alkoxy, and C1-3-(per)haloalkoxy, or
when Ra is Me, Rc is H, R1 and R2, together with the carbon atoms they are attached to, form a substituted 1H-indole group, then R4 and R5 of said substituted 1H-indole group is each independently selected from the group consisting of halogen, C1-4-alkyl, C1-3-(per)haloalkyl, and C1-3-(per)haloalkoxy;
each R8 is independently selected from the group consisting of H, C1-4-alkyl, C1-4-alkenyl, C1-4-alkynyl, and C1-3-(per)haloalkyl, or when part of any N(R8)2 both R8 together with the nitrogen they are attached to may form a 3- to 6-membered aliphatic or aromatic heterocyclic ring comprising 1 to 3 heteroatoms each independently selected from N, O, and S;
or a stereoisomer or a pharmaceutically acceptable salt thereof.

The invention also relates to pharmaceutical compositions comprising an effective amount of one or more compound(s) of formula (I), or a stereoisomer or a pharmaceutically acceptable salt thereof, together with one or more pharmaceutically acceptable excipient(s).

Further the invention relates to a compound of formula (I), or a stereoisomer or pharmaceutically acceptable salt thereof, for use as a medicament.

Further, invention relates to a compound of formula (I), or a stereoisomer or pharmaceutically acceptable salt thereof, for use in treatment or prevention of CNS related diseases or conditions.

The invention also relates to a compounds of formula (I), or a stereoisomer or pharmaceutically acceptable salt thereof, for use in the treatment or prevention of a disease selected from the group consisting of Alzheimer's disease, Parkinson's disease, depression, anxiety, hyperactivity, narcolepsy, drug addiction, alcoholism, anorexia, bulimia, and mitochondrial disease.

Finally, the invention provides a method for the preparation of compounds of formula (I).

### DETAILED DESCRIPTION

Compounds of the invention are derivatives of 1,2,3,6-tetrahydropyridine and 2,3-dihydropyridine that are fused at C4 and C5 to either an optionally substituted 1H-indole ring or an optionally substituted benzene ring that, together with the specific substitution pattern of the 1,2,3,6-tetrahydropyridine and 2,3-dihydropyridine rings, provide the inventive properties of the compounds of the present invention. Also, the substitution of said 1H-indole and benzene ring further enhance the metabolic and/or inhibitory properties of the compounds of the present invention.

The term “halogen” as used herein and hereafter by itself or as part of other groups refers to the Group VIIa elements and includes F, Cl, Br and I groups, preferably F.

The term “alkyl” as used herein and hereafter is an aliphatic linear, branched or cyclic, especially linear or branched, hydrocarbon group having the indicated number of carbon atoms, for example C₁₋₆-alkyl has 1 to 6 carbon atoms in the alkyl moiety and thus, for example, C₁₋₄-alkyl includes methyl, ethyl, n-propyl, isopropyl, n-butyl, sec-butyl, isobutyl, tert-butyl and C₁₋₆-alkyl additionally includes branched and straight chain pentyl and hexyl. Preferably the alkyl is methyl or ethyl.

The term “C₁₋₄-alkenyl” as used herein and hereafter is an unsaturated linear or branched hydrocarbon group having at least one olefinic double bond between any two carbon atoms and having suitably 1 to 4, preferably 1 to 3, carbon atoms in the alkenyl moiety, such as ethenyl, 1-propenyl, 2-propenyl, 3-propenyl, and butenyl. Examples of preferred alkenyls groups include, but are not limited to, linear alkenyl groups having a terminal double bond such as vinyl and allyl groups.

The term “C₁₋₄-alkynyl” as used herein and hereafter is an unsaturated linear or branched hydrocarbon group having at least one olefinic triple bond between any two carbon atoms and having suitably 1 to 4, preferably 1 to 2, carbon atoms in the alkenyl moiety, such as ethynyl, propynyl, and butynyl.

The term “C₁₋₃-(per)haloalkyl” as used herein and hereafter refers to any of the above alkyl groups where one or more hydrogen atoms are replaced by halogen(s): in particular I, Br, F or Cl. Examples of haloalkyl groups include without limitation chloromethyl, fluoromethyl and —CH₂CF₃. The term “perhaloalkyl” is understood to refer to an alkyl group, in which all the hydrogen atoms are replaced by halogen atoms. Preferred examples include trifluoromethyl (—CF₃) and trichloromethyl (—CCl₃).

The term “C₁₋₃-alkoxy” as used herein and hereafter refers to a —O—(C₁₋₃-alkyl) group where the “C₁₋₃-alkyl” has the above-defined meaning. Examples of preferred alkoxy groups include, but are not limited to, methoxy, ethoxy, and iso-propyloxy.

The term “C₁₋₃-(per)haloalkoxy” as used herein and hereafter refers to a —O—(C₁₋₃-(per)haloalkyl) group where the C₁₋₃-(per)haloalkyl has the above-defined meaning. Examples of preferred alkoxy groups include, but are not limited to, trifluoromethoxy, 2,2,2-trichloromethoxy, and 1,1,1,3,3,3-hexafluoro-isopropoxy.

The term “5- and 6-membered cyclic amide” as used herein and hereafter refers to a 5- and 6-membered lactam that may or may not comprise other heteroatoms, such as N, O, and S, and substituents, such as methyl, hydroxy, amine, thiol and methoxy. Examples of preferred 5- and 6-membered cyclic amide groups include, but are not limited to, 2-pyrrolidone, 2-piperidinone, imidazolidin-2-one, oxazolidin-2-one, oxazolidin-4-one, imidazolidin-4-one, tetrahydropyrimidin-2(1H)-one, 1,3-oxazinan-2-one, piperazin-2-one, thiomorpholin-3-one, 4-methylpiperidin-2-one, and 5-hydroxypiperidin-2-one.

The term “oxo” as used herein and hereafter refers to a functional group “═O”, which, together with the carbon atom to which the oxygen of “oxo” is attached to and a carbon-oxygen double bond between said carbon atom and said oxygen atom, forms a carbonyl group of a compound disclosed herein. Therefore, when R⁷ is “oxo”, it is to be understood that the carbon atom to which the oxygen atom of “oxo” is attached to, does not contain one or more hydrogen atoms.

The term “NOR⁸” as used herein and hereafter refers to a functional group, which, together with the carbon atom to which the nitrogen of NOR⁸ is attached to and a carbon-nitrogen double bond between said carbon atom and said nitrogen atom, forms an oxime functional group of a ketoxime compound disclosed herein and said oxime functional group may or may not be substituted at the oxygen atom of the oxime functional group, wherein R⁸ is as defined herein and hereafter. Therefore, when R⁷ is NOR⁸, it is to be understood that the carbon atom to which the nitrogen atom of NOR⁸ is attached to, does not contain one or more hydrogen atoms. Said NOR⁸ may be synthesized e.g. by condensation of a compound comprising a carbonyl group (R⁷ is oxo) with e.g. hydroxylamine, forming a compound, wherein R⁷ is NOH. Examples of NOR⁸ groups include, but are not limited to, hydroxyimino, methoxyimino, (trifluoromethoxy)imino, and (2,2,2,-trifluoroethoxy)imino, and (E)- and (Z)-isomers thereof.

The term “3- to 6-membered aliphatic or aromatic heterocyclic ring comprising 1 to 3 heteroatoms each independently selected from N, O, and S” as used herein and hereafter refers to a monocyclic ring which is saturated, partially unsaturated, unsaturated or aromatic with 3 to 6 ring atoms that may or may not comprise one or more double bond between the ring atoms and said monocyclic ring comprises 1 to 3 heteroatom(s) each independently selected from the group consisting of N, S, and O, while the remaining ring atoms are carbon atoms. It may be substituted with one to four substituent(s) at any suitable ring atom, including N. Preferred substituents groups include, but are not limited to halogen, in particular fluoro, CN, methoxy, hydroxy, amino, and methyl. Examples of heterocyclic rings include, but are not limited to, aziridinyl, azetidinyl, 1,3-diazetidinyl, pyrazolidinyl, imidazolidinyl, imidazolyl, piperidinyl, dihydrothiazolyl, piperazinyl, pyrrolidinyl, thiomorpholinyl, dioxide of thiomorpholinyl, and methoxymethylpyrrolidinyl.

The term “optionally substituted” as used herein and hereafter denotes that the group it refers to is either unsubstituted or substituted independently with one or more, preferably 1, 2, 3 or 4, substituent(s) attached at any available atom to produce a stable compound. E.g. phenyl may be substituted once with a denoted substituent attached to o-, m- or p-position of the phenyl ring. In general, “substituted” refers to a substituent group as defined herein in which one or more bonds to a hydrogen atom contained in the group if refers to are replaced by a bond to a non-hydrogen atom of the substituent unless otherwise denoted. Preferred substituents groups are each independently selected from the group consisting of, but not limited to, halogen such as F, Cl, Br and I, in particular F and Cl; CO₂H and esters thereof; C₁₋₄-alkyl, in particular methyl; OH, C₁₋₃-alkoxy, in particular OMe, OEt and OCHCH═CH₂; NO₂, N₃, NOH, and ethers thereof, in particular NOMe; CN, NH₂, and amides thereof, in particular NHC(O)Me; NH(C₁₋₆-alkyl), N(C₁₋₆-alkyl)₂, N⁺(C₁₋₆-alkyl)₃, in particular NHMe, N(Me)₂, N⁺(Me)₃, and salts thereof; C(O)N(C₁₋₆-alkyl), in particular C(O)NHMe; NHC(O)—C₁₋₆-alkyl, in particular NHC(O)Me; SH, and thioethers thereof; C₁₋₄-alkenyl, C₁₋₄-alkynyl, C₁₋₃-(per)haloalkyl, in particular CF₃ and CH₂CF₃; C₁₋₃-(per)haloalkoxy, in particular OCF₃ and OCH₂CF₃; SC(O)—C₁₋₆-alkyl, OC(O)—C₁₋₆-alkyl, NHC(O)NH—C₁₋₆-alkyl, NHC(O)O—C₁₋₆-alkyl. Preferably said substituent group is optionally substituted with OH, NH₂, CO₂H and halogen.

“Optional” or “optionally” denotes that the subsequently described event or circumstance may but need not occur, and that the description includes instances where the event or circumstance occurs and instances in which it does not. “Comprises” or “comprising” denotes that the subsequently described set may but need not include other elements.

The term “stereoisomer” as used herein and hereafter refers to stereoisomers of compounds. Examples of stereoisomers include, but are not limited to, enantiomers, diastereomers, cis-trans-isomers, and E-Z-isomers.

The term “pharmaceutically acceptable salt” as used herein and hereafter refers to salts which are known to be non-toxic and are commonly used in the pharmaceutical literature. Typically, these are acid addition salts or base addition salts of the referred compounds of the invention.

The expression “acid addition salt” includes any non-toxic organic and inorganic acid addition salts that that the compounds of formula (I)—(V), (Ia), (Ib), (Ic), (Id), (Ie), (If), (Ig), and (I′) can form. Illustrative inorganic acids, which form suitable acid addition salts, include, but are not limited to, hydrogen chloride, hydrogen bromide, sulphuric and phosphoric acids. Illustrative organic acids, which form suitable acid addition salts, include, but are not limited to, acetic acid, lactic acid, malonic acid, succinic acid, glutaric acid, fumaric acid, malic acid, tartaric acid, citric acid, ascorbic acid, maleic acid, benzoic acid, phenylacetic acid, cinnamic acid, methane sulfonic acid, salicylic acid, and the like. The term “acid addition salt” as used herein also comprises solvates which the compounds and salts thereof are able to form, such as, for example, hydrates, alcoholates, and the like. These salts also include salts useful for the chiral resolution of racemates.

The expression “base addition salt” includes any non-toxic base addition salts that the compounds of formula (I)—(V), (Ia), (Ib), (Ic), (Id), (Ie), (If), (Ig), and (I′) can form. Suitable base addition salts include, but are not limited to, those derived from inorganic bases such as aluminum, ammonium, calcium, copper, iron, lithium, magnesium, manganese, potassium, sodium, and zinc salts, in particular sodium and ammonium salts. Further examples of organic base addition salts include salts of trialkylamines, such as triethyl amine and trimethyl amine, other salts of organic amines such as methylamine, dimethylamine, trimethylamine, ethylamine, triethylamine, morpholine, and the like, and choline salts.

Pharmaceutical compositions of the present invention may be administered in an effective amount within the dosage range of about 0.1 μg/kg to about 300 mg/kg, preferably between 1.0 μg/kg to 10 mg/kg body weight. Compounds of the present invention may be administered in a single daily dose, or the total daily dosage may be administered in divided doses of two, three or four times daily.

The term “effective amount” refers to an amount of a composition or a pharmaceutical composition that confers a therapeutic effect on the treated subject. The therapeutic effect may be objective (i.e. measurable by some test or marker) or subjective (i.e. subject gives an indication of or feels an effect). Such treatment need not necessarily completely ameliorate the condition or disease. Further, such treatment or prevention can be used in conjunction with other traditional treatments for reducing the condition or disease known to those skilled in the art. The effective amount will typically be determined by a physician, and depend on the condition or disease to be treated, the chosen route of administration, the actual compound administered, the age, gender, weight, and response of the individual patient, the severity of the patient's symptoms, and like.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients.

Suitable pharmaceutically acceptable excipients include, but are not limited to, the following types of excipients: diluents (for example starches, mannitol), fillers (for example lactose, microcrystalline cellulose or calcium hydrogen phosphate), binders (for example pre-gelatised corn starch, polyvinylpyrrolidone or methylcellulose), additives (for example magnesium stearate, talc, silica), disintegrants (for example potato starch), lubricants (for example sodium lauryl sulphate), glidants (for example fumed silica, talc, magnesium carbonate), granulating agents (for example water, ethanol), coating agents (for example hydroxypropyl methylcellulose, gelatin, waxes, shellac, plastics, plant fibers), wetting agents (for example sorbitan monopalmitate, poloxamer 407), solvents (for example water), co-solvents (for example ethanol, propylene glycol), suspending agents (for example sorbitol, cellulose derivatives, edible hydrogenated fats), emulsifiers (for example lecithin or acacia), sweeteners (for example sucrose), flavoring agents (for example cherry, lime), flavor masking agents (for example vanilla, citrus), coloring agents (for example titanium oxide), anti-caking agents (for example silicon dioxide), humectants (for example glycerine, sorbitol), chelating agents (for example EDTA salts, histidine, aspartic acid), plasticizers (for example tributyl citrate, diethyl phthalate), viscosity increasing agents (for example methylcellulose), antioxidants (for example (ascorbic acid, cysteine), preservatives (for example methyl or propyl p-hydroxybenzoates, sorbic acid or ascorbic acid), stabilizers (for example polysorbate 20 & 80, poloxamer 407), surfactants (for example polyethylene glycol, polysorbate 80), and buffering agents (for example sodium and potassium phosphates, citrate, acetate, carbonate or glycine buffers de-pending on the targeted pH-range). Excipients and/or auxiliaries may facilitate processing of the active agent(s) into preparations that can be used pharmaceutically. The skilled artisan will appreciate that certain pharmaceutically acceptable excipients may serve more than one function and may serve alternative functions depending on how much of the excipient is present in the pharmaceutical composition and what other ingredients are present in the pharmaceutical composition.

Pharmaceutical compositions of the invention are most preferably used alone or in combination i.e. administered simultaneously, separately or sequentially with other active ingredients, e.g. pharmaceutically active compounds or biologic products. The amounts of the pharmaceutical composition(s) of the invention, particularly a pharmaceutical composition comprising a compound of formula (I), or pharmaceutically acceptable salts thereof, and the other active ingredient(s) and the relative timings of administration will be selected in order to achieve the desired combined therapeutic effect. Pharmaceutical compositions of the invention may be administered by various routes, for example, parenteral, subcutaneous, intravenous, intraarticular, intrathecal, intramuscular, intraperitoneal, topical, and by intradermal injections, and via transdermal, rectal, buccal, oromucosal, nasal, ocular routes and via inhalation and via implant.

Pharmaceutical compositions may be formulated into suitable pharmaceutical formulations; suitable administration forms include, for example, solutions, dispersions, suspensions, powders, capsules, tablets, pills, controlled release capsules, controlled release tablets and controlled release pills. In addition, or alternatively, to pharmaceutically acceptable excipient(s) and/or other active ingredients(s), the pharmaceutical formulations of the pharmaceutical compositions may contain one or more suitable pharmaceutically acceptable carrier(s).

The term “pharmaceutically acceptable carrier(s)” as used herein and hereafter refers to substrates comprised in pharmaceutical compositions for drug delivery, which serves to improve the selectivity, effectiveness, and/or safety of drug administration. Examples of pharmaceutically acceptable carriers include, but are not limited to, pharmaceutically acceptable excipients, liposomes, (polymeric) micelles, microspheres, nanoparticles, and protein-drug conjugates.

The pharmaceutical compositions of the invention are prepared using techniques and methods known to those skilled in the art. Pharmaceutical compositions of the invention include, but are not limited to, for parenteral and topical administration that include, but are not limited to, sterile aqueous or non-aqueous solvents, suspensions and emulsions. Examples of non-aqueous solvents are propylene glycol, polyethylene glycol, vegetable oil, fish oil, and injectable organic esters. Aqueous carriers include, but are not limited to, water, water-alcohol solutions, including saline and buffered medial parenteral vehicles including sodium chloride solution, Ringer's dextrose solution, dextrose plus sodium chloride solution, Ringer's solution containing lactose, or fixed oils. Intravenous vehicles include, but are not limited to, fluid and nutrient replenishers, electrolyte replenishers, such as those based on Ringer's dextrose and the like. Aqueous pharmaceutical compositions according to the invention may comprise suitable buffer agents, such as sodium and potassium phosphates, citrate, acetate, carbonate or glycine buffers depending on the targeted pH-range. The use of sodium chloride as a tonicity adjuster is also useful. Pharmaceutical compositions may include other excipients, such as stabilizing agents or preservatives. Useful stabilizing excipients include surfactants (polysorbate 20 & 80, poloxamer 407), polymers (polyethylene glycols, povidones), carbohydrates (sucrose, mannitol, glucose, lactose), alcohols (sorbitol, glycerol propylene glycol, ethylene glycol), suitable proteins (albumin), suitable amino acids (glycine, glutamic acid), fatty acids (ethanolamine), antioxidants (ascorbic acid, cysteine etc.), chelating agents (EDTA salts, histidine, aspartic acid) or metal ions (Ca, Ni, Mg, Mn). Among useful preservative agents are benzyl alcohol, chlorobutanol, benzalkonium chloride and possibly parabens. The pharmaceutical composition according to the present invention may be provided in concentrated form or in form of a powder to be reconstituted on demand. In such cases formulations of powder for solution for injection/infusion excipients mentioned above may be used. In case of lyophilizing, certain cryoprotectants are preferred, including polymers (povidones, polyethylene glycol, dextran), sugars (sucrose, glucose, lactose), amino acids (glycine, arginine, glutamic acid) and albumin. If solution for reconstitution is added to the packaging, it may consist e.g., of pure water for injection or sodium chloride solution or dextrose or glucose solutions.

The term “treatment or prevention” as used herein and hereafter includes prophylaxis, or prevention of, as well as lowering the individual's risk of falling ill with the named disorder or condition, or alleviation, amelioration, elimination, or cure of the said disorder once it has been established.

The terms “administering” or “administered” to a subject or patient includes dispensing, delivering or applying the composition or pharmaceutical composition to the subject by any suitable route for delivery of the composition or pharmaceutical composition to a site in the body where desired.

Compounds of formula (I) of the present invention may be useful in therapy, especially in the treatment or prevention of CNS related diseases and conditions in animals, in particular mammals, and humans. In particular, compounds of formula (I) possess pharmacological properties for the treatment and/or prophylaxis of CNS related diseases or conditions that include, but are not limited to, Alzheimer's disease, Parkinson's disease, depression, anxiety, hyperactivity, narcolepsy, drug addiction, alcoholism, anorexia, bulimia, mitochondrial disease, obesity, and multiple sclerosis.

The compounds of formula (I) of the present invention bind to one or more protein involved in a CNS related disease or condition, wherein examples of the proteins include, but are not limited to, trace amine receptor, dopamine and serotonine receptors, their respective transporters and acyl and methyl transferases, norepinephrine transporter, monoamino-oxidases, catecholine-O-methyltransferase, adrenergic receptors, tyrosine hydroxylase, histamine receptors, orexin receptors, NMDA-receptors, sigma-1 receptor, muscarinic and nicotinic acetylcholine receptors, opioid receptors, neuropeptide receptors, melanocortin receptors (excluding MC3R), neurokinin receptors and Corticotropin-releasing factor receptor 1 to name a few.

The term “protected with a protecting group” as used herein and hereafter refers to an atom or a functional group that is covalently attached to, or has been modified by, a protecting group. Said protecting group enables chemoselectivity in a reaction, therefore, the protecting group protects an atom or a functional group from reacting in a reaction. It is to be understood that the protecting group protects the atom or functional group fully or partly, i.e. the atom or the functional group protected with a protecting group may react partly in a reaction. Skilled artisans possess the knowledge and skill in the art to enable them to select suitable protecting groups for the atoms or functional groups to be protected. In addition, there are a number of resources that are available to the skilled artisan which describe protecting groups and may be useful in selecting suitable protecting groups for the atoms or functional groups to be protected. For suitable protecting groups and methods to protect compounds with suitable protecting groups, see, for example, Protective Groups in Organic Synthesis, 4th Edition, 2007, John Wiley & Sons, Inc., Hoboken, New Jersey. Examples of atoms and functional groups that may be protected with protecting groups include, but are not limited to, optionally substituted 1H-indole, preferably the nitrogen of the 1H-indole, O, S, N, OH, SH, NH₂, carbonyls such as aldehydes and ketones, ethers, esters, and amides. Examples of protecting groups include, but are not limited to, carbobenzyloxy (Cbz), p-methoxybenzyl carbonyl, BOC, Fmoc, acetyl, benzoyl, phenyl, benzyl, trityl, sulfonyls such as phenylsulfonyl, tosyl (Ts), mesyl, and trifyl; tosylate, silyl ethers such as trimethylsilyl (TMS), tert-butyldimethylsilyl (TBDMS), tri-iso-propylsilyloxymethyl (TOM), and triisopropylsilyl (TIPS) ethers; tetrahydropyranyl (THP), p-methoxyphenyl ether (PMP), p-methoxybenzyl ether (PMB), β-methoxyethoxymethyl ether (MEM), pivaloyl, thioethers, acetals, ketals, dithianes, benzyl esters, tert-butyl esters, orthoesters, and photolabile groups.

The term “the nitrogen of said 1H-indole is optionally protected with a protecting group” as used herein and hereafter refers to an optional protecting group that replaces the hydrogen of the nitrogen of 1H-indole

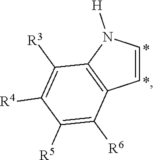

wherein R³, R⁴, R⁵, and R⁶ are as defined above and below, and therefore, said protecting group protects said nitrogen from reacting, and said 1H-indole is formed by R¹ and R², together with the carbon atoms they are attached to, of the compound of formula (I) (said 1H-indole is fused at positions 2 and 3 (indicated with asterisks) with positions 4 and 5 of the substituted 1,2,3,6-tetrahydropyridines and 2,3-dihydropyridines and, therefore, forming compounds of the invention). Similarly, “the oxygen or sulphur of said OH or SH is optionally protected with a protecting group” refers herein and hereafter to an optional protecting group that replaces the hydrogen of said OH or SH and therefore, said protecting group protects said oxygen or sulphur.

The term “activating group” as used herein and hereafter refers to a functional group of a compound that promotes a reaction to occur and/or has a positive influence of the overall reaction rate and/or have a directing effect on positional isomer of the products that are formed. Said activating group may or may not be part of the formed product, i.e. it is to be understood that the activating group may be present in the product, or the activating group may be a leaving group, or part of the leaving group, in e.g. S_{N}2, S_{N}1 and addition-elimination reactions. A compound disclosed herein may have one or more activating group(s) that may be the same or different. Examples of activating groups include, but are not limited to, sulfonyls such as phenylsulfonyl, tosyl (Ts), mesyl, and trifyl; halogen, (substituted) amino groups, amides, esters, hydroxy, alkoxy, acyloxy, thiol, alkyl, (per)haloalkyl and photolabile groups.

The term “aldehyde” as used herein and hereafter refers to a compound with an aldehyde functional group or a compound with a functional group that forms an aldehyde functional group. Examples of aldehydes include, but are not limited to, acetaldehyde, esters and anhydrides of 4-oxobutanoate and 5-oxopentanoate, such as methyl 4-oxobutanoate and methyl 5-oxopentanoate, and 1,1-diethoxyethane (acetaldehyde diethyl acetal). It is to be understood that 1,1-diethoxyethane, and other masked aldehydes useable in a method of the invention, forms acetaldehyde in situ in a ring formation reaction of a step of a method of the invention.

The term “activating group reactant” as used herein and hereafter refers to a reactant, comprising an activating group, that reacts with a compound in a reaction to form a compound or an intermediate comprising said activating group. It is to be understand that there may be different activating group reactants comprising the same activating group. The reaction may comprise one or more activating group reactant(s) that may be the same or different. Examples of activating group reactants (with examples of the corresponding activating groups in parenthesis) include, but are not limited to, sulfinic acids (sulfonyls) such as phenylsulfinic acid (phenylsulfonyl) and p-toluenesulfinic acid (tosyl); mesyl halides (mesyl) such as methanesulfonyl chloride (mesyl); trifyl azide (trifyl), trifluoromethanesulfonyl chloride (trifyl), halogens (X) such as Br₂ (Br); trifluoroacetyl chloride (TFA), and trifluoroacetic anhydride (TFA).

The term “activating agent” as used herein and hereafter refers to a substance or compound added to a reaction to cause a chemical reaction. Said activating agent may or may not be a catalyst. It is to be understood that said activating agent may or may not be consumed in the reaction. Examples of activating agents include, but are not limited to, Lewis acids such as TiCl₄, boron trifluoride, and boron trifluoride diethyl etherate.

The term “a ring formation” as used herein and hereafter refers to a reaction wherein one or more cyclic structures of a compound is formed, wherein the formed compound has more (e.g. 1, 2, 3, or 4 more) cyclic structure(s) than the starting material compound (e.g. a compound of formula (I′)). It is to be understand that the ring formation reaction may comprise one or more reactions (steps), i.e. the reaction may or may not first produce an intermediate compound, which may or may not be isolated, and said intermediate compound reacts further forming a compound with one or more cyclic structure(s), e.g. a compound of formula (I). Alternatively, the compound with one or more cyclic structure(s), e.g. a compound of formula (I), is formed by one or more ring formation reaction(s) of a one-pot reaction. Additionally, or alternatively, an intermediate compound of a short-lived intermediate is formed that produces a compound with one or more cyclic structure(s) (e.g. a compound of formula (I)).

The term “deprotection reaction” as used herein and hereafter refers to a reaction, wherein a protecting group is removed from a compound. Skilled artisans possess the knowledge and skill in the art to enable them to select suitable reactants or reagents for the deprotection reaction(s). In addition, there are a number of resources that are available to the skilled artisan which describe suitable reagents and reactants and may be useful in selecting suitable reagents and reactants for the protecting groups to be deprotected. For suitable deprotecting reactions, reactants and reagents, see, for example, Protective Groups in Organic Synthesis, 4th Edition, 2007, John Wiley & Sons, Inc., Hoboken, New Jersey. Examples of reactants and reagents usable in the deprotection reaction(s) (with examples of the protecting group to be deprotected in parenthesis) include, but are not limited to, tetra-n-butylammonium fluoride (TMS), H₂ (benzyl), bases such as NaOH (acetyl), acids such as pyridinium p-toluenesulfonate and EtOH (THP), and 2,3-dichloro-5,6-dicyano-p-benzoquinone (PMB).

The present invention provides a novel compound of formula (I)

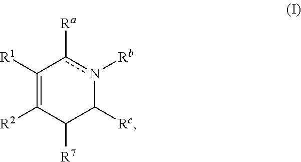

wherein the dotted line represents an optional bond;
R¹ and R², together with the carbon atoms they are attached to, form a group selected from a 1H-indole group and a benzene group, and said 1H-indole group and benzene group being optionally substituted with one to four substituent(s) each independently selected from the group consisting of R³, R⁴, R⁵, and R⁶, wherein each R³, R⁴, R⁵, and R⁶ is independently selected from the group consisting of halogen, OH, C₁₋₄-alkyl, C₁₋₃-(per)haloalkyl, C₁₋₃-alkoxy, C₁₋₃-(per)haloalkoxy;
R^{a} and R^{b}, together with the carbon atom and nitrogen atom they are attached to, form a group selected from a 5- and 6-membered cyclic amide, and
R^{c} is H; or
R^{a} is Me, and
R^{b} and R^{c}, together with the nitrogen atom and carbon atom they are attached to, form a group selected from a 5- and 6-membered cyclic amide; or
R^{a} is Me,
R^{b} is H, or R^{b} is absent when the dotted line represents a bond, and
R^{c} is H, provided that R¹ and R², together with the carbon atoms they are attached to, form said optionally substituted 1H-indole group;
R⁷ is selected from the group consisting of halogen, OH, oxo, SH, NOR⁸, C₁₋₃-(per)haloalkyl, C₁₋₃-alkoxy, C₁₋₃-(per)haloalkoxy, CN, C(O)N(R⁸)₂, and N(R⁸)₂, or
R⁷ may also be C₁₋₄-alkyl with the provisio that said 1H-indole group or benzene group is substituted with one to four substituent(s) each independently selected from the group consisting of halogen, OH, C₁₋₄-alkyl, C₁₋₃-(per)haloalkyl, C₁₋₃-alkoxy, C₁₋₃-(per)haloalkoxy, or
R⁷ may also be H provided that
when R^{a} and R^{b}, together with the carbon atom and nitrogen atom they are attached to, form a group selected from a 5- and 6-membered cyclic amide, R^{c} is H, then R⁴ and R⁵ is each independently selected from the group consisting of halogen, C₁₋₄-alkyl, C₁₋₃-(per)haloalkyl, and C₁₋₃-(per)haloalkoxy, or
when R^{a} is Me, R^{b} and R^{c}, together with the carbon atom and nitrogen atom they are attached to, form a 6-membered cyclic amide, then R¹ and R², together with the carbon atoms they are attached to, form said optionally substituted 1H-indole group, or
when R^{a} is Me, R^{b} and R^{c}, together with the carbon atom and nitrogen atom they are attached to, form a 5-membered cyclic amide, and R¹ and R², together with the carbon atoms they are attached to, form a 1H-indole group or a benzene group, then said 1H-indole group or benzene group is substituted with one to four substituent(s) each independently selected from the group consisting of halogen, OH, C₁₋₄-alkyl, C₁₋₃-(per)haloalkyl, C₁₋₃-alkoxy, and C₁₋₃-(per)haloalkoxy, or
when R^{a} is Me, R^{c} is H, R¹ and R², together with the carbon atoms they are attached to, form a substituted 1H-indole group, then R⁴ and R⁵ of said substituted 1H-indole group is each independently selected from the group consisting of halogen, C₁₋₄-alkyl, C₁₋₃-(per)haloalkyl, and C₁₋₃-(per)haloalkoxy;
each R⁸ is independently selected from the group consisting of H, C₁₋₄-alkyl, C₁₋₄-alkenyl, C₁₋₄-alkynyl, and C₁₋₃-(per)haloalkyl, or when part of any N(R⁸)₂ both R⁸ together with the nitrogen they are attached to may form a 3- to 6-membered aliphatic or aromatic heterocyclic ring comprising 1 to 3 heteroatoms each independently selected from N, O, and S;
or a stereoisomer or a pharmaceutically acceptable salt thereof.

The term “the dotted line represents an optional bond” as used herein and hereafter refers to a bond that may or may not be present. It is to be understood that when the dotted line is present then it forms, together with the single bond next to it, a double bond and therefore, the compound of formula (I) is equal to a compound of formula (II)

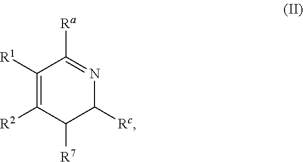

and when the dotted line is not present, the compound of formula (I) is equal to a compound of formula (III)

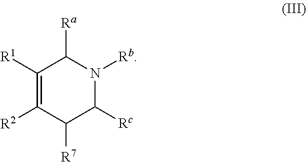

The term “1H-indole group” as used herein and hereafter refers to a 1H-indole, which is fused at positions 2 and 3 with the carbons that R¹ and R² of the compound of formula (I) are attached to. The term “benzene group” as used herein and hereafter refers to benzene, which is fused with the carbons that R¹ and R² of the compound of formula (I) are attached to. Therefore, term “R¹ and R², together with the carbon atoms they are attached to, form a group selected from a 1H-indole group and a benzene group” as used herein and hereafter refers to a 1H-indole ring and a benzene ring fused with derivatives of 1,2,3,6-tetrahydropyridine and 2,3-dihydropyridine of the invention. It is to be understood that when R¹ and R², together with the carbon atoms they are attached to, form a 1H-indole group, then a compound of formula (I) is equal to a compound of formula (IV)

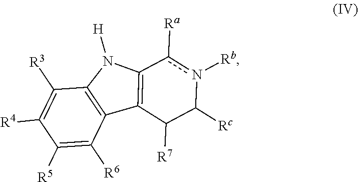

wherein R³, R⁴, R⁵, R⁶, R⁷, R⁸, R^{a}, R^{b}, R^{c}, and the dotted line are as defined above, and when R¹ and R², together with the carbon atoms they are attached to, form a benzene group, then a compound formula (I) is equal to a compound of formula (V)

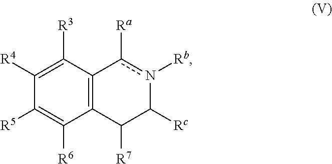

wherein R³, R⁴, R⁵, R⁶, R⁷, R⁸, R^{a}, R^{b}, R^{c}, and the dotted line are as defined above.

Accordingly, in embodiments, the compound has formula (Ia), (Ib), or (Ic)

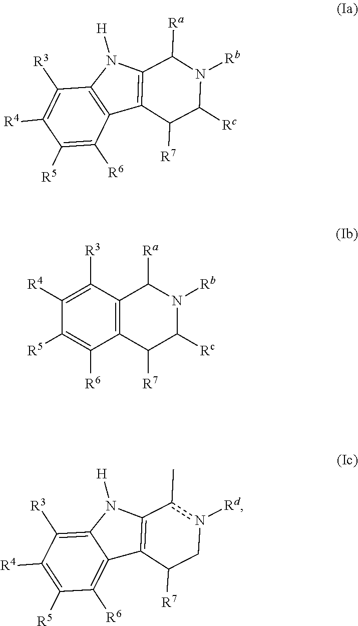

wherein
R^{a} and R^{b}, together with the carbon atom and nitrogen atom they are attached to, form a group selected from a 5- and 6-membered cyclic amide, and
R^{c} is H; or
R^{a} is Me, and
R^{b} and R^{c}, together with the nitrogen atom and carbon atom they are attached to, form a group selected from a 5- and 6-membered cyclic amide;
R^{d} is H, or R^{d} is absent when the dotted line represents a bond; and
R³, R⁴, R⁵, R⁶, R⁷, R⁸, and the dotted line are as defined above;
or a stereoisomer or a pharmaceutically acceptable salt thereof.

Furthermore, selection of the substituents R³, R⁴, R⁵, R⁶, R⁷, and R⁸ is particularly important for attaining the desired properties of the compounds of the present invention.

Additionally, or alternatively, selection of the substituents R^{a}, R^{b}, R^{c}, and R^{d} is particularly important for attaining the desired properties of the compounds of the present invention.

In embodiments, the compound has formula (Ic), (Id), (Ie), (If), or (Ig)

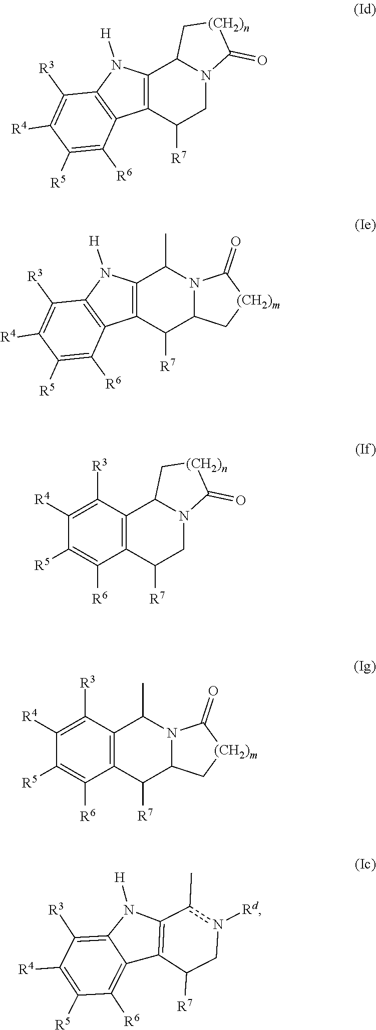

wherein
m is 1 or 2;
n is 1 or 2;
R^{d} is H, or R^{d} is absent when the dotted line represents a bond; and
the dotted line, R³, R⁴, R⁵, R⁶, R⁷, and R⁸ are as defined above;
or a stereoisomer or a pharmaceutically acceptable salt thereof.

Furthermore, selection of the substituents R³, R⁴, R⁵, R⁶, R⁷, and R⁸ is particularly important for attaining the desired properties of the compounds of the present invention.

Additionally, or alternatively, selection of m, n, and the substituents R^{a}, R^{b}, R^{c}, and R^{d} is particularly important for attaining the desired properties of the compounds of the present invention.

In embodiments, the compound has formula (Id), (Ie), (If), or (Ig)

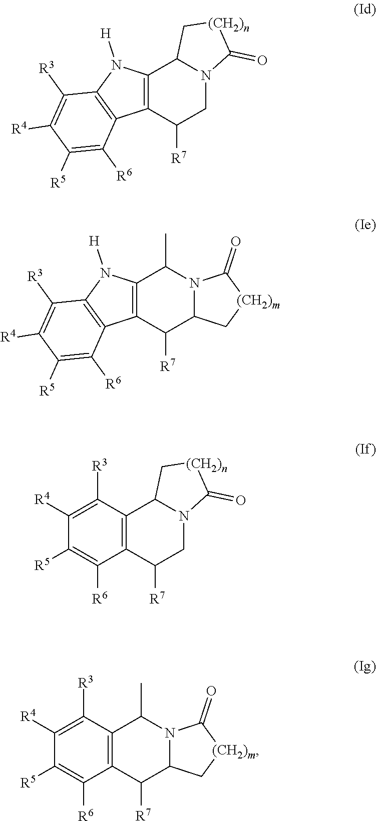

wherein
m is 1 or 2;
n is 1 or 2; and
R³, R⁴, R⁵, R⁶, R⁷, and R⁸ are as defined above;
or a stereoisomer or a pharmaceutically acceptable salt thereof.

Furthermore, selection of the substituents R³, R⁴, R⁵, R⁶, R⁷, and R⁸ is particularly important for attaining the desired properties of the compounds of the present invention. Additionally, or alternatively, selection of m, and n is particularly important for attaining the desired properties of the compounds of the present invention.

In embodiments, the compound has formula (Id), or (If), preferably (Id), wherein
n is 1 or 2, preferably 1; and
R³, R⁴, R⁵, R⁶, R⁷, and R⁸ are as defined above;
or a stereoisomer or a pharmaceutically acceptable salt thereof. In a preferred embodiment, the compound has formula (Id) and n is 1. Furthermore, selection of the substituents R³, R⁴, R⁵, R⁶, R⁷, and R⁸ is particularly important for attaining the desired properties of the compounds of the present invention.

In embodiments, the compound has formula (Ie), or (Ig), preferably (Ig), wherein
m is 1 or 2, preferably 1; and
R³, R⁴, R⁵, R⁶, R⁷, and R⁸ are as defined above;
or a stereoisomer or a pharmaceutically acceptable salt thereof. In a preferred embodiment, the compound has formula (Ig) and m is 1. Furthermore, selection of the substituents R³, R⁴, R⁵, R⁶, R⁷, and R⁸ is particularly important for attaining the desired properties of the compounds of the present invention.

In embodiments, the compound has formula (Ic), wherein
R^{d} is H, or R^{d} is absent when the dotted line represents a bond; and
the dotted line, R³, R⁴, R⁵, R⁶, R⁷, and R⁸ are as defined above;
or a stereoisomer or a pharmaceutically acceptable salt thereof.

Furthermore, selection of the substituents R³, R⁴, R⁵, R⁶, R⁷, and R⁸ is particularly important for attaining the desired properties of the compounds of the present invention.

In embodiments, the compound has formula (I), wherein
R¹ and R², together with the carbon atoms they are attached to, form a group selected from a 1H-indole group, and said 1H-indole group being optionally substituted with one to four substituent(s) each independently selected from the group consisting of R³, R⁴, R⁵, and R⁶, wherein each R³, R⁴, R⁵, and R⁶ is independently selected from the group consisting of halogen, OH, C₁₋₄-alkyl, C₁₋₃-(per)haloalkyl, C₁₋₃-alkoxy, C₁₋₃-(per)haloalkoxy;
R^{a} is Me;
R^{b} is H, or R^{b} is absent when the dotted line represents a bond, preferably R^{b} is H;
R^{c} is H; and
the dotted line, R⁷, and R⁸ are as defined above;
or a stereoisomer or a pharmaceutically acceptable salt thereof.

Furthermore, selection of the substituents R³, R⁴, R⁵, R⁶, R⁷, and R⁸ is particularly important for attaining the desired properties of the compounds of the present invention.

In embodiments, the compound has formula (I), (Ic), (Id), (Ie), (If), or (Ig), wherein
R³ and R⁶ are each independently selected from the group consisting of H, halogen, C₁₋₄-alkyl, C₁₋₃-(per)haloalkyl, and C₁₋₃-(per)haloalkoxy, preferably each independently selected from H and F;
R⁴ and R⁵ are both halogen, preferably F, or one of R⁴ and R⁵ is halogen, preferably F, and the other is C₁₋₄-alkyl, C₁₋₃-(per)haloalkyl, or C₁₋₃-(per)haloalkoxy, preferably methoxy, methyl, or trifluoromethyl;
R⁷ is selected from the group consisting of H, halogen, OH, oxo, NOR⁸, C₁₋₄-alkyl, C₁₋₃-(per)haloalkyl, C₁₋₃-alkoxy, and C₁₋₃-(per)haloalkoxy, preferably H, F, OH, or methoxy, most preferably F; and
the dotted line, R¹, R², R^{a}, R^{b}, R^{c}, R^{d}, and R⁸ are as defined above;
or a stereoisomer or a pharmaceutically acceptable salt thereof.

In embodiments, the compound has formula (I), (Ic), (Id), (Ie), (If), or (Ig), wherein
R³ and R⁶ are each independently selected from the group consisting of H, halogen, C₁₋₄-alkyl, C₁₋₃-(per)haloalkyl, and C₁₋₃-(per)haloalkoxy, preferably each independently selected from H and F, most preferably H;
R⁴ and R⁵ are both F, or one of R⁴ and R⁵ is F and the other is C₁₋₄-alkyl or C₁₋₃-(per)haloalkyl, preferably both are F;
R⁷ is selected from the group consisting of H, halogen, OH, oxo, NOR⁸, C₁₋₄-alkyl, C₁₋₃-(per)haloalkyl, C₁₋₃-alkoxy, and C₁₋₃-(per)haloalkoxy, preferably H, F, OH, or methoxy, most preferably F; and
the dotted line, R¹, R², R^{a}, R^{b}, R^{c}, R^{d}, and R⁸ are as defined above;
or a stereoisomer or a pharmaceutically acceptable salt thereof.

In embodiments, the compound has formula (I), (Ic), (Id), (Ie), (If), or (Ig), wherein
R³ and R⁶ are each independently selected from the group consisting of H, halogen, C₁₋₄-alkyl, C₁₋₃-(per)haloalkyl, and C₁₋₃-(per)haloalkoxy, preferably each independently selected from H and F, most preferably H;
R⁴ and R⁵ are both F, or one of R⁴ and R⁵ is halogen, preferably F, and the other is H, C₁₋₄-alkyl or C₁₋₃-(per)haloalkyl, preferably one of R⁴ and R⁵ is halogen and the other is H;
R⁷ is selected from the group consisting of halogen, OH, C₁₋₃-alkoxy, C₁₋₃-(per)haloalkoxy, preferably F, OH, methoxy, and ethoxy, most preferably F; and
the dotted line, R¹, R², R^{a}, R^{b}, R^{c}, R^{d}, and R⁸ are as defined above;
or a stereoisomer or a pharmaceutically acceptable salt thereof.

In embodiments, R⁴ and R⁵ are both halogen, preferably F. In another embodiments, R⁴ is H, R⁵ is halogen, preferably F, and R⁷ is selected from the group consisting of halogen, preferably F and OH. In another embodiments, R⁴ is halogen, preferably F, R⁵ is H, and R⁷ is selected from the group consisting of halogen, preferably F and OH.

In embodiments, the compound has formula (I), (Ic), (Id), (Ie), (If), or (Ig), wherein
R³ and R⁶ are each independently selected from H and halogen, preferably each independently selected from H and F;
R⁴ and R⁵ are both F, or one of R⁴ and R⁵ is F and the other is C₁₋₄-alkyl or C₁₋₃-(per)haloalkyl, preferably both are F; and
R⁷ is selected from the group consisting of H, F, OH, C₁₋₄-alkyl, and methoxy, preferably H, F, OH, or methoxy, more preferably H, or F, most preferably F; and
the dotted line, R¹, R², R^{a}, R^{b}, R^{c}, R^{d}, and R⁸ are as defined above;
or a stereoisomer or a pharmaceutically acceptable salt thereof.

In embodiments, the compound has formula (Ic), (Id), (Ie), (If), or (Ig), wherein
m is 1 or 2, preferably 1;
n is 1 or 2, preferably 1;
R³ and R⁶ are each independently selected from the group consisting of H, halogen, OH, C₁₋₄-alkyl, C₁₋₃-(per)haloalkyl, C₁₋₃-alkoxy, C1-3-(per)haloalkoxy, preferably each independently selected from H, F, methoxy, ethoxy, more preferably each independently selected from H and F;
R⁴ and R⁵ are both halogen, preferably F, or one of R⁴ and R⁵ is halogen, preferably F, and the other is H, C₁₋₃-alkoxy, C₁₋₄-alkyl, C₁₋₃-(per)haloalkyl, or C₁₋₃-(per)haloalkoxy, preferably H, methoxy, methyl, or trifluoromethyl, most preferably H;
R⁷ is selected from the group consisting of halogen, OH, oxo, NOR⁸, C₁₋₄-alkyl, C₁₋₃-(per)haloalkyl, C₁₋₃-alkoxy, and C₁₋₃-(per)haloalkoxy, preferably H, F, OH, or methoxy, most preferably F; and
R⁸, R^{d}, and R⁸ are as defined above;
or a stereoisomer or a pharmaceutically acceptable salt thereof.

In embodiments, the compound has formula (Ie), or (Ig), wherein
m is 1;
R³ and R⁶ are each independently selected from the group consisting of H, halogen, C₁₋₄-alkyl, C₁₋₃-(per)haloalkyl, and C₁₋₃-(per)haloalkoxy, preferably each independently selected from H and F;
R⁴ and R⁵ are both halogen, preferably F, or one of R⁴ and R⁵ is halogen, preferably F, and the other is H, C₁₋₃-alkoxy, C₁₋₄-alkyl, C₁₋₃-(per)haloalkyl, or C₁₋₃-(per)haloalkoxy, preferably H, methoxy, methyl, or trifluoromethyl, most preferably H;
R⁷ is selected from the group consisting of H, halogen, OH, oxo, NOR⁸, C₁₋₄-alkyl, C₁₋₃-(per)haloalkyl, C₁₋₃-alkoxy, and C₁₋₃-(per)haloalkoxy, preferably H, F, OH, or methoxy, most preferably F; and
R⁸ are as defined above;
or a stereoisomer or a pharmaceutically acceptable salt thereof.

In embodiments, the compound has formula (Id), or (If), wherein
n is 1 or 2, preferably 1;
R³ and R⁶ are each independently selected from the group consisting of H, halogen, C₁₋₄-alkyl, C₁₋₃-(per)haloalkyl, and C₁₋₃-(per)haloalkoxy, preferably each independently selected from H and F;
R⁴ and R⁵ are both halogen, preferably F, or one of R⁴ and R⁵ is halogen, preferably F, and the other is C₁₋₄-alkyl, C₁₋₃-(per)haloalkyl, or C₁₋₃-(per)haloalkoxy, preferably methyl, trifluoromethyl, or trifluoromethoxy, most preferably trifluoromethyl;
R⁷ is selected from the group consisting of H, halogen, OH, oxo, NOR⁸, C₁₋₄-alkyl, C₁₋₃-(per)haloalkyl, C₁₋₃-alkoxy, and C₁₋₃-(per)haloalkoxy, preferably H, F, OH, or methoxy, most preferably F; and
R⁸ is as defined above;
or a stereoisomer or a pharmaceutically acceptable salt thereof.

In embodiments, the compound has formula (Ic), wherein
R³ and R⁶ are each independently selected from the group consisting of H, halogen, C₁₋₄-alkyl, C₁₋₃-(per)haloalkyl, and C₁₋₃-(per)haloalkoxy, preferably each independently selected from H and F;
R⁴ and R⁵ are both halogen, preferably F, or one of R⁴ and R⁵ is halogen, preferably F, and the other is C₁₋₄-alkyl, C₁₋₃-(per)haloalkyl, or C₁₋₃-(per)haloalkoxy, preferably methyl, trifluoromethyl, or trifluoromethoxy, most preferably trifluoromethyl;
R⁷ is selected from the group consisting of H, halogen, OH, oxo, NOR⁸, C₁₋₄-alkyl, C₁₋₃-(per)haloalkyl, C₁₋₃-alkoxy, and C₁₋₃-(per)haloalkoxy, preferably H, F, OH, or methoxy, most preferably F;
R^{b} is H, or R^{b} is absent when the dotted line represents a bond, preferably R^{b} is H; and
R⁸ is as defined above;
or a stereoisomer or a pharmaceutically acceptable salt thereof.

In embodiments, the compound has formula (Ic), wherein
R³ and R⁶ are each independently selected from the group consisting of H, halogen, C₁₋₄-alkyl, C₁₋₃-(per)haloalkyl, and C₁₋₃-(per)haloalkoxy, preferably each independently selected from H and F;
R⁴ and R⁵ are both halogen, preferably F, or one of R⁴ and R⁵ is halogen, preferably F, and the other is H, C₁₋₄-alkyl, C₁₋₃-(per)haloalkyl, or C₁₋₃-(per)haloalkoxy, preferably H, methyl, trifluoromethyl, or trifluoromethoxy, most preferably H;
R⁷ is selected from the group consisting of halogen, OH, oxo, NOR⁸, C₁₋₃-alkoxy, and C₁₋₃-(per)haloalkoxy, preferably H, F, OH, or methoxy, most preferably F;
R^{b} is H, or R^{b} is absent when the dotted line represents a bond, preferably R^{b} is H; and
R⁸ is as defined above;
or a stereoisomer or a pharmaceutically acceptable salt thereof.

In embodiments, the compound has formula (I), (Ic), (Id), (Ie), (If), or (Ig), wherein
R³, R⁶, and R⁷ are H;
R⁴ and R⁵ are F; and
the dotted line, R¹, R², R^{a}, R^{b}, R^{c}, and R^{d}, are as defined above;
or a stereoisomer or a pharmaceutically acceptable salt thereof.

In preferred embodiments, the compound has formula (Id), (Ie), (If), or (Ig), more preferably (Ie), or (Ig), most preferably (Ig), wherein
R³, R⁶, and R⁷ are H; and
R⁴ and R⁵ are F;
or a stereoisomer or a pharmaceutically acceptable salt thereof.

10. In embodiments, the compound has formula (I), (Ic), (Id), (Ie), (If), or (Ig), wherein
R³ and R⁶ are H;
R⁴, R⁵ and R⁷ are F; and
the dotted line, R¹, R², R^{a}, R^{b}, R^{c}, and R^{d}, are as defined above;
   or a stereoisomer or a pharmaceutically acceptable salt thereof.

In embodiments, the compound has formula (I), (Id), (Ie), (If), or (Ig), wherein
R³ and R⁶ are H;
R⁴, R⁵ and R⁷ are F;
R^{a} is Me;
R^{b} and R^{c}, together with the nitrogen atom and carbon atom they are attached to, form a group selected from a 5- and 6-membered cyclic amide; and
R¹, and R² are as defined above;
   or a stereoisomer or a pharmaceutically acceptable salt thereof.

In preferred embodiments, the compound has formula (Id), (Ie), (If), or (Ig), more preferably (Ie), or (Ig), most preferably (Ig), wherein
R³ and R⁶ are H;
R⁴, R⁵ and R⁷ are F;
m is 1 or 2, preferably 1; and
n is 1 or 2, preferably 1;
   or a stereoisomer or a pharmaceutically acceptable salt thereof.

In a preferred embodiment, the compound has formula (Ig), wherein
m is 1;
R³ and R⁶ are H; and
R⁴, R⁵ and R⁷ are F;
   or a stereoisomer or a pharmaceutically acceptable salt thereof.

In embodiments, the compound of formula (I), (Ia), (Ib), (Ic), (Id), (Ie), (If), or (Ig) is selected from the group consisting of:
5-Fluoro-1-methyl-2,3,4,9-tetrahydro-1H-pyrido[3,4-b]indole;
6-Fluoro-1-methyl-2,3,4,9-tetrahydro-1H-pyrido[3,4-b]indole;
4,6-Difluoro-1-methyl-2,3,4,9-tetrahydro-1H-pyrido[3,4-b]indole;
7-Fluoro-1H,2H,3H,4H,6H,7H,12H,12bH-indolo[2,3-a]quinolizin-4-one; 7,8,9,10-Tetrafluoro-1H,2H,3H,4H,6H,7H,12H,12bH-indolo[2,3-a]quinolizin-4-one;
(7R,12bS)-7,8,9,10-Tetrafluoro-1H,2H,3H,4H,6H,7H,12H,12bH-indolo[2,3-a]quinolizin-4-one;
(7R,12bR)-7,8,9,10-Tetrafluoro-1H,2H,3H,4H,6H,7H,12H,12bH-indolo[2,3-a]quinolizin-4-one;
(7S,12bR)-7,8,9,10-Tetrafluoro-1H,2H,3H,4H,6H,7H,12H,12bH-indolo[2,3-a]quinolizin-4-one;
7,9-Difluoro-1H,2H,3H,4H,6H,7H,12H,12bH-indolo[2,3-a]quinolizin-4-one;
7,8,10-Trifluoro-1H,2H,3H,4H,6H,7H,12H,12bH-indolo[2,3-a]quinolizin-4-one;
4,5,6,7-Tetrafluoro-1-methyl-2,3,4,9-tetrahydro-1H-pyrido[3,4-b]indole;
5,6-difluoro-1-methyl-4,9-dihydro-3H-pyrido[3,4-b]indole;
5,6,7-trifluoro-1-methyl-4,9-dihydro-3H-pyrido[3,4-b]indole;
4,5,6,7-tetrafluoro-1-methyl-4,9-dihydro-3H-pyrido[3,4-b]indole;
7,8,10-Trifluoro-5-methyl-1,5,10,10a-tetrahydropyrrolo[1,2-b]isoquinolin-3(2H)-one;
(5S,10R,10aR)-7,8,10-Trifluoro-5-methyl-1,5,10,10a-tetrahydropyrrolo[1,2-b]isoquinolin-3(2H)-one;
(5S,10R,10aS)-7,8,10-Trifluoro-5-methyl-1,5,10,10a-tetrahydropyrrolo[1,2-b]isoquinolin-3(2H)-one;
(5S,10S,10aR)-7,8,10-Trifluoro-5-methyl-1,5,10,10a-tetrahydropyrrolo[1,2-b]isoquinolin-3(2H)-one;
7,10-Difluoro-5-methyl-1,5,10,10a-tetrahydropyrrolo[1,2-b]isoquinolin-3(2H)-one;
9,10-Difluoro-5-methyl-1,5,10,10a-tetrahydropyrrolo[1,2-b]isoquinolin-3(2H)-one;
7,8-Difluoro-5-methyl-1,5,10,10a-tetrahydropyrrolo[1,2-b]isoquinolin-3(2H)-one;
(5R,11S)-10,11-Difluoro-5-methyl-1,2,5,6,11,11a-hexahydro-3H-indolizino[6,7-b]indol-3-one;
(6R,12S)-12-Fluoro-6-methyl-6,9,10,11,11a,12-hexahydroindolo[3,2-b]quinolizin-8(5H)-one;
(5R,11S)-11-Fluoro-5-methyl-1,2,5,6,11,11a-hexahydro-3H-indolizino[6,7-b]indol-3-one;
(6R,12S)-1,12-Difluoro-6-methyl-6,9,10,11,11a,12-hexahydroindolo[3,2-b]quinolizin-8(5H)-one;
(5R)-9-Fluoro-5-methyl-1,2,5,6,11,11a-hexahydro-3H-indolizino[6,7-b]indol-3-one;
(6R)-2-Fluoro-6-methyl-6,9,10,11,11a,12-hexahydroindolo[3,2-b]quinolizin-8(5H)-one;
(4S)-4,6-difluoro-1-methyl-4,9-dihydro-3H-pyrido[3,4-b]indole;
(5S,6S,10bS)-6,9-difluoro-5-methyl-1,5,6,10b-tetrahydropyrrolo[2,1-a]isoquinolin-3(2H)-one;
(5R,6R,10bS)-6,9-difluoro-5-methyl-1,5,6,10b-tetrahydropyrrolo[2,1-a]isoquinolin-3(2H)-one;
(5R,6S,10bR)-6,8,9-trifluoro-5-methyl-1,5,6,10b-tetrahydropyrrolo[2,1-a]isoquinolin-3(2H)-one;
(12bS)-8,9-difluoro-1H,2H,3H,4H,6H,7H,12H,12bH-indolo[2,3-a]quinolizin-4-one;
(7R,12bS)-7,8,9-trifluoro-1H,2H,3H,4H,6H,7H,12H,12bH-indolo[2,3-a]quinolizin-4-one;
(7S,12bR)-7,8,9-trifluoro-1H,2H,3H,4H,6H,7H,12H,12bH-indolo[2,3-a]quinolizin-4-one;
(7R,12bR)-7,8,9-trifluoro-1H,2H,3H,4H,6H,7H,12H,12bH-indolo[2,3-a]quinolizin-4-one;
(12bS)-9,10-difluoro-1H,2H,3H,4H,6H,7H,12H,12bH-indolo[2,3-a]quinolizin-4-one;
(7R,12bR)-7,8,9-trifluoro-1H,2H,3H,4H,6H,7H,12H,12bH-indolo[2,3-a]quinolizin-4-one;
(7S,12bS)-7,8,9-trifluoro-1H,2H,3H,4H,6H,7H,12H,12bH-indolo[2,3-a]quinolizin-4-one;
(5S,6R,10bS)-6,8-difluoro-5-methyl-1,5,6,10b-tetrahydropyrrolo[2,1-a]isoquinolin-3(2H)-one;
(5S,10S,10aR)-8,10-difluoro-5-methyl-1,5,10,10a-tetrahydropyrrolo[1,2-b]isoquinolin-3(2H)-one;
(1R,4R)-4,6,7-trifluoro-1-methyl-2,3,4,9-tetrahydro-1H-pyrido[3,4-b]indole;
(7R,12bS)-7,9,10-trifluoro-1H,2H,3H,4H,6H,7H,12H,12bH-indolo[2,3-a]quinolizin-4-one;
(7R,12bR)-7,8-difluoro-1H,2H,3H,4H,6H,7H,12H,12bH-indolo[2,3-a]quinolizin-4-one;
(5R,6R,10bS)-6,9-difluoro-5-methyl-1,5,6,10b-tetrahydropyrrolo[2,1-a]isoquinolin-3(2H)-one; and
6-fluoro-1-methyl-4,9-dihydro-3H-pyrido[3,4-b]indole;

a stereoisomer or a pharmaceutically acceptable salt thereof.

In one aspect of the present invention is provided a pharmaceutical composition comprising an effective amount of one or more compounds of formula (I), or a stereoisomer or a pharmaceutically acceptable salt thereof, together with one or more pharmaceutically acceptable excipient(s).

Additionally, or alternatively, to pharmaceutically acceptable excipient(s), pharmaceutical compositions of the present disclosure comprise an effective amount of one or more compounds of formula (I), or a stereoisomer or a pharmaceutically acceptable salt thereof, in combination with one or more pharmaceutically acceptable carrier(s). Therefore, in embodiments, pharmaceutical compositions comprise an effective amount of one or more compounds of formula (I), or a stereoisomer or a pharmaceutically acceptable salt thereof, together with one or more pharmaceutically acceptable excipient(s) and/or one or more pharmaceutically acceptable carrier(s), or any combination thereof. Preferably, pharmaceutical compositions comprise one compound of formula (I), or a stereoisomer or a pharmaceutically acceptable salt thereof, preferably a pharmaceutically acceptable salt thereof, in combination with one or more pharmaceutically acceptable excipient(s) and one pharmaceutically acceptable carrier.

Additionally, or alternatively, to pharmaceutically acceptable excipient(s) and/or pharmaceutically acceptable carrier(s), pharmaceutical compositions of the present disclosure comprise an effective amount of one or more compounds of formula (I), or a stereoisomer or a pharmaceutically acceptable salt thereof, in combination with one or more other active ingredient(s). Therefore, in embodiments, pharmaceutical compositions comprise an effective amount of one or more compounds of formula (I), or a stereoisomer or a pharmaceutically acceptable salt thereof, together with one or more pharmaceutically acceptable excipient(s) and/or one or more pharmaceutically acceptable carrier(s) and/or one or more other active ingredient(s), or any combination thereof.

In embodiments, pharmaceutical compositions consist an effective amount of one or more compounds of formula (I), or a stereoisomer or a pharmaceutically acceptable salt thereof, preferably a pharmaceutically acceptable salt thereof, together with one or more pharmaceutically acceptable excipient(s), preferably 1, 2, or 3 pharmaceutically acceptable excipient(s), more preferably 1 pharmaceutically acceptable excipient.

In embodiments, pharmaceutical compositions consist an effective amount of one or more compounds of formula (I), preferably an effective amount of 1 or 2 compounds of formula (I), more preferably 1 compound of formula (I), or a stereoisomer or a pharmaceutically acceptable salt thereof, preferably a pharmaceutically acceptable salt thereof, together with one or more pharmaceutically acceptable excipient(s), preferably 1, 2, or 3 pharmaceutically acceptable excipient(s), and/or one or more pharmaceutically acceptable carrier(s), preferably 1, 2, or 3 pharmaceutically acceptable carrier(s), more preferably 1 pharmaceutically acceptable carrier.

In embodiments, pharmaceutical compositions consist an effective amount of one or more compounds of formula (I), preferably an effective amount of 1 or 2 compounds of formula (I), more preferably 1 compound of formula (I), or a stereoisomer or a pharmaceutically acceptable salt thereof, preferably a pharmaceutically acceptable salt thereof, together with one or more pharmaceutically acceptable excipient(s), preferably 1, 2, or 3 pharmaceutically acceptable excipient(s), and/or one or more pharmaceutically acceptable carrier(s), preferably 1, 2, or 3 pharmaceutically acceptable carrier(s), more preferably 1 pharmaceutically acceptable carrier, and/or one or more other active ingredient(s), preferably one other active ingredient.

In one aspect of the present invention is provided a compound of formula (I), or a stereoisomer or pharmaceutically acceptable salt thereof, for use as a medicament.

In one aspect of the present invention is provided a compound of formula (I), or a stereoisomer or pharmaceutically acceptable salt thereof, for use in treatment or prevention of CNS related diseases or conditions.

In embodiments of the present invention is provided a compound of formula (I), or a stereoisomer or pharmaceutically acceptable salt thereof, for use in the treatment or prevention of a disease or condition selected from the group consisting of Alzheimer's disease, Parkinson's disease, depression, anxiety, hyperactivity, narcolepsy, drug addiction, alcoholism, anorexia, bulimia, and mitochondrial disease.

In one aspect of the present invention is provided methods for the preparation of a compound of formula (I), or pharmaceutically acceptable salt or a stereoisomer thereof, comprising the steps of:
providing a compound of formula (I′)

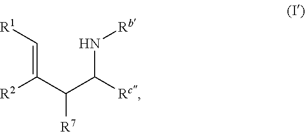

wherein R¹, R², and R⁷ are as defined herein and hereafter,
wherein when R¹ and R², together with the carbon atoms they are attached to, form a 1H-indole group, then the nitrogen of said 1H-indole group is optionally protected with a protecting group,
wherein when R⁷ is OH or SH, then the oxygen or sulphur of said OH or SH is optionally protected with a protecting group,
R^{b′} and R^{c″}, together with the carbon atom and nitrogen atom they are attached to, form a group selected from a 5- and 6-membered cyclic amide, or
R^{b′} is H or activating group, and R^{c″} is H;
reacting said compound of formula (I′) with an aldehyde, optionally in the presence of one or more activating group reactant(s), which/that optionally together with one or more activating agent(s) facilitate(s) a ring formation;
optionally performing one or more deprotection reaction(s);
to obtain the compound of formula (I)

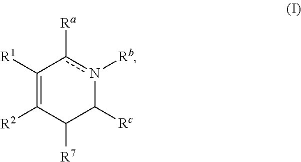

wherein R¹, R², R⁷, R^{a}, R^{b}, R^{c}, and the dotted line are as defined herein and hereafter; and
optionally converting the compound of formula (I) to a pharmaceutically acceptable salt thereof.

Additionally, or alternatively, to converting the compound of formula (I) to a pharmaceutically acceptable salt thereof, the method for the preparation of a compound of formula (I), or pharmaceutically acceptable salt or a stereoisomer thereof, further comprises the step of:

converting the compound of formula (I) to another stereoisomer thereof, wherein said step is performed before or after the step of optionally converting the compound of formula (I) to a pharmaceutically acceptable salt thereof.

In embodiments, the method for the preparation of a compound of formula (I), or pharmaceutically acceptable salt or a stereoisomer thereof, one or more activating group reactant(s), preferably one activating group reactant, which/that together with optionally one or more activating agent(s), preferably one activating agent(s), facilitate(s) said ring formation.

In embodiments, in the method for the preparation of a compound of formula (I), or pharmaceutically acceptable salt or a stereoisomer thereof, one or more activating group reactant(s), preferably one activating group reactant, which/that together with one or more activating agent(s), preferably one activating group reactant, facilitate said ring formation.

In embodiments, in the method for the preparation of a compound of formula (I), or pharmaceutically acceptable salt or a stereoisomer thereof, the aldehyde is selected from the group consisting of acetaldehyde, methyl 4-oxobutanoate, methyl 5-oxopentanoate, and 1,1-diethoxyethane.

In embodiments, in the method for the preparation of a compound of formula (I), or pharmaceutically acceptable salt or a stereoisomer thereof, the step of reacting compound of formula (I′) with an aldehyde optionally further comprises one or more aldehydes, and optionally of one or more steps of separating and/or purifying one or more compound(s) of formula (I).

The compounds of the invention can be synthesized using well documented reactions and commercially available starting materials. We have explored a series of new TIQ analogues primarily targeted to mimic the actions of 1MeTIQ and also SAL to some extent. These novel compounds can be used to achieve many desirable pharmacological responses. Fluorination can alter the bond strength, lipophilicity, conformation, electrostatic potential, dipoles, and pKa.

Substitution, especially fluorination, at the position of metabolic attack—mainly at positions corresponding to substituents R⁴, R⁵, and R⁷ of compounds having formula (Ia), (Ib), and (Ic)—is used to alter the route and rate of metabolic degradation. Fluorination may also alter the tissue distribution, pharmacodynamics, and toxicology of the compound. It can be generalized that replacing hydrogen with fluorine causes minimal steric effects at the receptor.

By replacing both of the catechol hydroxyls of SAL, especially with fluorine, a better targeting of the drug distribution and smaller active dosages are achieved. Unlike SAL, the compounds of the present invention are actively transported over the blood brain barrier by organic cation transporters and are concentrated in the brain. Most of the compounds of the present invention cannot be oxidized to form epoxides thereby making them less prone to cause oxidative stress. The compounds of the invention are associated with neuroprotective and neuroregenerative properties instead of neurotoxicity or neurodegeneration, which is the case with SAL. Further, the novel compounds are better in mimicking desirable effects of SAL and treating alcoholism and Parkinson's disease than e.g. 6-monofluorinated TIQ.

The novel compounds according to the invention show structural similarities to 1MeTIQ. Thus, the novel compounds can be used, in addition to the treatment of e.g. Alzheimer's disease and Parkinson's disease, to treat or prevent addictions in general—from alcohol to cocaine and heroin. Number of positive pharmacological responses can be achieved simultaneously. While decreasing the tendency to relapse and likelihood of developing an addiction, these compounds can act as general mood stabilizers and general neuroprotectants possessing remarkable antiparkinsonian and antiepileptic character.

The compounds exhibit many pharmacological responses, such as
prolonging the duration of morphine without enhancing the peak action;
antagonizing the development of morphine tolerance;
reducing the naloxone-precipitated withdrawal symptoms;
inhibiting the reinstatement of cocaine self-administration;
attenuating cravings, inhibiting the activity of monoamine-oxidases (MAOs);
inhibiting the activity of acetylcholinesterase (ACE);
affecting the activity of one or more of trace amine receptor, dopamine receptors and transporter, serotonine receptors and transporter, serotonine acyl and methyltransferases, norepinephrine transporter, monoamino-oxidases, catecholine-O-methyltransferase, and shifting metabolism towards it; adrenergic receptors, tyrosine hydroxylase, histamine receptors, orexin receptors, NMDA-receptors, sigma 1-receptor, muscarinic and nicotinic acetylcholine receptors, opioid receptors, neuropeptide receptors, melanocortin receptors (excluding MC3R), neurokinin receptors, corticotropin release;
neuroprotection;
shifting the catabolism of catecholamine neurotransmitters towards catechol-O-methyl transferase (COMT)-dependent methylation;
inhibition or enhancement of the release of prolactin;
releasing norepinephrine;
inducing or inhibiting neuron related apoptosis and/or necrosis;
abolishing cocaine induced inhibition of noradrenalin metabolism;
ameliorating mitochondrial disease/dysfunction.

Further, the invention may also be for use in the treatment or prevention of a disease or condition associated with HIV transcriptase.

Furthermore, compounds of formula (I) may be used as synthesis intermediates for the preparation of other compounds, in particular of other pharmaceutically active compositions, which are obtainable from compounds of formula (I) and, for example by introduction of substituents or modification of functional moieties.

The compounds and pharmaceutical compositions of the invention may also be useful in medical devices and medical kits.

### General Preparation Methods

The compounds according to the present invention may be prepared by processes known per se as follows.

The following examples illustrate the preparation of compounds of formula (I).

### General Procedure for the Preparation of Optionally Substituted Phenethylamines, General Procedure A

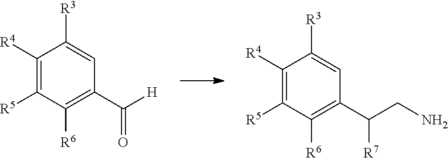

Step 1. 1.0 mol eq. optionally subst. benzaldehyde, 1.2 mol eq. nitromethane, 0.47 mol eq. ammonium acetate and 0.35 mol eq. glacial acetic acid (GAA) was sonicated (40 kHz) at RT for 3 h. After removal of nitromethane, partition between dichloromethane and water then brine gave a crude product which was recrystallized from aq, (m)ethanol or AcOH; or
1.0 mol eq. optionally subst. benzaldehyde, 1.2 mol eq. nitromethane and 0.1 mol eq. cyclohexylamine was mixed and kept in dark for 4 weeks, or until H₂O formation ceased. The crude product was ground, washed with brine and recrystallized from aq. (m)ethanol or AcOH; or
in the case of R⁷-halogen substitution (preferably fluorine), the synthesis proceeds via nitroalcohol intermediate, otherwise skip to step 4:
1.0 mol eq. optionally subst. benzaldehyde, with 1.0 mol eq. of triethylamine and 1.2 mol eq. nitromethane was stirred in methanol at −12° C. for 2.5 h, and the amine quenched with 1.0 mol eq. of GAA while still freezing cold. Most of the solvent was stripped under vacuum, and the remains were dissolved in dichloromethane (DCM) and washed two times with water and once with brine. The DCM was stripped, leaving behind the crude nitroalcohol.

Step 2. (in the case of R⁷-halogen substitution) Sulfonating the aliphatic OH-group to appropriate sulfonyl ester by stirring 1.0 mol eq. of subst. nitroalcohol in DCM with 1.2 mol eq. of triethylamine (or using pyridine for the solvent) and adding slowly 1.1 mol eq. of p-toluenesulfonyl chloride maintaining the temperature at −5° C. until conversion was complete. The product was washed several times with brine, dried over anhydrous MgSO₄ and concentrated in vacuo.

Step 3. (in the case of R⁷-halogen substitution) Modified Finkelstein reaction to the sulfonate ester with potassium halide (in this case KF) proceeded by dissolving 1 mol eq. of sulfonyl-intermediate from the step 2 with 6 ml of acetonitrile per gram of substrate, 0.5 mol eq. of 1-butyl-3-methylimidazolium tetrafluoroborate and 5 mol eq. of H₂O which after 1.05 mol eq. amount of KF was added and the solution was mixed and sonicated at RT for 180 min or until TLC showed completion. The R⁷-halogen substituted compounds were then extracted with DCM and washed several times with brine, dried over anhydrous MgSO₄ and concentrated in vacuo prior proceeding.

Step 4. In the case of protected R⁷—OH groups, the synthesis proceeds via nitroalcohol intermediate:

Protecting the aliphatic OH-group with appropriate protecting group according to procedures well known in the art, e.g. as a silyl ether by stirring 1.0 mol eq. of optionally subst. nitroalcohol in DCM with 1.2 mol eq. of pyridine (or using pyridine for the solvent) and adding slowly 1.1 mol eq. of trimethylsilyl chloride maintaining the temperature at 0° C. until conversion was complete. The product was washed several times with brine, dried over anhydrous MgSO₄ and concentrated in vacuo; or
in the case of R⁷-alkoxy or -(per)haloalkoxy substitution, the synthesis proceeds via nitroalcohol intermediate:
O-Alkylating the aliphatic OH-group with appropriate alkyl- or (per)haloalkyl-group according to procedures well known in the art, e.g. as a O-methyl ether by stirring 1.0 mol eq. of optionally subst. nitroalcohol in DCM (or THF) with 1.05 mol eq. of diazomethane and 1.0 mol eq. of boron trifluoride diethyl etherate at 0° C. until conversion was complete. The product was washed several times with brine, dried over anhydrous MgSO₄ and concentrated in vacuo; or
skip to step 5.

Step 5. Reduction of the possible C═C bond and nitro-group to the amino-group according to procedures well known in the art, e.g. with catalytic hydrogenation using platinum(IV) oxide (PtO₂), Raney nickel and/or Platinum on carbon (Pt/C).

### General Procedure for the Preparation of Optionally Substituted 2-(1H-indol-3-yl)ethan-1-amines, General Procedure B

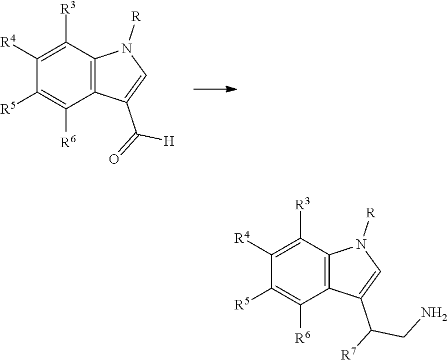

General procedure A was followed except that an optionally subst. 1H-indole-3-carbaldehyde or N-protected 1H-indole-3-carboxaldehyde (e.g. 1-benzyl-1H-indole-3-carboxaldehyde or 1-(triisopropylsilyl)-1H-indole-3-carbaldehyde) was used instead of the optionally subst. benzaldehyde.

### General Procedure for the Preparation of Optionally Substituted N-Tosyl-Phenethylamines, General Procedure C

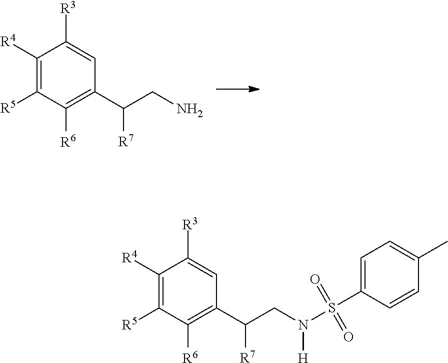

To 1.0 mol eq. of the product from step 5 of general procedure A in DCM with 1.2 mol eq. of triethylamine (or using pyridine for the solvent) was added slowly 1.1 mol eq. of p-toluenesulfonyl chloride maintaining the temperature at −5° C. until conversion was complete. The product was washed several times with brine, dried over anhydrous MgSO₄ and concentrated in vacuo.

### General Procedure for the Preparation of Substituted N-tosyl-2-(1H-indol-3-yl)ethylamines, General Procedure C′

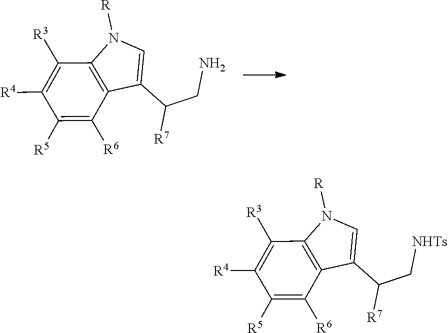

To 1.0 mol eq. of the product of general procedure B in DCM with 1.2 mol eq. of triethylamine (or using pyridine for the solvent) was added slowly 1.1 mol eq. of p-toluenesulfonyl chloride maintaining the temperature at −5° C. until conversion was complete. The product was washed several times with brine, dried over anhydrous MgSO₄ and concentrated in vacuo.

### General Procedure for the Preparation of Optionally Substituted 5-methyl-1,5,10,10a-tetrahydropyrrolo[1,2-b]isoquinolin-3(2H)-ones and 6-methyl-1,2,3,6,11,11a-hexahydro-4H-pyrido[1,2-b]isoquinolin-4-ones (Compounds of Formula (Ig)), General Procedure D

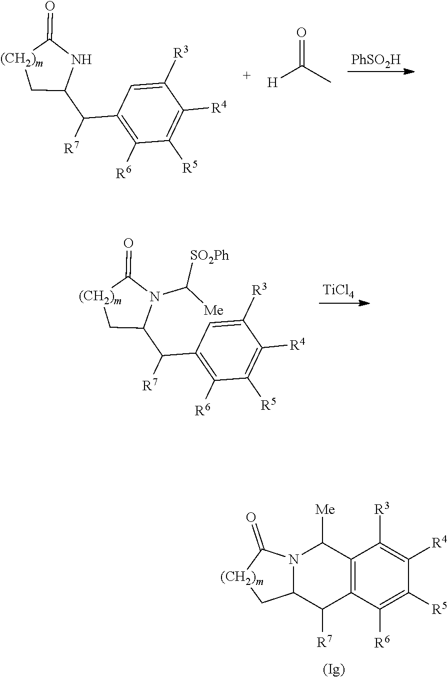

Step 1. A synthesis method from Tetrahedron: Asymmetry 14 (2003) 1171-1178 was adapted; 1.0 mol eq. of optionally substituted 5-benzylpyrrolidin-2-one (m=1) or 6-benzylpiperidin-2-one (m=2) (in the case of protected R⁷—OH groups (e.g. R⁷=silyl ether), 1.0 mol eq. of the respective protected compound (m=1 or 2)) was dissolved in dichloromethane (3 mL/mmol of optionally substituted 5-benzylpyrrolidin-2-one (m=1) or 6-benzylpiperidin-2-one) and then 2.0 mol eq. benzenesulfinic acid, 1.5 mol eq. acetaldehyde and anhydrous MgSO₄ (0.1 g/mmol of optionally substituted 5-benzylpyrrolidin-2-one (m=1) or 6-benzylpiperidin-2-one) were sequentially added at room temperature. The mixture was stirred for 36 h at room temperature and then filtered over a short pad of Florisil. Removal of the solvent afforded the crude sulfone, which was purified by column chromatography (7:3 hexanes-ethyl acetate).

Step 2. 1.0 mol eq. of the above sulfone (2 mmol) was dissolved in CH₂Cl₂ (10 mL/mmol of sulfone), and the solution was cooled at −78° C. 1.5 mol eq. TiCl₄ was then added dropwise in 5 min and after 45 min at −78° C. the reaction mixture was quenched with brine. The aqueous phase obtained after separation was extracted with CH₂Cl₂ three times and the collected organic phase was dried over MgSO₄. After removal of the solvent at reduced pressure the optionally substituted 5-methyl-1,5,10,10a-tetrahydropyrrolo[1,2-b]isoquinolin-3(2H)-one or 6-methyl-1,2,3,6,11,11a-hexahydro-4H-pyrido[1,2-b]isoquinolin-4-one obtained was purified by column chromatography (7:3 hexane-ethyl acetate).

Step 3. In the case of protected R⁷—OH groups, the R⁷—OH protecting groups were removed according to procedures well known in the art, e.g. in the case of 0-trimethylsilyl protected compounds, otherwise skip this step:

To a cold (0° C.) solution of 1.0 mol eq. of the silyl ether in tetrahydrofuran (THF, 10 mL/mol silyl ether) was added 1.1 mol eq. tetra-n-butylammonium fluoride (TBAF) (1 M solution in THF), the resulting solution was stirred for 45 minutes allowing the mixture to warm to room temperature and stirring was continued until conversion was complete. The resulting solution was diluted with DCM and quenched with water. The organic layer was extracted with brine and dried over magnesium sulfate, followed by solvent reduction in vacuo. The crude product was purified by column chromatography (hexane/ethyl acetate, 7:3 to 1:10) to give the alcohol.

Step 4. In the case of R⁷—SH group, otherwise skip this step:

The product from step 3 was converted to the tosylate following step 2 of the general procedure A. The formed tosylate was treated with excess sodium hydrogen sulfide in acetone to give the crude thiol product (R⁷═SH). The crude product was purified by column chromatography (hexane/ethyl acetate, 7:3 to 1:10) to give the thiol.

### General Procedure for the Preparation of Optionally Substituted 5-methyl-1,2,5,6,11,11a-hexahydro-3H-indolizino[6,7-b]indol-3-ones and 6-methyl-6,9,10,11,11a,12-hexahydroindolo[3,2-b]quinolizin-8(5H)-ones (Compounds of Formula (Ie)), General Procedure E

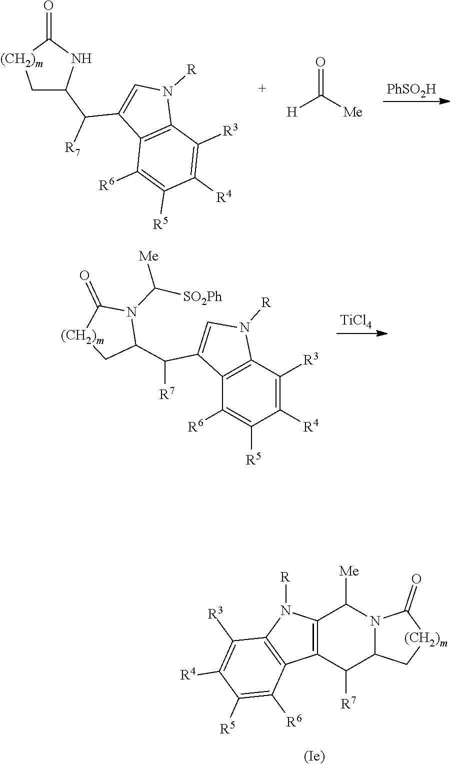

The general procedure D was followed starting from optionally subst. 5-((1H-indol-3-yl)methyl)pyrrolidin-2-one (m=1, R═H, Bn or TIPS) or 6-((1H-indol-3-yl)methyl)piperidin-2-one (m=2, R═H, Bn or TIPS) (in the case of protected R⁷—OH groups (e.g. R⁷=silyl ether), 1.0 mol eq. of the respective protected compound (m=1 or 2)). In the case of protecting groups at R and/or R⁷, the protecting group(s) was/were removed according to procedures well known in the art, e.g. step 3 of general procedure D was followed (in the case R=TIPS and R⁷=silyl ether, 2.2 mol eq. (TBAF) (1 M solution in THF) was used). In the case of R⁷—SH group; general procedure D, step 4, was followed using the deprotected R⁷—OH compound.

### General Procedure for the Preparation of Optionally Substituted 1,5,6,10b-tetrahydropyrrolo[2,1-a]isoquinolin-3(2H)-ones and 1,2,3,6,7,11b-hexahydro-4H-pyrido[2,1-a]isoquinolin-4-ones (Compounds of Formula (If), General Procedure F

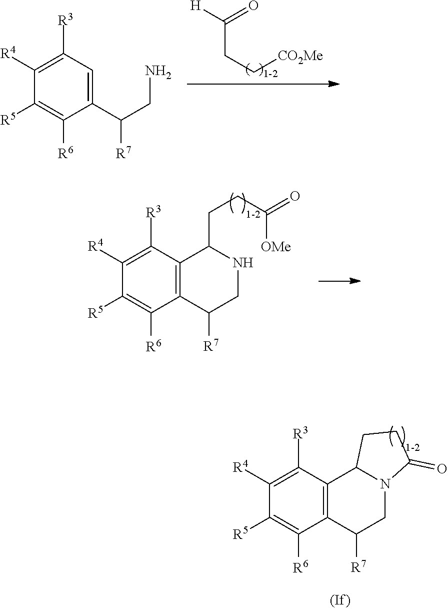

Optionally substituted 1,5,6,10b-tetrahydropyrrolo[2,1-a]isoquinolin-3(2H)-ones and 1,2,3,6,7,11b-hexahydro-4H-pyrido[2,1-a]isoquinolin-4-ones were prepared using methods described in Chem Comm., 2018, 54(11), 1323-1326; shortly, 1.0 mol eq. of optionally substituted phenethylamine from step 5 of general procedure A and 1.5 mol eq. of aldehyde (e.g. methyl 4-oxobutanoate or methyl 5-oxopentanoate) in aqueous potassium phosphate KPi buffer (0.3 M) in acetonitrile (1:1) were stirred under argon for 18 h at 60° ° C. at pH 6 in the presence of 1.0 mol eq. of ascorbic acid, followed by the addition of sodium carbonate (1 M), adjustment of pH to 7.5, and stirring the mixture for 4 h to give the lactams. The lactams were purified by a basic and then acidic extraction procedure using EtOAc and then MeOCO₂Me, as described in Tetrahedron Lett., 2014, 55, 5047 and Nat. Commun., 2017, 8, 14883.

In the case of protected R⁷—OH groups, the R⁷—OH protecting groups were removed according to procedures well known in the art, e.g. in the case of 0-trimethylsilyl protected compounds step 3 of general procedure D was followed. In the case of R⁷—SH group; general procedure D, step 4, was followed using the deprotected R⁷—OH compound.

### General Procedure for the Preparation of Optionally Substituted 1,2,5,6,11,11b-hexahydro-3H-indolizino[8,7-b]indol-3-ones and 2,3,6,7,12,12b-hexahydroindolo[2,3-a]quinolizin-4(1H)-ones (Compounds of Formula (Id), General Procedure G

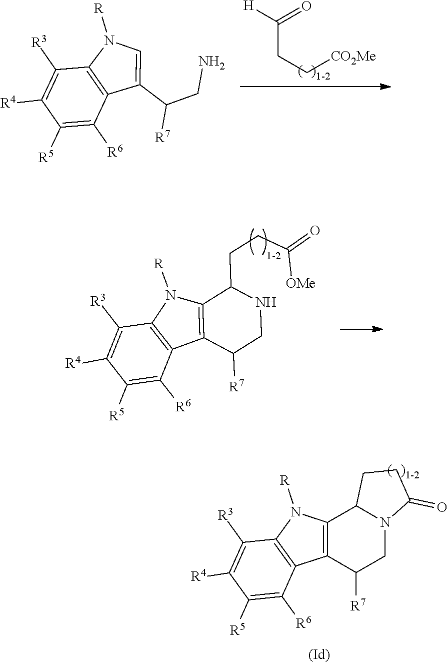

The general procedure F was followed except that optionally subst. 2-(1H-indol-3-yl)ethan-1-amines (R═H) or N-protected 1H-indole-3-carboxaldehydes (e.g. 1-benzyl-1H-indole-3-carboxaldehyde (R=Bn) or 1-(triisopropylsilyl)-1H-indole-3-carbaldehyde (R=TIPS)) from general procedure B were used instead of the optionally subst. phenethylamine. In the case of protecting groups at R and/or R⁷, the protecting group(s) was/were removed according to procedures well known in the art, e.g. step 3 of general procedure D was followed (in the case R=TIPS and R⁷=silyl ether, 2.2 mol eq. (TBAF) (1 M solution in THF) was used). In the case of R⁷—SH group; general procedure D, step 4, was followed using the deprotected R⁷—OH compound.

### General Procedure for the Preparation of Optionally Substituted 1-methyl-2,3,4,9-tetrahydro-1H-pyrido[3,4-b]indoles and 1-methyl-4,9-dihydro-3H-pyrido[3,4-b]indoles (Compounds of Formula (Ic), General Procedure H

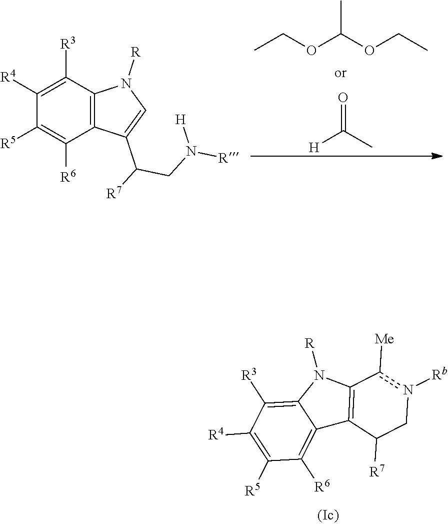

Step 1. Pictet-Spengler reaction—industrial scale (EP 0929527) was adapted: 1.0 mol eq. optionally subst. N-tosyl-2-(1H-indol-3-yl)ethylamine (R═H or protecting group, e.g. Bn or TIPS, R′″=Ts) from general procedure C′ and 3.0 mol eq. boron trifluoride diethyl etherate was refluxed with 21.0 mol eq. of 1,1-diethoxyethane for 12 h in N2 atmosphere or until TLC showed completion. The tosyl was removed using e.g. sodium, naphthalene and dimethoxyethane, or using KOH and MeOH; or

1.0 mol eq. optionally subst. 2-(1H-indol-3-yl)ethan-1-amine from general procedure B was refluxed for 1 h with 3.0 mol eq. acetaldehyde which after 1.2 mol eq. hydrochloric acid 37% was added and refluxing was continued until TLC showed completion.

Step 2. The reaction mixture was partitioned between ethyl acetate (10 mL/1 g substrate) and water (10 mL/1 g substrate), separated, and the organic layer was washed twice with saturated sodium bicarbonate and dried over sodium sulfate (Na₂SO₄). The drying agent was filtered off and the filtrate was distilled under reduced pressure to yield the desired compound. In the case of protecting groups at R and/or R⁷: the protecting groups were removed according to procedures well known in the art, e.g. protecting groups at R and/or R⁷ were removed following e.g. step 3 of general procedure D (in the case R=TIPS and R⁷=silyl ether, 2.2 mol eq. (TBAF) (1 M solution in THF) was used). In the case of R⁷—SH group; general procedure D, step 4, was followed using the deprotected R⁷—OH compound.

### General Procedure for the Preparation of the Compounds of the Invention Using a One-Pot Chemoenzymatic Reaction Cascade Method, the Preparation of Optionally Subst. 1,5,6,10b-tetrahydropyrrolo[2,1-a]isoquinolin-3(2H)-ones as an Example

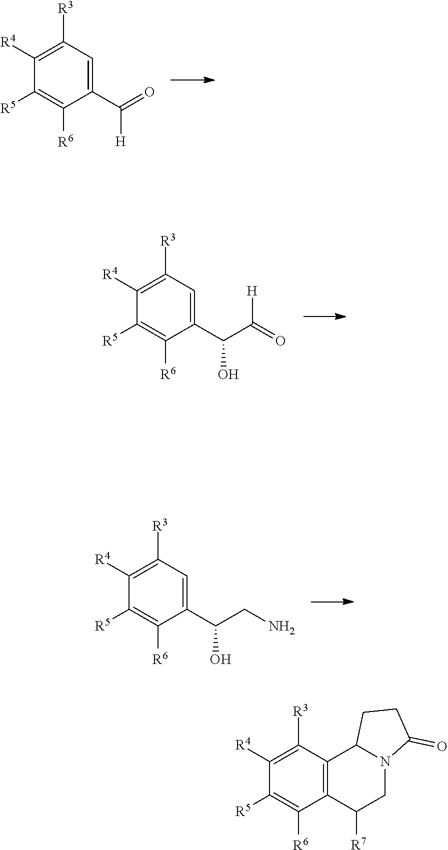

In a one-pot chemoenzymatic synthesis method of the compounds of the invention, acetolactate synthase (ALS) converts an optionally subst. benzaldehyde (e.g. 3,4-difluorobenzaldehyde) to an optionally subst. 2-hydroxy-2-phenylacetaldehyde (e.g. (2R)-(3,4-difluorophenyl)(hydroxyl)acetaldehyde). A transaminase (EC 2.6.1) then converts the formed optionally subst. (R)-2-hydroxy-2-phenylacetaldehyde to the respective optionally subst. phenethylamine, which is then converted by a norcoclaurine synthase ((S)-norcoclaurine synthase (EC 4.2.1.78)) to an optionally subst. 1,5,6,10b-tetrahydropyrrolo[2,1-a]isoquinolin-3(2H)-one (e.g. 8,9-difluoro-6-hydroxy-1,5,6,10b-tetrahydropyrrolo[2,1-a]isoquinolin-3(2H)-one). Optionally, the products may be purified according to procedures well known in the art. Optionally, the R⁷—OH may be converted to a halogen, OH, oxo, SH, NOR⁸, C₁₋₃-(per)haloalkyl, C₁₋₃-alkoxy, C₁₋₃-(per)haloalkoxy, CN, C(O)N(R⁸)₂, or N(R⁸)₂ by synthesis methods described herein or conventional processes well-known to the person skilled in the art.

The one-pot chemoenzymatic reaction cascade method can also be used to synthesize other compounds with formula (I), (Ia), (Ib), (Ic), (Id), (Ie), (If), and (Ig) of the invention. Therefore, e.g. an optionally subst. 1H-indole-3-carbaldehyde or an optionally subst. N-protected 1H-indole-3-carboxaldehyde (e.g. 1-benzyl-1H-indole-3-carboxaldehyde or 1-(triisopropylsilyl)-1H-indole-3-carbaldehyde) may be used as a starting material in the one-pot chemoenzymatic synthesis method to give optionally subst. 1,2,5,6,11,11b-hexahydro-3H-indolizino[8,7-b]indol-3-ones and 2,3,6,7,12,12b-hexahydroindolo[2,3-a]quinolizin-4(1H)-ones:

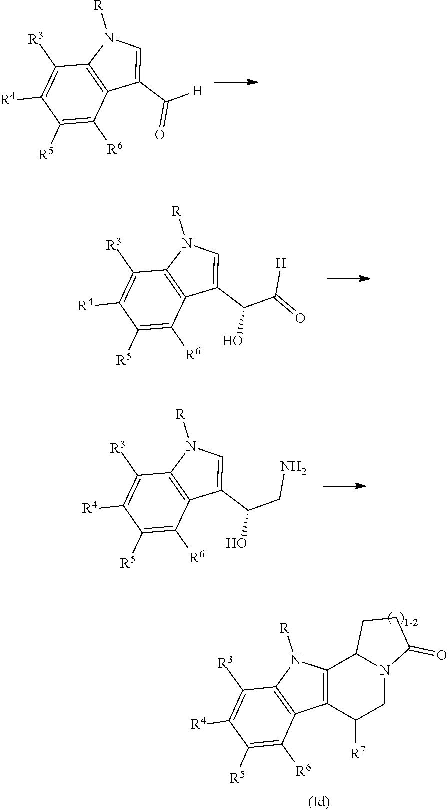

The one-pot chemoenzymatic synthesis method may also comprise other enzymes, depending on the compound to be synthesized. Also, one or more intermediate products (e.g. an optionally subst. phenethylamine or an optionally subst. 2-(1H-indol-3-yl)ethan-1-amine) may be used in the one-pot chemoenzymatic synthesis method or the method may be used on a reaction product of one or more of the above disclosed chemical methods for the preparation of compounds.

Alternatively, or additionally, chemical reactions may be performed on any of the above-mentioned products. E.g., the optionally subst. 2-hydroxy-2-phenylacetaldehyde produced by an enzymatic reaction may be isolated, optionally purified, and may be converted to the 2-fluoro-2-phenylacetaldehyde as described in general procedure A. The formed 2-fluoro-2-phenylacetaldehyde may thereafter be subjected to a one-pot chemoenzymatic synthesis method to give 6-fluoro-1,5,6,10b-tetrahydropyrrolo[2,1-a]isoquinolin-3(2H)-one.

Additionally, or alternatively, protecting groups may be inserted by chemical reactions on products produced, optionally isolated, and thereafter subjected to a one-pot chemoenzymatic synthesis method. Additionally, the compounds may be deprotected from protecting groups after the final enzymatic reaction, or alternatively before, at or after any step of the enzymatic reactions.

One or more expression plasmid comprising genes encoding for the required enzymatic activities in functional linkage with the required regulatory sequences may be used in the one-pot chemoenzymatic synthesis method. A single plasmid comprising all genes for the required enzymatic activities represents a particular convenient embodiment. A plasmid suitable for use in such a method is exemplified in SEQ ID NO: 1 shown in the sequence protocol, which forms part of the present disclosure.

As illustrated by SEQ ID NO: 1, said one or more plasmid encodes for
acetolactate synthase activity (e.g., AHAS 1, further exemplified by nucleotides 1142 to 3097 of SEQ ID NO: 1),
for an enzyme with EC 2.6.1 activity (e.g. a suitable transaminase, for example transaminase E1V913_HALED, in particular as further exemplified in nucleotides 4603 to 5985 of SEQ ID NO: 1),
for an enzyme with salsolinol synthase activity (in particular as further exemplified in nucleotides 6779 to 7009 of SEQ ID NO: 1),
for an enzyme with EC 4.2.1.78 activity (e.g. a suitable norcolaurine synthase, such as the norcolaurine synthase as exemplified in nucleotides 10117 to 10689 of SEQ ID NO: 1), and
for an enzyme with EC 2.1.1.28 activity (e.g. a suitable phenylethanolamine N-methyltransferase (PNMT), such as the P11086 PNMT, as further exemplified in nucleotides 14784 to 15335 of SEQ ID NO: 1.

The regulatory sequences comprise sequences required to bring about the expression, translation and secretion of the enzymes, such as enhancers, promoters, sequences encoding for signal peptides (such as for the AmyE signal peptide, sortase cleavage signal, or PrsA), terminators, 5′ untranslated regions (5′ UTR), 3′ untranslated regions (3′ UTR), and transcription regulatory sequences (e.g. of the arabinose operon or the CUP operon). Usually, the plasmid will also comprise additional sequences required for maintenance of the plasmid, for example a replication origin (e.g., oriU) and/or a reporter or selection gene (such as the mRaspberry reporter). Examples of such sequences are shown in SEQ ID NO: 1. However, the skilled person is aware of alternatives forming part of the skilled person's common general knowledge, the sequences of which are publicly available in sequence databases.

Any suitable host which is compatible with the selected sequences and codon usage of the plasmid may be used. For example, for the plasmid exemplified in SEQ ID NO: 1, a suitable Bacillus strain, such as a suitable Bacillus subtilis strain, may be used as the host. For example, Bacillus subtilis (WB-600) may be used as the electrocompetent host. Methods for introducing the plasmid into the host cell are generally known to the skilled person and are provided in reference text-books. The host cell comprising the plasmid, such that the enzymatic activities are expressed and secreted in the environment of the host cell, may then be used in the one-pot chemoenzymatic synthesis method. The host cell may be used directly in the synthesis method, or only the supernatant of a fermentation of said host cell. Suitable fermentation procedures are also known to the skilled person, and publicly available, for example from catalogues of cell culture collections, and depend on the selected host cell.

The pharmaceutically acceptable salts of the compounds of formula (I) may be prepared by conventional processes well-known to the person skilled in the art. For the preparation of pharmaceutical compositions and dosage forms as well as the carriers, diluents and expedients used in the preparation, see, for example, Remington's Pharmaceutical Sciences, 20th Edition, 2000, Marck Publishing Company, Easton, Pennsylvania.

The pharmacological activity of the compounds of the invention can be verified by methods known in the art. For example, the reducing effect on alcohol seeking behavior can be verified using the procedure described by Heidbreder, C. A., et al., Addict Biol. 2007 March; 12(1):35-50. The parkinsonism-preventing activity can be shown, for example, as described by Okuda, K., et al. Biol Pharm Bull. 2006 July; 29(7):1401-1403.

The following specific non-limiting examples will further identify the compounds of the invention.

### Compound 1

### 5-Fluoro-1-methyl-2,3,4,9-tetrahydro-1H-pyrido[3,4-b]indole

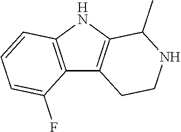

The general procedure B was used starting from 4-fluoro-1H-indole-3-carbaldehyde, followed by general procedures C′ and H.

¹H NMR: δ 1.21 (3H, d, J=6.8 Hz), 2.89 (1H, ddd, J=10.1, 7.4, 3.9 Hz), 2.91 (1H, ddd, J=14.4, 3.9, 2.0 Hz), 2.76 (1H, ddd, J=14.4, 10.1, 3.7 Hz)), 3.01 (1H, ddd, J=7.4, 3.7, 2.0 Hz), 4.11 (1H, q, J=6.8 Hz), 6.82 (1H, dd, J=7.7, 1.8 Hz), 6.78 (1H, dd, J=7.5, 1.8 Hz)), 7.01 (1H, dd, J=7.7, 7.5 Hz).

### Compound 2

### 6-Fluoro-1-methyl-2,3,4,9-tetrahydro-1H-pyrido[3,4-b]indole

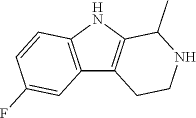

The general procedure B was used starting from 5-fluoro-1H-indole-3-carbaldehyde, followed by general procedures C′ and H.

¹H NMR: δ 1.21 (3H, d, J=6.8 Hz), 2.75 (1H, ddd, J=12.9, 10.1, 3.7 Hz), 3.00 (1H, ddd, J=7.4, 3.7, 2.0 Hz), 2.99 (1H, ddd, J=12.9, 3.9, 2.0 Hz), 2.89 (1H, ddd, J=10.1, 7.4, 3.9 Hz), 4.14 (1H, q, J=6.8 Hz), 6.86 (1H, dd, J=8.5, 0.4 Hz), 7.03 (1H, dd, J=8.5, 1.1 Hz), 7.01 (1H, dd, J=1.1, 0.4 Hz)).

### Compound 3

### 1-Methyl-4,9-dihydro-3H-pyrido[3,4-b]indole

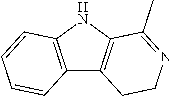

The general procedure B was used starting from 1H-indole-3-carbaldehyde, followed by general procedures C′ and H.

¹H NMR: δ 2.09 (3H, s), 3.09 (2H, ddd, J=14.8, 4.7, 1.7 Hz), 3.76 (2H, ddd, J=13.1, 9.7, 4.7 Hz), 6.96 (1H, ddd, J=8.3, 7.6, 1.3 Hz), 7.13 (1H, ddd, J=8.0, 1.3, 0.5 Hz), 7.19 (1H, ddd, J=8.0, 7.6, 1.3 Hz), 7.57 (1H, ddd, J=8.3, 1.3, 0.5 Hz).

### Compound 4

### 4,6-Difluoro-1-methyl-2,3,4,9-tetrahydro-1H-pyrido[3,4-b]indole

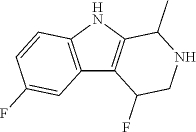 [SMILES: CC1NCC(F)c2c1[nH]c1ccc(F)cc21]

The general procedure B was used starting from 5-fluoro-1H-indole-3-carbaldehyde, followed by general procedures C′ and H.

¹H NMR: 1.60 (s, 2H), 3.36 (dddd, J=4.6, 5.3, 13.9, 17.2 Hz, 1H), 3.55 (dddd, J=1.8, 5.3, 13.7, 17.2 Hz, 1H), 4.01 (dtd, J=3.3, 5.3, 6.2 Hz), 4.48 (dq, J=3.8, 6.4 Hz, 1H), 6.75 (dd, J=1.8, 4.6 Hz, 1H), 6.85 (dd, J=1.8, 4.6 Hz, 1H), 7.16 (ddd, J=2.7, 7.1, 9.9 Hz, 1H), 7.24 (dd, J=2.6, 12.1 Hz, 1H), 7.28-7.34 (m, 1H), 8.36 (s, 1H)

### Compound 5

### 7-Fluoro-1H,2H,3H,4H,6H,7H,12H,12bH-indolo[2,3-a]quinolizin-4-one

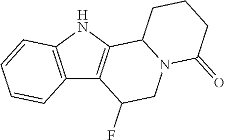 [SMILES: O=C1CCCC2c3[nH]c4ccccc4c3C(F)CN12]

The general procedure B was used starting from 1H-indole-3-carbaldehyde, followed by general procedure G.

¹H NMR: δ 1.86 (1H, ddddd, J=13.0, 3.5, 3.2, 2.5, 2.2 Hz), 1.86 (1H, ddddd, J=13.0, 10.2, 10.2, 3.5, 2.4 Hz)), 2.05 (1H, dddd, J=13.5, 4.9, 3.2, 2.4 Hz), 2.09 (1H, dddd, J=13.5, 10.2, 9.4, 2.5 Hz)), 2.33 (1H, ddd, J=14.6, 3.5, 2.2 Hz), 2.29 (1H, ddd, J=14.6, 10.2, 3.5 Hz), 4.14 (1H, dd, J=12.3, 1.7 Hz), 4.10 (1H, dd, J=12.3, 4.4 Hz), 5.22 (1H, dd, J=9.4, 4.9 Hz), 5.92 (1H, dd, J=4.4, 1.7 Hz), 6.94 (1H, ddd, J=8.0, 7.6, 1.2 Hz), 7.10 (1H, ddd, J=7.8, 1.2, 0.6 Hz), 7.12 (1H, ddd, J=7.8, 7.6, 1.1 Hz)), 7.44 (1H, ddd, J=8.0, 1.1, 0.6 Hz).

### Compound 6

### 10-Fluoro-1H,2H,3H,4H,6H, 7H,12H,12bH-indolo[2,3-a]quinolizin-4-one

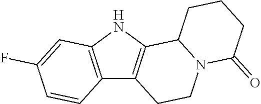 [SMILES: O=C1CCCC2c3[nH]c4cc(F)ccc4c3CCN12]

The general procedure B was used starting from 6-fluoro-1H-indole-3-carbaldehyde, followed by general procedure G.

¹H NMR: δ 2.10 (1H, dddd, J=13.4, 10.2, 9.4, 2.5 Hz), 1.98 (1H, dddd, J=13.4, 4.8, 3.2, 2.4 Hz), 1.86 (1H, ddddd, J=13.0, 3.5, 3.2, 2.5, 2.2 Hz), 1.86 (1H, ddddd, J=13.0, 10.2, 10.2, 3.4, 2.4 Hz), 2.31 (1H, ddd, J=14.3, 10.2, 3.5 Hz), 2.33 (1H, ddd, J=14.3, 3.4, 2.2 Hz)), 2.88 (1H, ddd, J=12.9, 9.8, 4.4 Hz), 2.95 (1H, ddd, J=12.9, 4.4, 1.6 Hz), 3.62 (1H, ddd, J=13.1, 9.8, 4.4 Hz), 3.81 (1H, ddd, J=13.1, 4.4, 1.6 Hz), 5.28 (1H, dd, J=9.4, 4.8 Hz), 6.77 (1H, dd, J=1.9, 0.5 Hz), 6.79 (1H, dd, J=7.7, 1.9 Hz)), 7.35 (1H, dd, J=7.7, 0.5 Hz).

### Compound 7

### 7,8,9,10-Tetrafluoro-1H,2H,3H,4H,6H,7H,12H,12bH-indolo[2,3-a]quinolizin-4-one

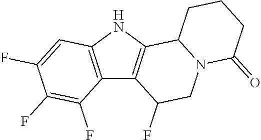 [SMILES: O=C1CCCC2c3[nH]c4cc(F)c(F)c(F)c4c3C(F)CN12]

The general procedure B was used starting from 4,5,6-trifluoro-1H-indole-3-carbaldehyde, followed by general procedure G.

¹H NMR δ 1.73-1.85 (m, 1H), 2.06 (ddt, J=5.5, 8.1, 12.0 Hz, 1H), 2.21-2.44 (m, 3H), 2.49 (ddd, J=5.3, 7.7, 14.2 Hz, 1H), 4.02 (ddd, J=5.3, 12.7, 17.0 Hz, 1H), 4.23 (ddd, J=2.6, 12.8, 17.0 Hz, 1H), 5.08 (dd, J=2.6, 5.4 Hz, 1H), 6.75-6.82 (m, 2H), 6.88 (dd, J=2.6, 5.3 Hz, 1H), 9.03-9.06 (s, 1H).

### Compound 8

### 7,9-Difluoro-1H,2H,3H,4H,6H,7H,12H,12bH-indolo[2,3-a]quinolizin-4-one

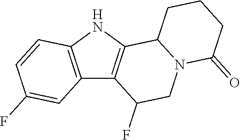 [SMILES: O=C1CCCC2c3[nH]c4ccc(F)cc4c3C(F)CN12]

The general procedure B was used starting from 5-fluoro-1H-indole-3-carbaldehyde, followed by general procedure G.

¹H NMR: δ 1.87 (1H, ddddd, J=13.0, 3.5, 3.2, 2.5, 2.2 Hz), 1.86 (1H, ddddd, J=13.0, 10.2, 10.2, 3.5, 2.4 Hz)), 2.05 (1H, dddd, J=13.4, 4.9, 3.2, 2.4 Hz), 2.03 (1H, dddd, J=13.4, 10.2, 9.4, 2.5 Hz), 2.33 (1H, ddd, J=14.6, 3.5, 2.2 Hz), 2.29 (1H, ddd, J=14.6, 10.2, 3.5 Hz), 3.98 (1H, dd, J=12.2, 4.4 Hz), 4.11 (1H, dd, J=12.2, 1.7 Hz), 5.24 (1H, dd, J=9.4, 4.9 Hz), 5.98 (1H, dd, J=4.4, 1.7 Hz), 6.93 (1H, dd, J=8.5, 1.1 Hz), 7.12 (1H, dd, J=8.5, 0.5 Hz), 7.32 (1H, dd, J=1.1, 0.5 Hz).

### Compound 9

### 7,8,10-Trifluoro-1H,2H,3H,4H,6H,7H,12H,12bH-indolo[2,3-a]quinolizin-4-one

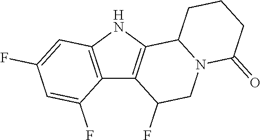 [SMILES: O=C1CCCC2c3[nH]c4cc(F)cc(F)c4c3C(F)CN12]

The general procedure B was used starting from 4,6-difluoro-1H-indole-3-carbaldehyde, followed by general procedure G.

¹H NMR: δ 2.00 (1H, dddd, J=13.2, 4.9, 3.2, 2.4 Hz), 1.85 (1H, ddddd, J=13.0, 10.2, 10.2, 3.5, 2.4 Hz), 1.86 (1H, ddddd, J=13.0, 3.5, 3.2, 2.5, 2.2 Hz), 2.00 (1H, dddd, J=13.2, 10.2, 9.4, 2.5 Hz)), 2.33 (1H, ddd, J=14.6, 3.5, 2.2 Hz), 2.29 (1H, ddd, J=14.6, 10.2, 3.5 Hz)), 3.93 (1H, dd, J=12.1, 4.4 Hz), 4.08 (1H, dd, J=12.1, 1.7 Hz), 5.21 (1H, dd, J=9.4, 4.9 Hz), 6.02 (1H, dd, J=4.4, 1.7 Hz), 6.79 (1H, d, J=2.2 Hz), 6.88 (1H, d, J=2.2 Hz).

### Compound 10

### 4,5,6,7-Tetrafluoro-1-methyl-2,3,4,9-tetrahydro-1H-pyrido[3,4-b]indole

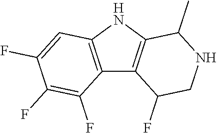 [SMILES: CC1NCC(F)c2c1[nH]c1cc(F)c(F)c(F)c21]

The general procedure B was used starting from 4,5,6-trifluoro-1H-indole-3-carbaldehyde, followed by general procedures C′ and H.

¹H NMR: δ 1.25 (3H, d, J=6.9 Hz), 3.06 (1H, dd, J=13.7, 3.9 Hz), 3.16 (1H, dd, J=13.7, 2.0 Hz), 4.24 (1H, q, J=6.9 Hz), 5.88 (1H, dd, J=3.9, 2.0 Hz), 7.15 (1H, s).

### Compound 11

### 5,6-Difluoro-1-methyl-4,9-dihydro-3H-pyrido[3,4-b]indole

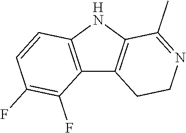 [SMILES: CC1=NCCc2c1[nH]c1ccc(F)c(F)c21]

The general procedure B was used starting from 5,6-difluoro-1H-indole-3-carbaldehyde, followed by general procedures C′ and H.

¹H NMR δ 2.51 (s, 3H), 4.06 (dd, J=5.4, 6.3 Hz, 2H), 4.22 (dd, J=5.4, 6.3 Hz, 2H), 7.12-7.23 (m, 2H), 8.94 (s, 1H).

### Compound 12

### 5,6,7-Trifluoro-1-methyl-4,9-dihydro-3H-pyrido[3,4-b]indole

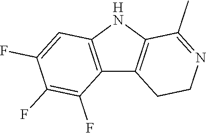 [SMILES: CC1=NCCc2c1[nH]c1cc(F)c(F)c(F)c21]

The general procedure B was used starting from 4,5,6-trifluoro-1H-indole-3-carbaldehyde, followed by general procedures C′ and H.

¹H NMR: δ 2.08 (3H, s), 3.02 (2H, ddd, J=13.3, 4.7, 1.7 Hz), 3.45 (2H, ddd, J=12.9, 9.7, 4.7 Hz), 7.17 (1H, s).

### Compound 13

### 4,5,6,7-Tetrafluoro-1-methyl-4,9-dihydro-3H-pyrido[3,4-b]indole

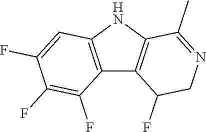 [SMILES: CC1=NCC(F)c2c1[nH]c1cc(F)c(F)c(F)c21]

The general procedure B was used starting from 4,5,6-trifluoro-1H-indole-3-carbaldehyde, followed by general procedures C′ and H.

¹H NMR: δ 2.10 (3H, s), 3.82 (1H, dd, J=13.7, 1.7 Hz), 3.83 (1H, dd, J=13.7, 4.6 Hz), 5.97 (1H, dd, J=4.6, 1.7 Hz), 7.08 (1H, s).

### Compound 14

### 7,8,10-Trifluoro-5-methyl-1,5,10,10a-tetrahydropyrrolo[1,2-b]isoquinolin-3(2H)-one

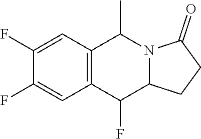 [SMILES: CC1c2cc(F)c(F)cc2C(F)C2CCC(=O)N12]

The general procedure D was used starting from 5-((3,4-difluorophenyl)fluoromethyl)pyrrolidin-2-one.

¹H NMR δ 1.35 (s, 3H), 1.60-1.73 (m, 4H), 2.41-2.50 (dddd, J=1.8, 6.4, 7.3, 13.9 Hz, 2H), 2.58-2.67 (ddd, J=6.4, 7.5, 13.8 Hz, 2H), 4.27-4.36 (m, 2H), 5.00-5.08 (dtd, J=3.8, 6.3, 6.3, 7.0 Hz, 2H), 5.53-5.57 (dd, J=0.7, 3.3 Hz, 1H), 5.64-5.68 (d, J=3.3 Hz, 1H), 7.04-7.11 (m, 2H), 7.16-7.24 (m, 2H).

### Compound 15

### 7,10-Difluoro-5-methyl-1,5,10,10a-tetrahydropyrrolo[1,2-b]isoquinolin-3(2H)-one

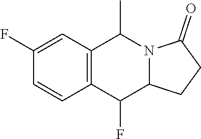 [SMILES: CC1c2cc(F)ccc2C(F)C2CCC(=O)N12]

The general procedure D was used starting from 5-(fluoro(4-fluorophenyl)methyl)pyrrolidin-2-one.

¹H NMR: δ 1.35 (3H, d, J=6.9 Hz), 2.13 (1H, dddd, J=14.0, 8.1, 4.2, 4.2 Hz), 2.02 (1H, dtd, J=14.0, 8.1, 4.1 Hz)), 2.41 (1H, ddd, J=16.8, 8.1, 4.2 Hz), 2.45 (1H, ddd, J=16.8, 8.1, 4.1 Hz), 4.00 (1H, ddd, J=8.1, 4.5, 4.2 Hz), 5.00 (1H, q, J=6.9 Hz), 5.74 (1H, d, J=4.5 Hz), 6.93 (1H, dd, J=8.5, 1.4 Hz), 7.17 (1H, dd, J=8.5, 0.5 Hz), 7.15 (1H, dd, J=1.4, 0.5 Hz)).

### Compound 16

### 9,10-Difluoro-5-methyl-1,5,10,10a-tetrahydropyrrolo[1,2-b]isoquinolin-3(2H)-one

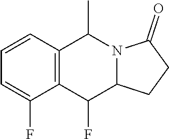 [SMILES: CC1c2cccc(F)c2C(F)C2CCC(=O)N12]

The general procedure D was used starting from 2 5-(fluoro(2-fluorophenyl)methyl)pyrrolidin-2-one.

¹H NMR: δ 1.35 (3H, d, J=6.9 Hz), 2.13 (1H, dddd, J=13.9, 8.1, 4.2, 4.2 Hz), 2.03 (1H, dtd, J=13.9, 8.1, 4.1 Hz)), 2.41 (1H, ddd, J=16.8, 8.1, 4.2 Hz), 2.45 (1H, ddd, J=16.8, 8.1, 4.1 Hz)), 4.09 (1H, ddd, J=8.1, 4.5, 4.2 Hz), 5.06 (1H, q, J=6.9 Hz), 5.91 (1H, d, J=4.5 Hz), 6.93 (1H, dd, J=8.3, 1.0 Hz), 6.97 (1H, dd, J=8.3, 7.7 Hz)), 7.05 (1H, dd, J=7.7, 1.0 Hz).

### Compound 17

### 7,8-Difluoro-5-methyl-1,5,10,10a-tetrahydropyrrolo[1,2-b]isoquinolin-3(2H)-one

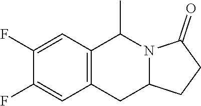 [SMILES: CC1c2cc(F)c(F)cc2CC2CCC(=O)N21]

The general procedure D was used starting from 5-(3,4-difluorobenzyl)pyrrolidin-2-one.

¹H NMR: δ 1.29 (3H, d, J=6.9 Hz), 2.06 (1H, dddd, J=13.7, 8.1, 4.2, 4.2 Hz), 2.09 (1H, dtd, J=13.7, 8.1, 4.1 Hz)), 2.38 (1H, ddd, J=16.8, 8.1, 4.2 Hz), 2.40 (1H, ddd, J=16.8, 8.1, 4.1 Hz)), 2.69 (1H, dd, J=14.8, 9.6 Hz), 2.88 (1H, dd, J=14.8, 4.5 Hz), 3.80 (1H, dddd, J=9.6, 8.1, 4.5, 4.2 Hz), 5.06 (1H, q, J=6.9 Hz), 7.12 (1H, d, J=0.5 Hz), 7.43 (1H, d, J=0.5 Hz).

### Compound 18

### (5R,11S)-10,11-Difluoro-5-methyl-1,2,5,6,11,11a-hexahydro-3H-indolizino[6,7-b]indol-3-one

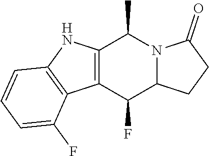 [SMILES: C[C@@H]1c2[nH]c3cccc(F)c3c2[C@H](F)C2CCC(=O)N21]

The general procedure E was followed starting from 5-((S)-fluoro(4-fluoro-1-(triisopropylsilyl)-1H-indol-3-yl)methyl)pyrrolidin-2-one or 5-((S)-(1-benzyl-4-fluoro-1H-indol-3-yl)fluoromethyl)pyrrolidin-2-one.

¹H NMR: δ 1.42 (3H, d, J=6.9 Hz), 2.05 (1H, dddd, J=14.1, 8.1, 4.2, 4.1 Hz), 2.07 (1H, dtd, J=14.1, 8.1, 4.2 Hz)), 2.40 (1H, ddd, J=16.9, 8.1, 4.2 Hz), 2.43 (1H, ddd, J=16.9, 8.1, 4.2 Hz)), 4.27 (1H, ddd, J=9.4, 8.1, 4.1 Hz), 5.58 (1H, q, J=6.9 Hz), 5.59 (1H, d, J=9.4 Hz)), 6.88 (1H, dd, J=7.5, 1.7 Hz), 7.03 (1H, dd, J=7.7, 7.5 Hz), 6.96 (1H, dd, J=7.7, 1.7 Hz)).

### Compound 19

### (6R,12S)-12-Fluoro-6-methyl-6,9,10,11,11a,12-hexahydroindolo[3,2-b]quinolizin-8(5H)-one

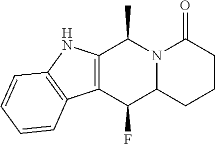 [SMILES: C[C@@H]1c2[nH]c3ccccc3c2[C@H](F)C2CCCC(=O)N21]

The general procedure E was followed starting from 6-((S)-fluoro(1-(triisopropylsilyl)-1H-indol-3-yl)methyl)piperidin-2-one or 6-((S)-(1-benzyl-1H-indol-3-yl)fluoromethyl)piperidin-2-one.

¹H NMR: δ 1.46 (3H, d, J=6.9 Hz), 1.74 (1H, dddd, J=14.2, 10.3, 8.0, 2.6 Hz), 1.84 (1H, dtdd, J=13.1, 10.3, 3.0, 2.5 Hz), 1.73 (1H, dddd, J=14.2, 6.3, 3.1, 2.5 Hz), 1.94 (1H, ddddd, J=13.1, 3.1, 2.9, 2.6, 2.6 Hz), 2.33 (1H, ddd, J=14.7, 3.0, 2.6 Hz), 2.32 (1H, ddd, J=14.7, 10.3, 2.9 Hz), 4.29 (1H, ddd, J=9.9, 8.0, 6.3 Hz), 5.32 (1H, q, J=6.9 Hz), 5.90 (1H, d, J=9.9 Hz), 6.94 (1H, ddd, J=8.0, 7.6, 1.2 Hz), 7.10 (1H, ddd, J=7.8, 1.2, 0.6 Hz), 7.12 (1H, ddd, J=7.8, 7.6, 1.3 Hz)), 7.44 (1H, ddd, J=8.0, 1.3, 0.6 Hz).

### Compound 20

### (5R,11S)-11-Fluoro-5-methyl-1,2,5,6,11,11a-hexahydro-3H-indolizino[6,7-b]indol-3-one

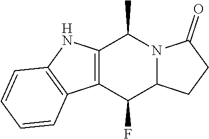 [SMILES: C[C@@H]1c2[nH]c3ccccc3c2[C@H](F)C2CCC(=O)N21]

The general procedure E was followed starting from 5-((S)-fluoro(1-(triisopropylsilyl)-1H-indol-3-yl)methyl)pyrrolidin-2-one or 5-((S)-(1-benzyl-1H-indol-3-yl)fluoromethyl)pyrrolidin-2-one.

¹H NMR: δ 1.46 (3H, d, J=6.9 Hz), 1.74 (1H, dddd, J=14.2, 10.3, 8.0, 2.6 Hz), 1.84 (1H, dtdd, J=13.1, 10.3, 3.0, 2.5 Hz), 1.73 (1H, dddd, J=14.2, 6.3, 3.1, 2.5 Hz), 1.94 (1H, ddddd, J=13.1, 3.1, 2.9, 2.6, 2.6 Hz), 2.33 (1H, ddd, J=14.7, 3.0, 2.6 Hz), 2.32 (1H, ddd, J=14.7, 10.3, 2.9 Hz)), 4.29 (1H, ddd, J=9.9, 8.0, 6.3 Hz), 5.32 (1H, q, J=6.9 Hz), 5.90 (1H, d, J=9.9 Hz), 6.94 (1H, ddd, J=8.0, 7.6, 1.2 Hz), 7.10 (1H, ddd, J=7.8, 1.2, 0.6 Hz), 7.12 (1H, ddd, J=7.8, 7.6, 1.3 Hz), 7.44 (1H, ddd, J=8.0, 1.3, 0.6 Hz).

### Compound 21

### (6R,12S)-1,12-Difluoro-6-methyl-6,9,10,11,11a,12-hexahydroindolo[3,2-b]quinolizin-8(5H)-one

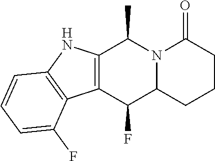 [SMILES: C[C@@H]1c2[nH]c3cccc(F)c3c2[C@H](F)C2CCCC(=O)N21]

The general procedure E was followed starting from (S)-6-((S)-fluoro(4-fluoro-1-(triisopropylsilyl)-1H-indol-3-yl)methyl)piperidin-2-one or 6-((S)-(1-benzyl-4-fluoro-1H-indol-3-yl)fluoromethyl)piperidin-2-one.

¹H NMR: δ 1.42 (3H, d, J=7.0 Hz), 1.74 (1H, dddd, J=14.2, 10.3, 8.0, 2.6 Hz), 1.84 (1H, dtdd, J=13.1, 10.3, 3.0, 2.5 Hz), 1.73 (1H, dddd, J=14.2, 6.3, 3.1, 2.5 Hz), 1.94 (1H, ddddd, J=13.1, 3.1, 2.9, 2.6, 2.6 Hz), 2.33 (1H, ddd, J=14.7, 3.0, 2.6 Hz), 2.32 (1H, ddd, J=14.7, 10.3, 2.9 Hz), 4.27 (1H, ddd, J=9.9, 8.0, 6.3 Hz), 5.31 (1H, q, J=7.0 Hz), 5.88 (1H, d, J=9.9 Hz), 6.88 (1H, dd, J=7.5, 1.7 Hz), 7.03 (1H, dd, J=7.7, 7.5 Hz), 6.97 (1H, dd, J=7.7, 1.7 Hz)).

### Compound 22

### (5R,11aS)-9-Fluoro-5-methyl-1,2,5,6,11,11a-hexahydro-3H-indolizino[6,7-b]indol-3-one

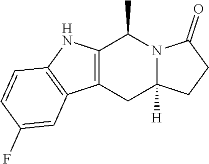 [SMILES: C[C@@H]1c2[nH]c3ccc(F)cc3c2C[C@@H]2CCC(=O)N21]

The general procedure E was followed starting from 5-((5-fluoro-1-(triisopropylsilyl)-1H-indol-3-yl) methyl)pyrrolidin-2-one or 5-((1-benzyl-6-fluoro-1H-indol-3-yl)methyl)pyrrolidin-2-one.

¹H NMR: δ 1.43 (3H, d, J=6.9 Hz), 2.05 (1H, dddd, J=13.8, 8.1, 4.2, 4.1 Hz), 2.13 (1H, dtd, J=13.8, 8.1, 4.2 Hz)), 2.38 (1H, ddd, J=16.8, 8.1, 4.2 Hz), 2.43 (1H, ddd, J=16.8, 8.1, 4.2 Hz), 2.81 (1H, dd, J=14.5, 9.4 Hz), 2.85 (1H, dd, J=14.5, 5.0 Hz), 4.27 (1H, dddd, J=9.4, 8.1, 5.0, 4.1 Hz), 5.59 (1H, q, J=6.9 Hz), 6.92 (1H, dd, J=8.5, 1.1 Hz), 6.86 (1H, dd, J=8.5, 0.5 Hz), 7.24 (1H, dd, J=1.1, 0.5 Hz).

### Compound 23

### (6R,11aS)-2-Fluoro-6-methyl-6,9,10,11,11a,12-hexahydroindolo[3,2-b]quinolizin-8(5H)-one

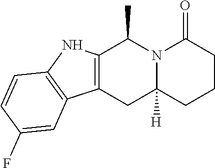 [SMILES: C[C@@H]1c2[nH]c3ccc(F)cc3c2C[C@@H]2CCCC(=O)N21]

The general procedure E was followed starting from 6-((5-fluoro-1-(triisopropylsilyl)-1H-indol-3-yl) methyl)piperidin-2-one or 6-((1-benzyl-5-fluoro-1H-indol-3-yl)methyl)piperidin-2-one.

¹H NMR δ 1.39-1.43 (s, 3H), 3.50-3.62 (m, 2H), 4.15-4.23 (ddd, J=0.8, 7.0, 13.9 Hz, 1H), 4.41-4.49 (ddd, J=0.8, 4.4, 13.9 Hz, 1H), 4.66-4.74 (dtdd, J=1.3, 2.4, 4.6, 4.6, 7.1 Hz, 1H), 5.18-5.25 (qd, J=2.4, 5.5, 5.5, 5.5 Hz, 1H), 5.84-5.95 (m, 2H), 7.12-7.20 (m, 2H), 7.22-7.29 (ddd, J=1.0, 4.8, 6.6 Hz, 1H), 8.61-8.64 (s, 1H).

### Compound 24

### (4S)-4,6-difluoro-1-methyl-4,9-dihydro-3H-pyrido[3,4-b]indole

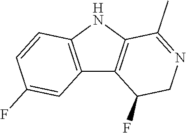 [SMILES: CC1=NC[C@@H](F)c2c1[nH]c1ccc(F)cc21]

The general procedure H was used starting from (2S)-2-fluoro-2-(5-fluoro-1H-indol-3-yl)ethanamine.

¹H NMR δ 3.87-4.07 (m, 3H), 6.98-7.08 (m, 2H), 7.16 (dd, J=3.4, 6.1 Hz, 1H), 7.37-7.44 (m, 3H), 9.04 (s, 1H).

### Compound 25

### (5S,6S,10bS)-6,9-difluoro-5-methyl-1,5,6,10b-tetrahydropyrrolo[2,1-a]isoquinolin-3(2H)-one

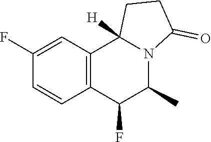 [SMILES: C[C@H]1[C@@H](F)c2ccc(F)cc2[C@@H]2CCC(=O)N21]

The general procedure A was used starting from 4-fluorobenzaldehyde followed by procedure F.

¹H NMR: δ 1.19 (ddd, J=1.5, 3.5, 7.7 Hz, 6H), 1.46 (ddt, J=4.9, 4.9, 6.8, 12.6 Hz, 2H), 1.57-1.66 (m, 2H), 2.49 (ddd, J=4.8, 6.7, 13.9 Hz, 2H), 2.63 (dddd, J=1.8, 4.9, 6.8, 13.6 Hz, 2H), 4.17-4.29 (m, 2H), 4.99-5.05 (m, 2H), 5.45 (dq, J=1.2, 1.2, 1.3, 6.9 Hz, 1H), 5.56 (ddt, J=1.4, 1.4, 2.8, 7.0 Hz, 1H), 6.96-7.05 (m, 4H), 7.34-7.42 (m, 2H).

### Compound 26

### (5R,6S,10bR)-6,8,9-trifluoro-5-methyl-1,5,6,10b-tetrahydropyrrolo[2,1-a]isoquinolin-3(2H)-one

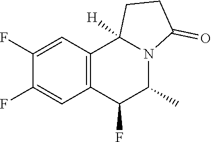 [SMILES: C[C@@H]1[C@@H](F)c2cc(F)c(F)cc2[C@H]2CCC(=O)N21]

The general procedure F was used starting from 3,4-difluorobenzaldehyde.

¹H NMR: δ 1.18 (ddd, J=1.5, 3.5, 7.5 Hz, 6H), 1.46 (ddt, J=4.9, 4.9, 6.8, 12.4 Hz, 2H), 1.57-1.66 (m, 2H), 2.49 (ddd, J=4.8, 6.7, 13.9 Hz, 2H), 2.63 (dddd, J=1.8, 4.9, 6.7, 13.6 Hz, 2H), 4.17-4.29 (m, 2H), 5.07 (ddt, J=2.3, 2.3, 3.3, 4.9 Hz, 2H), 5.52-5.58 (m, 1H), 5.63-5.68 (m, 1H), 7.06-7.13 (m, 2H), 7.14-7.22 (m, 2H).

### Compound 27

### (12bS)-8,9-difluoro-1H,2H,3H,4H,6H,7H,12H,12bH-indolo[2,3-a]quinolizin-4-one

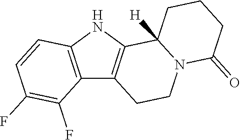 [SMILES: O=C1CCC[C@H]2c3[nH]c4ccc(F)c(F)c4c3CCN12]

The general procedure G was used starting from 4,5-difluoro-1H-indole-3-carbaldehyde.

¹H NMR: δ 1.67-1.78 (m, 1H), 2.04 (ddt, J=5.6, 8.1, 12.1 Hz, 1H), 2.21-2.35 (m, 2H), 2.40 (ddd, J=5.3, 7.9, 14.1 Hz, 1H), 2.49 (ddd, J=5.3, 7.7, 14.1 Hz, 1H), 3.86-3.97 (m, 2H), 4.00 (ddd, J=4.1, 6.1, 11.9 Hz, 1H), 4.22 (ddd, J=4.4, 5.8, 11.9 Hz, 1H), 4.84 (dd, J=2.9, 5.7 Hz, 1H), 7.07 (dd, J=4.8, 8.2 Hz, 1H), 7.14-7.21 (m, 1H), 8.56 (s, 1H).

### Compound 28

### 7,8,9-trifluoro-1H,2H,3H,4H,6H,7H,12H,12bH-indolo[2,3-a]quinolizin-4-one

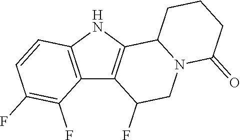 [SMILES: O=C1CCCC2c3[nH]c4ccc(F)c(F)c4c3C(F)CN12]

The general procedure G was used starting from 4,5-difluoro-1H-indole-3-carbaldehyde.

¹H NMR: δ 1.73-1.85 (m, 2H), 2.03 (ddt, J=5.5, 5.5, 8.1, 12.1 Hz, 2H), 2.22-2.44 (m, 6H), 2.49 (ddd, J=5.3, 7.7, 14.1 Hz, 2H), 3.96 (ddd, J=5.3, 12.8, 17.0 Hz, 2H), 4.29 (ddd, J=2.6, 12.8, 17.0 Hz, 2H), 4.96 (dd, J=2.7, 5.5 Hz, 2H), 6.78 (dd, J=2.6, 5.3 Hz, 1H), 6.88 (dd, J=2.6, 5.3 Hz, 1H), 7.15-7.25 (m, 4H), 8.76 (s, 2H).

### Compound 29

### (7R,12bR)-7,8,9-trifluoro-1H,2H,3H,4H,6H,7H,12H,12bH-indolo[2,3-a]quinolizin-4-one

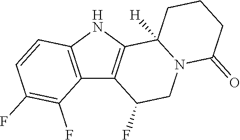 [SMILES: O=C1CCC[C@@H]2c3[nH]c4ccc(F)c(F)c4c3[C@@H](F)CN12]

The general procedure G was used starting from 4,5-difluoro-1H-indole-3-carbaldehyde.

¹H NMR: δ 1.73-1.85 (m, 2H), 2.04 (ddt, J=5.4, 5.4, 8.1, 12.1 Hz, 2H), 2.21-2.44 (m, 6H), 2.48 (ddd, J=5.3, 7.7, 14.2 Hz, 2H), 3.97 (ddd, J=5.3, 12.8, 17.0 Hz, 2H), 4.28 (ddd, J=2.7, 12.8, 17.0 Hz, 2H), 5.07 (dd, J=2.7, 5.5 Hz, 2H), 6.78 (dd, J=2.6, 5.3 Hz, 1H), 6.88 (dd, J=2.6, 5.3 Hz, 1H), 7.15-7.20 (m, 1H), 7.20-7.25 (m, 3H), 8.76 (s, 2H).

### Compound 30

### (7S,12bS)-7,8,9-trifluoro-1H,2H,3H,4H,6H, 7H,12H,12bH-indolo[2,3-a]quinolizin-4-one

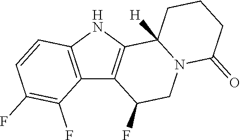 [SMILES: O=C1CCC[C@H]2c3[nH]c4ccc(F)c(F)c4c3[C@H](F)CN12]

The general procedure G was used starting from 4,5-difluoro-1H-indole-3-carbaldehyde.

¹H NMR: δ 1.73-1.85 (m, 2H), 2.03 (ddt, J=5.5, 5.5, 8.2, 12.1 Hz, 2H), 2.24-2.44 (m, 6H), 2.49 (ddd, J=5.3, 7.7, 14.1 Hz, 2H), 3.97 (ddd, J=5.3, 12.8, 17.0 Hz, 2H), 4.28 (ddd, J=2.7, 12.8, 17.2 Hz, 2H), 4.99 (dd, J=2.7, 5.5 Hz, 2H), 6.78 (dd, J=2.6, 5.3 Hz, 1H), 6.88 (dd, J=2.6, 5.3 Hz, 1H), 7.15-7.24 (m, 4H), 8.76 (s, 2H).

### Compound 31

### (7R,12bS)-7,8,9-trifluoro-1H,2H,3H,4H,6H, 7H,12H,12bH-indolo[2,3-a]quinolizin-4-one

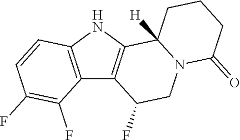 [SMILES: O=C1CCC[C@H]2c3[nH]c4ccc(F)c(F)c4c3[C@@H](F)CN12]

The general procedure G was used starting from 4,5-difluoro-1H-indole-3-carbaldehyde.

¹H NMR: δ 1.73-1.85 (m, 2H), 2.00 (ddt, J=5.5, 5.5, 8.1, 12.1 Hz, 2H), 2.24-2.43 (m, 6H), 2.49 (ddd, J=5.3, 7.6, 14.1 Hz, 2H), 3.97 (ddd, J=2.6, 12.8, 17.0 Hz, 2H), 4.29 (ddd, J=5.3, 12.8, 17.0 Hz, 2H), 5.10 (dd, J=2.7, 5.5 Hz, 2H), 6.76 (dd, J=2.7, 5.4 Hz, 1H), 6.86 (dd, J=2.6, 5.3 Hz, 1H), 7.14-7.24 (m, 4H), 8.76 (s, 2H).

### Compound 32

### (5S,6R,10bS)-6,8-difluoro-5-methyl-1,5,6,10b-tetrahydropyrrolo[2,1-a]isoquinolin-3(2H)-one

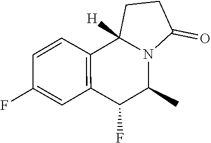 [SMILES: C[C@H]1[C@H](F)c2cc(F)ccc2[C@@H]2CCC(=O)N21]

The general procedure F was used starting from 3-fluorobenzaldehyde.

¹H NMR: δ 1.33 (d, J=6.8 Hz, 6H), 1.59-1.73 (m, 4H), 2.45 (dddd, J=1.8, 6.4, 7.3, 13.8 Hz, 2H), 2.63 (ddd, J=6.2, 7.4, 13.7 Hz, 2H), 4.26-4.36 (m, 2H), 4.96-5.04 (m, 2H), 5.39 (dd, J=0.7, 6.0 Hz, 1H), 5.50 (dd, J=0.7, 6.0 Hz, 1H), 7.00 (ddd, J=2.2, 8.1, 10.3 Hz, 2H), 7.12 (ddd, J=1.9, 2.7, 12.1 Hz, 2H), 7.25-7.32 (m, 2H).

### Compound 33

### (5S,10S,10aR)-8,10-difluoro-5-methyl-1,5,10,10a-tetrahydropyrrolo[1,2-b]isoquinolin-3(2H)-one

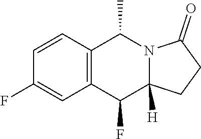 [SMILES: C[C@H]1c2ccc(F)cc2[C@H](F)[C@H]2CCC(=O)N21]

The general procedure D was used starting from 3-fluorobenzaldehyde.

¹H NMR: δ 1.21 (ddd, J=1.6, 3.6, 7.5 Hz, 6H), 1.71-1.83 (m, 2H), 1.96 (ddt, J=6.2, 6.2, 9.0, 12.5 Hz, 2H), 2.07-2.21 (m, 4H), 2.32 (ddd, J=5.4, 8.2, 14.0 Hz, 2H), 2.45 (ddd, J=5.5, 8.0, 13.8 Hz, 2H), 4.06-4.17 (m, 2H), 4.88-4.95 (m, 2H), 5.45-5.51 (m, 1H), 5.59 (ddt, J=1.1, 1.1, 2.1, 4.0 Hz, 1H), 6.99-7.11 (m, 4H), 7.20-7.26 (m, 2H).

### Compound 34

### (1R,4R)-4,6,7-trifluoro-1-methyl-2,3,4,9-tetrahydro-1H-pyrido[3,4-b]indole

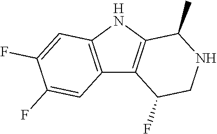 [SMILES: C[C@H]1NC[C@H](F)c2c1[nH]c1cc(F)c(F)cc21]

The general procedure H was used starting from 5,6-difluoro-1H-indole-3-carbaldehyde.

¹H NMR: δ 1.60 (s, 6H), 3.31-3.42 (m, 2H), 3.55 (dddd, J=1.8, 5.3, 13.7, 17.2 Hz, 2H), 4.01 (dtd, J=3.3, 5.3, 5.3, 6.2 Hz, 2H), 4.52 (dq, J=3.8, 3.8, 3.9, 6.4 Hz, 2H), 6.75 (dd, J=1.8, 4.6 Hz, 1H), 6.85 (dd, J=1.8, 4.6 Hz, 1H), 7.15 (dd, J=4.2, 12.1 Hz, 2H), 7.35-7.41 (m, 2H), 8.68 (s, 2H).

### Compound 35

### (7R,12bS)-7,9,10-trifluoro-1H,2H,3H,4H,6H,7H,12H,12bH-indolo[2,3-a]quinolizin-4-one

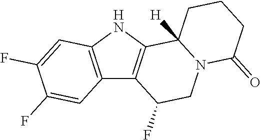 [SMILES: O=C1CCC[C@H]2c3[nH]c4cc(F)c(F)cc4c3[C@@H](F)CN12]

The general procedure G was used starting from 5,6-difluoro-1H-indole-3-carbaldehyde.

¹H NMR: δ 1.84-2.08 (m, 6H), 2.22-2.35 (m, 4H), 2.44 (ddd, J=5.8, 8.4, 14.8 Hz, 2H), 2.48-2.60 (m, 4H), 2.88-2.97 (m, 2H), 3.45 (ddd, J=2.7, 5.5, 6.8 Hz, 2H), 6.75 (ddd, J=2.5, 4.9, 7.4 Hz, 1H), 6.85 (ddd, J=2.5, 4.9, 7.6 Hz, 1H), 7.11-7.17 (m, 2H), 7.59 (dd, J=4.7, 12.1 Hz, 2H), 8.54 (s, 2H).

### Compound 36

### (7R,12bR)-7,8-difluoro-1H,2H,3H,4H,6H,7H,12H,12bH-indolo[2,3-a]quinolizin-4-one

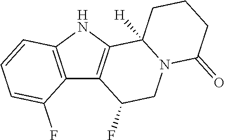 [SMILES: O=C1CCC[C@@H]2c3[nH]c4cccc(F)c4c3[C@@H](F)CN12]

The general procedure G was used starting from 4-fluoro-1H-indole-3-carbaldehyde.

¹H NMR: δ 1.73-1.85 (m, 2H), 2.03 (ddt, J=5.4, 5.4, 8.1, 12.1 Hz, 2H), 2.22-2.44 (m, 6H), 2.49 (ddd, J=5.3, 7.8, 14.2 Hz, 2H), 3.95 (ddd, J=5.3, 12.8, 17.0 Hz, 2H), 4.26 (ddd, J=2.6, 12.8, 17.0 Hz, 2H), 4.96 (dd, J=2.7, 5.5 Hz, 2H), 6.78 (dd, J=2.6, 5.3 Hz, 1H), 6.88 (dd, J=2.7, 5.4 Hz, 1H), 7.16 (ddd, J=2.7, 7.1, 10.1 Hz, 2H), 7.26 (dd, J=2.6, 12.1 Hz, 2H), 7.29-7.35 (m, 2H), 8.81 (s, 2H).

### Compound 37

### (5R,6R,10bS)-6,9-difluoro-5-methyl-1,5,6,10b-tetrahydropyrrolo[2,1-a]isoquinolin-3(2H)-one

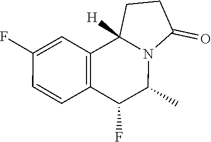 [SMILES: C[C@@H]1[C@H](F)c2ccc(F)cc2[C@@H]2CCC(=O)N12]

The general procedure F was used starting from 4-fluorobenzaldehyde.

¹H NMR: δ 1.19 (ddd, J=1.5, 3.5, 7.7 Hz, 3H), 1.46 (ddt, J=4.9, 4.9, 6.8, 12.5 Hz, 1H), 1.57-1.66 (m, 1H), 2.49 (dddd, J=1.8, 4.9, 6.7, 13.8 Hz, 1H), 2.63 (ddd, J=4.8, 6.7, 13.9 Hz, 1H), 4.25 (dtdd, J=2.6, 4.0, 7.5, 7.5, 15.2 Hz, 1H), 5.00-5.06 (m, 1H), 6.96-7.05 (m, 2H), 7.35-7.42 (m, 1H).

### Compound 38

### 6-Fluoro-1-methyl-4,9-dihydro-3H-pyrido[3,4-b]indole

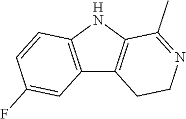

The general procedure H was used starting from 5-fluoro-1H-indole-3-carbaldehyde.

¹H NMR: δ 1.96 (s, 2H), 2.24 (dddd, J=4.8, 6.6, 9.3, 13.4 Hz, 1H), 2.49 (dddd, J=4.8, 6.4, 9.1, 13.2 Hz, 1H), 3.40-3.51 (m, 2H), 3.57 (ddd, J=6.6, 9.2, 11.0 Hz, 1H), 3.75 (d, J=5.9 Hz, 1H), 4.51 (tq, J=1.5, 6.0 Hz, 1H), 5.88 (dd, J=4.6, 7.8 Hz, 1H), 6.82 (ddd, J=2.2, 7.8, 10.1 Hz, 1H), 6.92-6.99 (m, 1H).

### Compound 39

### (7R,12bS)-7,8,9,10-Tetrafluoro-1H,2H,3H,4H,6H,7H,12H,12bH-indolo[2,3-a]quinolizin-4-one

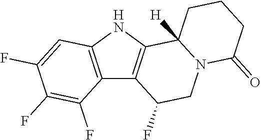 [SMILES: O=C1CCC[C@H]2c3[nH]c4cc(F)c(F)c(F)c4c3[C@@H](F)CN12]

The general procedure G was used starting from 4,5,6-trifluoro-1H-indole-3-carbaldehyde.

¹H NMR: δ 1.73-1.85 (m, 2H), 2.07 (ddt, J=5.4, 5.4, 8.1, 11.9 Hz, 2H), 2.20-2.44 (m, 6H), 2.49 (ddd, J=5.3, 7.7, 14.2 Hz, 2H), 3.97 (ddd, J=2.6, 12.7, 17.0 Hz, 2H), 4.28 (ddd, J=5.5, 12.8, 17.0 Hz, 2H), 4.95 (dd, J=2.7, 5.5 Hz, 2H), 6.73-6.82 (m, 3H), 6.86 (dd, J=2.6, 5.3 Hz, 1H), 9.05 (s, 2H).

### Compound 40

### (7R,12bR)-7,8,9,10-Tetrafluoro-1H,2H,3H,4H,6H, 7H,12H,12bH-indolo[2,3-a]quinolizin-4-one

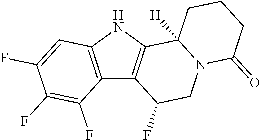 [SMILES: O=C1CCC[C@@H]2c3[nH]c4cc(F)c(F)c(F)c4c3[C@@H](F)CN12]

The general procedure G was used starting from 4,5,6-trifluoro-1H-indole-3-carbaldehyde.

¹H NMR: δ 1.73-1.85 (m, 2H), 2.04 (ddt, J=5.5, 5.5, 8.0, 12.1 Hz, 2H), 2.21-2.53 (m, 8H), 3.97 (ddd, J=5.4, 12.8, 17.0 Hz, 2H), 4.28 (ddd, J=2.6, 12.8, 17.0 Hz, 2H), 4.96 (dd, J=2.7, 5.5 Hz, 2H), 6.75-6.82 (m, 3H), 6.88 (dd, J=2.6, 5.3 Hz, 1H), 9.05 (s, 2H).

### Compound 41

### (7S,12bR)-7,8,9,10-Tetrafluoro-1H,2H,3H,4H,6H, 7H,12H,12bH-indolo[2,3-a]quinolizin-4-one

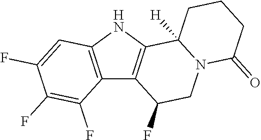 [SMILES: O=C1CCC[C@@H]2c3[nH]c4cc(F)c(F)c(F)c4c3[C@H](F)CN12]

The general procedure G was used starting from 4,5,6-trifluoro-1H-indole-3-carbaldehyde.

¹H NMR: δ 1.73-1.85 (m, 2H), 2.07 (ddt, J=5.4, 5.4, 8.1, 11.9 Hz, 2H), 2.20-2.44 (m, 6H), 2.49 (ddd, J=5.3, 7.7, 14.2 Hz, 2H), 3.97 (ddd, J=2.6, 12.7, 17.0 Hz, 2H), 4.28 (ddd, J=5.5, 12.8, 17.0 Hz, 2H), 4.92 (dd, J=2.7, 5.5 Hz, 2H), 6.73-6.82 (m, 3H), 6.86 (dd, J=2.6, 5.3 Hz, 1H), 9.05 (s, 2H).

### Compound 42

### (5S,10R,10aR)-7,8,10-Trifluoro-5-methyl-1,5,10,10a-tetrahydropyrrolo[1,2-b]isoquinolin-3(2H)-one

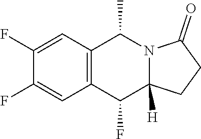 [SMILES: C[C@H]1c2cc(F)c(F)cc2[C@@H](F)[C@H]2CCC(=O)N21]

The general procedure D was used starting from 5-[(R)-(3,4-difluorophenyl)(fluoro)methyl]pyrrolidin-2-one.

¹H NMR: δ 1.37 (s, 6H), 1.69 (ddtd, J=3.3, 5.8, 5.8, 7.7, 11.7 Hz, 2H), 1.88-1.98 (m, 2H), 2.44 (dddd, J=1.8, 6.0, 7.9, 13.9 Hz, 2H), 2.62 (ddd, J=6.0, 7.8, 13.8 Hz, 2H), 4.22-4.32 (m, 2H), 4.96 (dtd, J=2.4, 6.3, 6.3, 7.0 Hz, 2H), 5.72-5.76 (m, 1H), 5.85 (dd, J=0.7, 3.3 Hz, 1H), 7.02-7.08 (m, 2H), 7.08-7.16 (m, 2H).

### Compound 43

### (5S,10R,10aS)-7,8,10-Trifluoro-5-methyl-1,5,10,10a-tetrahydropyrrolo[1,2-b]isoquinolin-3(2H)-one

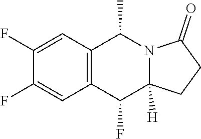 [SMILES: C[C@H]1c2cc(F)c(F)cc2[C@@H](F)[C@@H]2CCC(=O)N12]

The general procedure D was used starting from (5S)-5-[(R)-(3,4-difluorophenyl)(fluoro)methyl]pyrrolidin-2-one.

¹H NMR: δ 1.39 (s, 6H), 4.76 (dtdd, J=1.8, 3.8, 5.8, 5.8, 16.3 Hz, 2H), 5.07-5.15 (m, 2H), 5.68-5.74 (m, 1H), 5.82 (ddd, J=0.7, 1.8, 6.0 Hz, 1H), 6.12 (dd, J=1.7, 9.4 Hz, 2H), 6.90 (dddd, J=1.8, 3.7, 5.5, 9.2 Hz, 2H), 7.05-7.12 (m, 2H), 7.25-7.33 (m, 2H).

### Compound 44

### (5S,10S,10aR)-7,8,10-Trifluoro-5-methyl-1,5,10,10a-tetrahydropyrrolo[1,2-b]isoquinolin-3(2H)-one

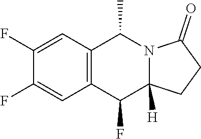 [SMILES: C[C@H]1c2cc(F)c(F)cc2[C@H](F)[C@H]2CCC(=O)N21]

The general procedure D was used starting from (5R)-5-[(S)-(3,4-difluorophenyl)(fluoro)methyl]pyrrolidin-2-one.

¹H NMR: δ 1.39 (s, 6H), 1.68 (ddtd, J=3.3, 5.8, 5.8, 7.7, 11.9 Hz, 2H), 1.88-1.98 (m, 2H), 2.45 (dddd, J=1.8, 6.0, 7.8, 13.9 Hz, 2H), 2.63 (ddd, J=5.9, 7.7, 13.6 Hz, 2H), 4.30 (dtdt, J=2.2, 2.2, 4.2, 6.0, 6.0, 12.0 Hz, 2H), 4.91-5.00 (m, 2H), 5.49 (dd, J=0.7, 6.0 Hz, 1H), 5.59 (d, J=6.0 Hz, 1H), 7.04-7.11 (m, 2H), 7.16-7.24 (m, 2H).

### Compound 45

### (12bS)-9,10-difluoro-1H,2H,3H,4H,6H,7H,12H,12bH-indolo[2,3-a]quinolizin-4-one

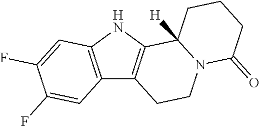 [SMILES: O=C1CCC[C@H]2c3[nH]c4cc(F)c(F)cc4c3CCN12]

The general procedure B was used starting from 5,6-difluoro-1H-indole-3-carbaldehyde, followed by general procedure G.

¹H NMR: δ 1.39 (s, 6H), 1.68 (ddtd, J=3.3, 5.8, 5.8, 7.7, 11.9 Hz, 2H), 1.88-1.98 (m, 2H), 2.45 (dddd, J=1.8, 6.0, 7.8, 13.9 Hz, 2H), 2.63 (ddd, J=5.9, 7.7, 13.6 Hz, 2H), 4.30 (dtdt, J=2.2, 2.2, 4.2, 6.0, 6.0, 12.0 Hz, 2H), 4.91-5.00 (m, 2H), 5.49 (dd, J=0.7, 6.0 Hz, 1H), 5.59 (d, J=6.0 Hz, 1H), 7.04-7.11 (m, 2H), 7.16-7.24 (m, 2H).

### Experiments Relating to Binding Affinities of the Compounds of the Invention

Calculations of binding affinities of the compounds of the invention were performed using Qvina2 using the value 5061982 for explicit random seed. Exhaustiveness was 100. The method of performing binding affinity—calculations of the compounds of the invention is well known in the art and also described in Alhossary A. et al. Fast, Accurate, and Reliable Molecular Docking with QuickVina 2 Bioinformatics (2015) 31 (13) 2214-2216, 0. Trott, A. J. Olson, AutoDock Vina: improving the speed and accuracy of docking with a new scoring function, efficient optimization and multithreading, Journal of Computational Chemistry 31 (2010) 455-461, Feinstein W P, Brylinski M. Calculating an optimal box size for ligand docking and virtual screening against experimental and predicted binding pockets. J. Cheminform. (2015), 7 (1): 18, Tetko, I. V. et al. Virtual computational chemistry laboratory—design and description, J. Comput. Aid. Mol. Des., 2005, 19, 453-63, VCCLAB, Virtual Computational Chemistry Laboratory, http://www.vcclab.org, 2005. The search space (X,Y,Z)-coordinates of the centre for the calculations of binding affinities can be obtained with methods well known in the art. E.g. the search space (X,Y,Z)-coordinates for PDB ID 1bp3 and protein homolog ClassA_5 ht1a_human_Active_6 G79_2018-07-10_GPCRDB were (13.730, 30.880, 13.170) and (91.84457623, 54.99481, 63.31365), respectively. The PDB IDs and the protein homolog codes of proteins used in the calculations of the compounds of the invention are listed in Table 1 and results of the calculations are presented in Table 2.

TABLE 1
The PDB IDs and the protein homolog codes of
proteins used in the calculations of binding
affinities of the compounds of the invention.

| | PDB | | |
|---|---|---|---|
| Entry | ID¹ | Entry | Homolog² |
| 1 | 1bp3 | 42 | ClassA_5ht1a_human_Active_6G79_2018- |
| | | | 07-10_GPCRDB |
| 2 | 1h22 | 43 | ClassA_5ht2a_human_Active_6BQG_2018- |
| | | | 07-10_GPCRDB |
| 3 | 1kux | 44 | ClassA_ada1a_human_Active_3SN6_2018- |
| | | | 07-10_GPCRDB |
| 4 | 1n7i | 45 | ClassA_ada1b_human_Active_3SN6_2018- |
| | | | 07-10_GPCRDB |
| 5 | 2toh | 46 | ClassA_ada2a_human_Active_6G79_2018- |
| | | | 07-10_GPCRDB |
| 6 | 2vz2 | 47 | ClassA_ada2b_human_Active_6G79_2018- |
| | | | 07-10_GPCRDB |
| 7 | 2xsn | 48 | ClassA_ada2c_human_Active_6G79_2018- |
| | | | 07-10_GPCRDB |
| 8 | 2z5x | 49 | ClassA_hrh3_human_Active_4MQT_2018- |
| | | | 07-10_GPCRDB |
| 9 | 3bwm | 50 | ClassA_mtr1a_human_Active_3SN6_2018- |
| | | | 07-10_GPCRDB |
| 10 | 3hcd | 51 | ClassA_mtr1b_human_Active_5TUD_2018- |
| | | | 07-10_GPCRDB |
| 11 | 3pbl | 52 | ClassA_taar1_human_Active_3SN6_2018- |
| | | | 07-10_GPCRDB |
| 12 | 3qel | 53 | ClassA_drd1_human_Active_3SN6_2018- |
| | | | 07-10_GPCRDB |
| 13 | 3rze | 54 | NET_SLC6A2 |
| 14 | 3uon | 55 | neuropeptide_y2_receptor_2ik3 |
| 15 | 3zpq | 56 | neuropeptide_y5_receptor_2he6 |
| 16 | 4a6e | 57 | ClassA_acthr_human_Active_5XRA_2018- |
| | | | 07-10_GPCRDB |
| 17 | 4daj | 58 | ClassA_mc3r_human_Active_5XRA_2018- |
| | | | 07-10_GPCRDB |
| 18 | 4djh | 59 | ClassA_mc4r_human_Active_5XRA_2018- |
| | | | 07-10_GPCRDB |
| 19 | 4kfq | 60 | ClassA_mc5r_human_Active_5XRA_2018- |
| | | | 07-10_GPCRDB |
| 20 | 4ldo | 61 | ClassA_mshr_human_Active_5XRA_2018- |
| | | | 07-10_GPCRDB |
| 21 | 4n6h | 62 | ClassA_nk1r_human_Active_6B73_2018- |
| | | | 07-10_GPCRDB |
| 22 | 4rvm | 63 | ClassA_nk2r_human_Active_6B73_2018- |
| | | | 07-10_GPCRDB |
| 23 | 4s0v | 64 | ClassA_nk3r_human_Active_6B73_2018- |
| | | | 07-10_GPCRDB |
| 24 | 4xp1 | 65 | ClassA_oxyr_human_Active_5DYS_2018- |
| 25 | 4zj8 | | 07-10_GPCRDB |
| 26 | 5ain | | |
| 27 | 5c1m | | |
| 28 | 5cxv | | |
| 29 | 5dhh | | |
| 30 | 5dsg | | |
| 31 | 5hk1 | | |
| 32 | 5i73 | | |
| 33 | 5kxi | | |
| 34 | 5pah | | |
| 35 | 5tvn | | |
| 36 | 5v54 | | |
| 37 | 5wiu | | |
| 38 | 6bqg | | |
| 39 | 6cm4 | | |
| 40 | 4k5y | | |
| 41 | 5zbq | | |

¹The PDB structures were obtained from the Protein Data Bank: www.rcsb.org/.
²Protein homologs were obtained from GPCRdb (http://docs.gpcrdb.org/index.html, Pándy-Szekeres G, Munk C, Tsonkov T M, Mordalski S, Harpsøe K, Hauser A S, Bojarski A J, Gloriam D E. GPCRdb in 2018: adding GPCR structure models and ligands. 2017, Nucleic Acids Res., Nov 16. http://dx.doi.org/10.1093/nar/gkx1109).

TABLE 2
Results of binding affinity calculations of compounds of the
invention performed on proteins, logP and logS values.

| | | | Search | | | | | | |
|---|---|---|---|---|---|---|---|---|---|
| | | | space | | | | | | |
| | | | (x.x.x) | | | | | | |
| | | | in Å | | | | | | |
| | | | Value* | Docked protein¹ | | | | | |
| | | | Value | 52 | 24 | 53 | 39 | 11 | 37 |
| Compound | logP | logS | *Value | ΔG (kcal/mol) | | | | | |
| 1 | 2.07 | −2.81 | 11.385 | −7.7 | −7.1 | −7.8 | −7.3 | −6.9 | −7.9 |
| 2 | 2.19 | −3.07 | 11.960 | −7.9 | −8.1 | −7.8 | −7.8 | −7.1 | −7.7 |
| 3 | 2.64 | −3.31 | 11.249 | −8.1 | −8.1 | −7.3 | −7.4 | −7.4 | −7.6 |
| 4 | 2.06 | −3.11 | 11.960 | −8.2 | −8.5 | −8 | −8.1 | −7.2 | −8.2 |
| 5 | 2.46 | −2.24 | 13.339 | −8.8 | −7.5 | −8.6 | −8.5 | −8.4 | −9.7 |
| 6 | 2.58 | −2.88 | 14.195 | −9.1 | −8.2 | −8.5 | −8.7 | −8.8 | −9.1 |
| 7 | 2.51 | −2.93 | 14.851 | −9.6 | −7.7 | −7.6 | −9 | −8.8 | −10.6 |
| 8 | 2.61 | −3.04 | 13.120 | −9 | −8.5 | −8.9 | −7.4 | −7.1 | −9.2 |
| 9 | 2.62 | −3.18 | 14.277 | −9.3 | −8.5 | −8.8 | −8.9 | −9.1 | −10 |
| 10 | 2.17 | −3.76 | 12.847 | −8.9 | −7.7 | −8.3 | −8.4 | −7.5 | −8.9 |
| 11 | 2.51 | −3.38 | 12.106 | −8.8 | −7.2 | −7.8 | −7.8 | −7.3 | −8.5 |
| 12 | 2.09 | −3.67 | 12.661 | −8.5 | −7.1 | −7.7 | −7.8 | −7.3 | −8.5 |
| 13 | 2.48 | −3.45 | 12.699 | −8.6 | −7.1 | −7.9 | −8.2 | −7.5 | −8.9 |
| 14 | 2.34 | −2.37 | 12.513 | −8.5 | −7.4 | −8.4 | −8.4 | −7.4 | −8.4 |
| 15 | 2.24 | −2.00 | 11.945 | −8.3 | −7.1 | −8.5 | −8.1 | −7.2 | −8.8 |
| 16 | 2.26 | −2.02 | 11.731 | −9.5 | −7.5 | −8.2 | −8.2 | −7.5 | −8.4 |
| 17 | 2.25 | −2.76 | 12.828 | −8.6 | −8.1 | −8.4 | −8.2 | −7.5 | −8.5 |
| 18 | 2.48 | −2.28 | 12.769 | −9.2 | −7.9 | −7.4 | −9.3 | −8.2 | −9.7 |
| 19 | 2.98 | −2.65 | 13.536 | −9.3 | −7.4 | −7.2 | −9.2 | −8.9 | −9.8 |
| 20 | 2.61 | −2.21 | 12.632 | −9 | −7.5 | −7.3 | −8.9 | −8.4 | −9.3 |
| 21 | 3.18 | −2.84 | 13.794 | −9.6 | −7.2 | −6.8 | −9.6 | −8.2 | −10.3 |
| 22 | 2.81 | −2.80 | 14.088 | −9 | −7.6 | −7.5 | −9.4 | −8.5 | −9.4 |
| 23 | 3.01 | −3.25 | 14.575 | −9.9 | −7.8 | −7.9 | −9.5 | −8.7 | −9.9 |

| | Docked protein¹ | | | | | | | | |
|---|---|---|---|---|---|---|---|---|---|
| | 54 | 10 | 4 | 32 | 3 | 16 | 42 | 43 | 36 |
| Compound | ΔG (kcal/mol) | | | | | | | | |
| 1 | −8 | −7 | −7.8 | −8.4 | −6.3 | −6.7 | −7.1 | −8.7 | −7.6 |
| 2 | −7.8 | −8.1 | −8.2 | −8.6 | −6.3 | −7.8 | −7.3 | −8.8 | −7.7 |
| 3 | −7.4 | −7.1 | −7.2 | −8.1 | −5.8 | −7.2 | −7 | −7.7 | −7.8 |
| 4 | −8.1 | −7.6 | −7.9 | −8.7 | −6.6 | −8.2 | −7.4 | −9.1 | −8.3 |
| 5 | −8.5 | −8.9 | −7.2 | −9.3 | −7 | −7.6 | −8.3 | −10.3 | −10.1 |
| 6 | −8.8 | −9.6 | −7.3 | −9.6 | −7.4 | −8.1 | −8 | −9.1 | −10.1 |
| 7 | −8.9 | −6.7 | −4.2 | −10.6 | −8.2 | −7.5 | −9.1 | −10.4 | −9.4 |
| 8 | −8.6 | −2.2 | −4.2 | −9.8 | −7.5 | −8.6 | −8.5 | −8.7 | −8.9 |
| 9 | −9.1 | −7.7 | −5.1 | −9.8 | −7.7 | −8.1 | −8.8 | −10 | −9.7 |
| 10 | −8.4 | −6.7 | −6.7 | −9.6 | −7.2 | −7.9 | −8 | −9.9 | −8.7 |
| 11 | −8.1 | −6.6 | −7 | −8.8 | −6.3 | −7.5 | −7.5 | −8.3 | −7.9 |
| 12 | −8.2 | −7.1 | −6.4 | −9.2 | −6.7 | −7.7 | −8.2 | −8.7 | −8 |
| 13 | −8.5 | −7.2 | −7 | −9.2 | −7 | −8 | −8.4 | −8.9 | −8.5 |
| 14 | −8.6 | −7.6 | −8.5 | −8.8 | −7.3 | −8.6 | −8.1 | −8.5 | −8.9 |
| 15 | −9.4 | −7.3 | −8.4 | −8.4 | −6.8 | −8.2 | −7.3 | −8.3 | −8.5 |
| 16 | −8.9 | −6.6 | −7.6 | −8.5 | −6.8 | −8.6 | −8 | −8.2 | −8.8 |
| 17 | −8.3 | −8.3 | −8.3 | −9 | −7.2 | −8.5 | −8.1 | −8.6 | −9.2 |
| 18 | −9 | −3.7 | −3.7 | −10.3 | −7.4 | −8.6 | −8.8 | −9.5 | −9.3 |
| 19 | −8.5 | −5.8 | −4.1 | −9.8 | −7.5 | −7.8 | −9 | −8.5 | −9.5 |
| 20 | −8.7 | −5.7 | −5.2 | −9.7 | −7.1 | −8.3 | −8.6 | −9 | −9.5 |
| 21 | −8.7 | −2.9 | −0.6 | −10.4 | −7.7 | −7.3 | −9 | −9 | −10 |
| 22 | −8.4 | −7.4 | −4.3 | −10.3 | −7.3 | −7.7 | −8.1 | −9.5 | −9.4 |
| 23 | −8.5 | −5.7 | −3.1 | −10.6 | −7.5 | −8 | −8.7 | −9.3 | −9.6 |
| | Docked protein¹ | | | | | | | | |
| | 35 | 38 | 26 | 8 | 6 | 9 | 44 | 45 | 46 |
| Compound | ΔG (kcal/mol) | | | | | | | | |
| 1 | −7.7 | −8.6 | −8 | −8 | −7.9 | −5.8 | −7.1 | −7.3 | −5.5 |
| 2 | −7.5 | −8.8 | −8.3 | −7.6 | −8.1 | −5.7 | −7.1 | −7.2 | −5.4 |
| 3 | −7.4 | −7.7 | −8.4 | −8.9 | −7.9 | −5.6 | −7 | −6.9 | −5 |
| 4 | −7.6 | −9.1 | −7.5 | −7.4 | −7.9 | −5.9 | −7.4 | −7.5 | −5.5 |
| 5 | −9 | −10.2 | −5.1 | −8.6 | −6.8 | −6.5 | −7.7 | −7.2 | −6.4 |
| 6 | −9.4 | −9 | −5.3 | −10.6 | −7.2 | −6.6 | −8.3 | −7.8 | −6.6 |
| 7 | −10 | −9.6 | −3.6 | −10.1 | −6 | −6.9 | −8.7 | −8 | −7.3 |
| 8 | −8.9 | −8.1 | −4.2 | −4.8 | −4.3 | −6.4 | −8 | −6.8 | −6 |
| 9 | −9.5 | −9.9 | −3.4 | −9.6 | −6.7 | −7.2 | −8.4 | −7.7 | −6.9 |
| 10 | −8.3 | −9.9 | −7.7 | −7.8 | −7 | −6.4 | −7.6 | −8.1 | −6.2 |
| 11 | −8.2 | −8.3 | −8.9 | −9.3 | −8.8 | −5.8 | −7.2 | −7.2 | −5.4 |
| 12 | −8.2 | −8.6 | −8.3 | −9.5 | −8.7 | −6.3 | −7.3 | −7.4 | −5.6 |
| 13 | −8.5 | −8.8 | −8.3 | −9.4 | −8.5 | −6.1 | −7.4 | −7.7 | −5.8 |
| 14 | −8.2 | −7.9 | −7.1 | −7.7 | −5.8 | −6.4 | −7.8 | −7.8 | −5.9 |
| 15 | −7.9 | −8 | −6.8 | −7.4 | −6.6 | −6.2 | −7.6 | −7.6 | −5.4 |
| 16 | −8.2 | −8.2 | −6.2 | −7.6 | −6.6 | −5.9 | −7 | −7.5 | −5.2 |
| 17 | −8.2 | −8.4 | −8.5 | −8.5 | −6.8 | −6.1 | −7.6 | −8.2 | −5.8 |
| 18 | −9 | −9.5 | −8.5 | −9 | −5 | −6.7 | −9.1 | −7.8 | −6.1 |
| 19 | −9.4 | −7.9 | −6 | −9.3 | −4 | −6.5 | −8.4 | −7 | −7 |
| 20 | −8.7 | −8.9 | −8.2 | −9.2 | −5.3 | −6.8 | −8.8 | −7.7 | −6.2 |
| 21 | −9.6 | −9 | −4.9 | −9.9 | −3.8 | −6.6 | −8.5 | −6.9 | −7.2 |
| 22 | −9.4 | −8.6 | −8.1 | −9.8 | −4.2 | −6.5 | −8.4 | −7.5 | −6.9 |
| 23 | −9.6 | −8.5 | −6.7 | −9.6 | −2.8 | −7.1 | −8.7 | −6.5 | −7.1 |
| | Docked protein¹ | | | | | | | | |
| | 47 | 48 | 15 | 20 | 34 | 7 | 5 | 13 | 49 |
| Compound | ΔG (kcal/mol) | | | | | | | | |
| 1 | −6.9 | −1.9 | −8 | −6.7 | −8.1 | 3.5 | −8.5 | −6.8 | −6.5 |
| 2 | −6.9 | −2.3 | −8.1 | −6.1 | −8.1 | 0.2 | −8.6 | −6.7 | −6.7 |
| 3 | −7 | −2.8 | −8.3 | −6.6 | −7.6 | 0.9 | −8.1 | −7.4 | −6.6 |
| 4 | −7.1 | −1.8 | −8.1 | −5.4 | −8.3 | −0.4 | −9 | −6.9 | −6.9 |
| 5 | −8 | −1.3 | −7.6 | −7 | −8.7 | 2.1 | −8.8 | −9.2 | −6.5 |
| 6 | −7.9 | −1.8 | −7.8 | −6.8 | −8.7 | −1.6 | −9 | −9.6 | −7.1 |
| 7 | −8.3 | −2 | −8.9 | −5.8 | −9 | −0.8 | −9.7 | −7.8 | −7.1 |
| 8 | −7.6 | −1.1 | −8.4 | −5.1 | −8.8 | 4.5 | −8.9 | −5.3 | −6.6 |
| 9 | −8.2 | −1.8 | −8.1 | −6.7 | −8.6 | −0.7 | −9.3 | −8.4 | −6.8 |
| 10 | −7.3 | −2.1 | −8.6 | −5.8 | −8.7 | 0 | −9 | −6.9 | −7.3 |
| 11 | −7.4 | −2.8 | −8.9 | −6.2 | −8.1 | 1.6 | −8.4 | −6.8 | −6.7 |
| 12 | −7.3 | −1.9 | −8.7 | −6.5 | −8.5 | −0.3 | −8.7 | −6.3 | −6.3 |
| 13 | −7.6 | −2 | −8.8 | −7.5 | −8.9 | 4.3 | −9.1 | −6.4 | −6.4 |
| 14 | −7.7 | −1.5 | −8.8 | −6.8 | −8.8 | 6.4 | −9.3 | −9.8 | −7 |
| 15 | −7.6 | −1.1 | −8.4 | −4.9 | −8.6 | 12.5 | −8.7 | −9.5 | −6.4 |
| 16 | −7.9 | −1.4 | −8.9 | −5.9 | −8.8 | 13 | −8.5 | −7.5 | −6.8 |
| 17 | −7.4 | −2.4 | −8.9 | −7 | −8.5 | 2 | −8.8 | −7.8 | −6.8 |
| 18 | −7.7 | 1.3 | −7.6 | −3.5 | −9.3 | 5.7 | −9.2 | −7.8 | −7.1 |
| 19 | −8.1 | −0.7 | −8.7 | −7.6 | −9.5 | 4 | −9.6 | −7.8 | −6.7 |
| 20 | −7.7 | 0.1 | −7.3 | −5 | −8.8 | 8.1 | −8.9 | −8.5 | −7.1 |
| 21 | −8.5 | 0.4 | −9.2 | −6.2 | −9.9 | 6.3 | −10.2 | −6.9 | −7 |
| 22 | −7.5 | −3.4 | −9.2 | −8.6 | −8.9 | 0.8 | −9.6 | −7.2 | −6.7 |
| 23 | −7.9 | −3.1 | −9.5 | −8.8 | −9.5 | 1.2 | −10.3 | −6.6 | −6.8 |
| | Docked protein¹ | | | | | | | | |
| | 25 | 23 | 1 | 19 | 12 | 33 | 31 | 29 | 27 |
| Compound | ΔG (kcal/mol) | | | | | | | | |
| 1 | −7.1 | −7.4 | −4.2 | −7.5 | −4.7 | −5.8 | −8.4 | −5.9 | −7.2 |
| 2 | −7.2 | −7.5 | −4.1 | −7.7 | −5 | −6.7 | −8.7 | −5.9 | −7.4 |
| 3 | −6.9 | −7.1 | −3.6 | −7.9 | −4.5 | −6.7 | −8.3 | −5.7 | −7 |
| 4 | −7.6 | −7.8 | −4.1 | −7.3 | −4.9 | −6 | −8.9 | −6.2 | −7.8 |
| 5 | −9 | −9.3 | −4.6 | −2.6 | −2.2 | 0.3 | −10.2 | −7 | −8.5 |
| 6 | −8.8 | −8.8 | −4.9 | −2.2 | −3.6 | −1.9 | −10.7 | −7.5 | −8.5 |
| 7 | −9.2 | −9.8 | −5.3 | −1.4 | −3.1 | −0.2 | −11 | −7.7 | −9.3 |
| 8 | −8.8 | −9.1 | −5.6 | −5.8 | 2.3 | −0.6 | −9.2 | −7.5 | −8.8 |
| 9 | −9.2 | −9.5 | −5.1 | −2.4 | −4.8 | 0.3 | −10.9 | −7.5 | −8.9 |
| 10 | −8.1 | −8.2 | −4.9 | −6.8 | −4.8 | −6.2 | −9.5 | −6.6 | −8.2 |
| 11 | −7.3 | −7.6 | −4.2 | −7.8 | −5.3 | −5 | −8.9 | −6.2 | −7.4 |
| 12 | −7.7 | −7.7 | −4.7 | −7.6 | −5.6 | −4.8 | −9.1 | −6.6 | −7.8 |
| 13 | −8 | −7.7 | −4.8 | −8 | −4.7 | −4.3 | −9.1 | −6.8 | −8 |
| 14 | −8 | −8.3 | −4.7 | −4.1 | −4.6 | −2.8 | −9 | −6.7 | −8 |
| 15 | −7.8 | −8 | −4.6 | −4.9 | −3.3 | −1.2 | −8.7 | −6.6 | −7.7 |
| 16 | −8.1 | −8 | −3.9 | −6.4 | −1.4 | −1.3 | −8.5 | −6.3 | −7.8 |
| 17 | −7.8 | −7.8 | −4.4 | −6.4 | −5.7 | −3.9 | −9 | −6.3 | −7.9 |
| 18 | −8.5 | −9.4 | −6.1 | −5 | −2.6 | −0.3 | −9.5 | −7.1 | −8.9 |
| 19 | −9 | −9.3 | −5.2 | −5.1 | −5 | −3.4 | −9.5 | −7.2 | −9.1 |
| 20 | −8.4 | −9.1 | −5.8 | −6.6 | −3 | −1.4 | −9 | −6.7 | −8.7 |
| 21 | −9.2 | −9.2 | −4.9 | −3.7 | −3.1 | −1.1 | −9.9 | −7.4 | −9.2 |
| 22 | −8.4 | −8.5 | −4.6 | −5 | −5.3 | −5.8 | −9.8 | −7.2 | −8.9 |
| 23 | −8.8 | −8.9 | −4.8 | −3 | −4.9 | −5.8 | −10.1 | −7.4 | −9.3 |
| | Docked protein¹ | | | | | | | | |
| | 21 | 18 | 2 | 22 | 28 | 14 | 17 | 30 | 41 |
| Compound | ΔG (kcal/mol) | | | | | | | | |
| 1 | −7.6 | −7.6 | −8.5 | −7.8 | −8.6 | −8.2 | −8.4 | −8.7 | −7.3 |
| 2 | −7.7 | −7.2 | −8.8 | −7.8 | −8.8 | −8.5 | −8.4 | −9.1 | −7.4 |
| 3 | −7.3 | −7 | −8.2 | −7.6 | −8.3 | −8.1 | −8.2 | −8.5 | −6.8 |
| 4 | −7.9 | −7.7 | −9.3 | −8.2 | −9.1 | −8.5 | −8.7 | −9.3 | −7.6 |
| 5 | −9.2 | −8.2 | −10.3 | −9.5 | −10.2 | −9.9 | −9.4 | −10.3 | −8.5 |
| 6 | −8.5 | −8.6 | −10.3 | −9.6 | −9.6 | −9.1 | −9.4 | −9.7 | −8.9 |
| 7 | −9 | −9 | −11.2 | −10.3 | −9.7 | −9.4 | −8.9 | −9.6 | −9.2 |
| 8 | −7.7 | −7.7 | −10.1 | −8.2 | −10.4 | −9.2 | −9.9 | −10.5 | −8.9 |
| 9 | −8.1 | −8.6 | −10.5 | −10.2 | −9.9 | −9.6 | −9.7 | −10 | −9.1 |
| 10 | −8.4 | −8.2 | −9 | −8.7 | −9.9 | −9.1 | −9.3 | −9.9 | −8.2 |
| 11 | −7.5 | −7.6 | −8.7 | −8 | −9 | −8.8 | −8.9 | −9.3 | −7.5 |
| 12 | −7.7 | −7.5 | −9.1 | −8.5 | −9.7 | −9.1 | −9.2 | −10 | −7.6 |
| 13 | −7.9 | −7.7 | −9.4 | −9 | −9.7 | −9.5 | −9.5 | −9.7 | −7.9 |
| 14 | −8.3 | −7.5 | −9.2 | −8.6 | −9.7 | −9 | −9.3 | −9.7 | −8.4 |
| 15 | −7.8 | −7.3 | −9.3 | −8.6 | −9.1 | −9.3 | −9.4 | −9.3 | −7.8 |
| 16 | −7.8 | −7.3 | −9.4 | −8.5 | −9.3 | −8.8 | −8.7 | −9.5 | −8 |
| 17 | −8.2 | −7.4 | −9.1 | −8.6 | −9.9 | −9.4 | −9.1 | −10 | −8.3 |
| 18 | −9.1 | −8.1 | −9.9 | −9.2 | −10.9 | −9.8 | −10.4 | −11 | −8.9 |
| 19 | −9.3 | −8.8 | −10.2 | −9.4 | −10.3 | −9.6 | −9.2 | −10.5 | −9.1 |
| 20 | −8.7 | −7.9 | −9.7 | −8.9 | −10.4 | −9.6 | −9.8 | −10.7 | −8.6 |
| 21 | −9.3 | −9 | −10.4 | −9.7 | −10.4 | −9.1 | −9.5 | −10.2 | −9.7 |
| 22 | −9.2 | −8.8 | −10.2 | −9.7 | −9.6 | −9.1 | −9.4 | −10.1 | −8.8 |
| 23 | −9.7 | −8.9 | −10.3 | −9.7 | −9.5 | −9 | −9.4 | −9.7 | −9.2 |
| | Docked protein¹ | | | | | | | | |
| | 55 | 56 | 50 | 51 | 56 | 57 | 59 | 60 | 62 |
| Compound | ΔG (kcal/mol) | | | | | | | | |
| 1 | −8.5 | −6.3 | −4.9 | −7.9 | −7 | −5.5 | −7.7 | −7.3 | −6.6 |
| 2 | −8.5 | −6.4 | −5 | −8.5 | −7 | −5.7 | −7.8 | −7.7 | −6.8 |
| 3 | −8.3 | −6.3 | −4.3 | −7.4 | −6.8 | −5.6 | −7.7 | −7 | −6.7 |
| 4 | −8.8 | −6.6 | −5.7 | −8.9 | −7.2 | −5.8 | −7.8 | −7.5 | −6.9 |
| 5 | −10 | −6.6 | −7.6 | −8 | −7.8 | −6.7 | −7.9 | −8.6 | −8.3 |
| 6 | −9.9 | −7.1 | −7 | −9.1 | −7.6 | −6.5 | −8.7 | −8.2 | −8.4 |
| 7 | −11.4 | −6.5 | −7 | −8 | −8.3 | −6.9 | −8.6 | −8.4 | −9 |
| 8 | −9.2 | −7.6 | −7.3 | −9.7 | −8.4 | −6.5 | −8.5 | −9.1 | −8.5 |
| 9 | −10.6 | −7.1 | −7.4 | −8.5 | −8.1 | −7 | −8.6 | −8.4 | −9.1 |
| 10 | −9.6 | −6.8 | −5.4 | −7.8 | −7.7 | −5.8 | −8 | −7.5 | −7.4 |
| 11 | −9.1 | −6.5 | −5.8 | −8.4 | −6.6 | −5.9 | −7.4 | −7.1 | −7.2 |
| 12 | −9.5 | −6.3 | −6.1 | −7.7 | −6.9 | −6 | −7.7 | −7.4 | −7.6 |
| 13 | −9.9 | −6.6 | −6.4 | −8 | −7.4 | −6.2 | −7.6 | −7.7 | −7.9 |
| 14 | −9.5 | −7 | −6.3 | −8.6 | −7.2 | −6.3 | −8.2 | −8.4 | −7.9 |
| 15 | −9.6 | −6.7 | −6.5 | −8.5 | −7.1 | −6.1 | −7.9 | −8.4 | −7.2 |
| 16 | −9.6 | −6.9 | −4.5 | −7.7 | −7.1 | −6.6 | −7.2 | −7.5 | −7.6 |
| 17 | −9.4 | −6.7 | −5.4 | −8.2 | −7.2 | −6.2 | −8.4 | −8.2 | −7.8 |
| 18 | −10.6 | −7.4 | −3.2 | −9.3 | −8.1 | −7.2 | −8.4 | −8 | −8.4 |
| 19 | −11.3 | −7 | −5.9 | −7.9 | −7.9 | −7.4 | −9 | −8 | −8.8 |
| 20 | −10.5 | −7.3 | −4.6 | −8.2 | −8.1 | −6.9 | −9.2 | −7.8 | −8.1 |
| 21 | −11.5 | −7 | −4.5 | −8.5 | −8 | −7.7 | −8.7 | −8.4 | −9 |
| 22 | −10.8 | −6.6 | −6.3 | −7.8 | −7.7 | −7.2 | −8.5 | −7.7 | −8.5 |
| 23 | −11.3 | −6.7 | −6.8 | −7.3 | −7.8 | −7.7 | −8.7 | −7.9 | −8.6 |

Docked protein¹

| | | | |
|---|---|---|---|
| 63 | 64 | 65 | 40 |

| | |
|---|---|
| Compound | ΔG (kcal/mol) |

| | | | | |
|---|---|---|---|---|
| 1 | −8.2 | −8.2 | −3.3 | −6.6 |
| 2 | −8.1 | −7.8 | −4.3 | −7 |
| 3 | −8.3 | −7.8 | −3 | −6.7 |
| 4 | −8.5 | −7.9 | −5.1 | −7 |
| 5 | −9.7 | −9.3 | −3.9 | −9.2 |
| 6 | −9.8 | −9.4 | −4.4 | −8.2 |
| 7 | −10.5 | −10.5 | −2.7 | −8.1 |
| 8 | −9.6 | −9.9 | −4.6 | −8 |
| 9 | −10.1 | −9.8 | −4 | −7.7 |
| 10 | −9.1 | −8.1 | −3 | −7.4 |
| 11 | −8.6 | −8.1 | −3.7 | −7.2 |
| 12 | −9 | −8.3 | −3.4 | −7.5 |
| 13 | −9.6 | −8.6 | −3.6 | −7.3 |
| 14 | −9.3 | −9 | −2.4 | −7.2 |
| 15 | −9 | −8.6 | −3.3 | −7.2 |
| 16 | −9.8 | −8.3 | −3.7 | −7 |
| 17 | −9 | −8.8 | −2.5 | −7.5 |
| 18 | −10 | −9.5 | −4.2 | −7.8 |
| 19 | −10.1 | −10 | −3.3 | −7.9 |
| 20 | −9.9 | −9.7 | −3 | −6.5 |
| 21 | −10.5 | −10.2 | −4 | −8.3 |
| 22 | −9.7 | −9.9 | −4.7 | −7.9 |
| 23 | −10.3 | −10.4 | −1.7 | −8.5 |

¹The number of the docked protein corresponds to the entry number found in Table 1.

The compound with strongest binding affinity for dopamine transporter is (7R,12bR)-7,8,9,10-tetrafluoro-1H,2H,3H,4H,6H,7H,12H,12bH-indolo[2,3-a]quinolizin-4-one (40).

The compound with strongest binding affinity for dopamine 1 receptor is (5S,6S,10bS)-6,9-difluoro-5-methyl-1,5,6,10b-tetrahydropyrrolo[2,1-a]isoquinolin-3(2H)-one (25).

The compound with strongest binding affinity for dopamine 2 receptor is (5R,6S,10bR)-6,8,9-trifluoro-5-methyl-1,5,6,10b-tetrahydropyrrolo[2,1-a]isoquinolin-3(2H)-one (26).

The compound with strongest binding affinity for dopamine 3 receptor is (5S,10R,10aR)-7,8,10-trifluoro-5-methyl-1,5,10,10a-tetrahydropyrrolo[1,2-b]isoquinolin-3(2H)-one (42).

The compound with strongest binding affinity for dopamine 4 receptor is (7R,12bS)-7,8,9,10-tetrafluoro-1H,2H,3H,4H,6H,7H,12H,12bH-indolo[2,3-a]quinolizin-4-one (39).

The compound with strongest binding affinity for norepinephrine transporter is (12bS)-8,9-difluoro-1H,2H,3H,4H,6H,7H,12H,12bH-indolo[2,3-a]quinolizin-4-one (27).

The compound with strongest binding affinity for phenylethanolamine N-methyltransferase is 7,8-difluoro-5-methyl-1,5,10,10a-tetrahydropyrrolo[1,2-b]isoquinolin-3(2H)-one (17), preferably the (5S,10aR)-7,8-difluoro-5-methyl-1,5,10,10a-tetrahydropyrrolo[1,2-b]isoquinolin-3(2H)-one isomer.

The compound with strongest binding affinity for serotonin transporter is (7S,12bS)-7,8,9-trifluoro-1H,2H,3H,4H,6H,7H,12H,12bH-indolo[2,3-a]quinolizin-4-one (30).

The compound with strongest binding affinity for serotonin N-acetyltransferase is (7R,12bS)-7,8,9,10-tetrafluoro-1H,2H,3H,4H,6H,7H,12H,12bH-indolo[2,3-a]quinolizin-4-one (39).

The compound with strongest binding affinity for N-acetylserotonin methyltransferase is (5S,10R,10aS)-7,8,10-trifluoro-5-methyl-1,5,10,10a-tetrahydropyrrolo[1,2-b]isoquinolin-3(2H)-one (43).

The compound with strongest binding affinity for 5-HT1A receptor is (7R,12bR)-7,8,9-trifluoro-1H,2H,3H,4H,6H,7H,12H,12bH-indolo[2,3-a]quinolizin-4-one (29).

The compound with strongest binding affinity for 5-HT2A receptor is 7,8,9,10-tetrafluoro-1H,2H,3H,4H,6H,7H,12H,12bH-indolo[2,3-a]quinolizin-4-one (7), preferably the (7S,12bS)-7,8,9,10-tetrafluoro-1H,2H,3H,4H,6H,7H,12H,12bH-indolo[2,3-a]quinolizin-4-one isomer.

The compound with strongest binding affinity for 5-HT1B receptor is (12bS)-8,9-difluoro-1H,2H,3H,4H,6H,7H,12H,12bH-indolo[2,3-a]quinolizin-4-one (27).

The compound with strongest binding affinity for 5-HT2B receptor is (7R,12bS)-7,8,9-trifluoro-1H,2H,3H,4H,6H,7H,12H,12bH-indolo[2,3-a]quinolizin-4-one (31).

The compound with strongest binding affinity for 5-HT2C receptor is (5S,10S,10aR)-7,8,10-trifluoro-5-methyl-1,5,10,10a-tetrahydropyrrolo[1,2-b]isoquinolin-3(2H)-one (44).

The compound with strongest binding affinity for 5-HT3 receptor is (4S)-4,6-difluoro-1-methyl-4,9-dihydro-3H-pyrido[3,4-b]indole (24).

The compound with strongest binding affinity for monoamino-oxidase A is 7,10-difluoro-5-methyl-1,5,10,10a-tetrahydropyrrolo[1,2-b]isoquinolin-3(2H)-one (15), preferably the (5R,10R,10aS)-7,10-difluoro-5-methyl-1,5,10,10a-tetrahydropyrrolo[1,2-b]isoquinolin-3(2H)-one isomer.

The compound with strongest binding affinity for monoamino-oxidase B is (1S,4S)-4,6-difluoro-1-methyl-2,3,4,9-tetrahydro-1H-pyrido[3,4-b]indole (4).

The compound with strongest binding affinity for catechol-O-methyltransferase is 7,8,9-trifluoro-1H,2H,3H,4H,6H,7H,12H,12bH-indolo[2,3-a]quinolizin-4-one (28), preferably the (7S,12bR)-7,8,9-trifluoro-1H,2H,3H,4H,6H,7H,12H,12bH-indolo[2,3-a]quinolizin-4-one isomer.

The compound with strongest binding affinity for alpha-1A adrenergic receptor is (7S,12bR)-7,8,9,10-tetrafluoro-1H,2H,3H,4H,6H,7H,12H,12bH-indolo[2,3-a]quinolizin-4-one (43).

The compound with strongest binding affinity for alpha-1B adrenergic receptor is (12bS)-9,10-difluoro-1H,2H,3H,4H,6H,7H,12H,12bH-indolo[2,3-a]quinolizin-4-one (45).

The compound with strongest binding affinity for alpha-2A adrenergic receptor is (12bS)-8,9-difluoro-1H,2H,3H,4H,6H,7H,12H,12bH-indolo[2,3-a]quinolizin-4-one (27).

The compound with strongest binding affinity for alpha-2B adrenergic receptor is (5S,6R,10bS)-6,8-difluoro-5-methyl-1,5,6,10b-tetrahydropyrrolo[2,1-a]isoquinolin-3(2H)-one (32).

The compound with strongest binding affinity for alpha-2C adrenergic receptor is 5,6,7-trifluoro-1-methyl-4,9-dihydro-3H-pyrido[3,4-b]indole (12).

The compound with strongest binding affinity for beta-1 adrenergic receptor is (5S,10S,10aR)-8,10-difluoro-5-methyl-1,5,10,10a-tetrahydropyrrolo[1,2-b]isoquinolin-3(2H)-one (33).

The compound with strongest binding affinity for beta-2 adrenergic receptor is (1R,4R)-4,6,7-trifluoro-1-methyl-2,3,4,9-tetrahydro-1H-pyrido[3,4-b]indole (34).

The compound with strongest binding affinity for phenylalanine hydroxylase is (7R,12bS)-7,9,10-trifluoro-1H,2H,3H,4H,6H,7H,12H,12bH-indolo[2,3-a]quinolizin-4-one (35).

The compound with strongest binding affinity for tyrosine hydroxylase is (75,12bS)-7,8,9-trifluoro-1H,2H,3H,4H,6H,7H,12H,12bH-indolo[2,3-a]quinolizin-4-one (30).

The compound with strongest binding affinity for H1 histamine receptor is (7R,12bR)-7,8-difluoro-1H,2H,3H,4H,6H,7H,12H,12bH-indolo[2,3-a]quinolizin-4-one (36).

The compound with strongest binding affinity for H3 histamine receptor is (7S,12bR)-7,8,9,10-tetrafluoro-1H,2H,3H,4H,6H,7H,12H,12bH-indolo[2,3-a]quinolizin-4-one (41).

The compound with strongest binding affinity for OX1 orexin receptor is (7R,12bS)-7,8,9-trifluoro-1H,2H,3H,4H,6H,7H,12H,12bH-indolo[2,3-a]quinolizin-4-one (31).

The compound with strongest binding affinity for OX2 orexin receptor is (7R,12bS)-7,8,9-trifluoro-1H,2H,3H,4H,6H,7H,12H,12bH-indolo[2,3-a]quinolizin-4-one (31).

The compound with strongest binding affinity for prolactin receptor is (7R,12bR)-7,8,9-trifluoro-1H,2H,3H,4H,6H,7H,12H,12bH-indolo[2,3-a]quinolizin-4-one (29).

The compound with strongest binding affinity for NMDA receptor GluN1 is (5R,6R,10bS)-6,9-difluoro-5-methyl-1,5,6,10b-tetrahydropyrrolo[2,1-a]isoquinolin-3(2H)-one (37).

The compound with strongest binding affinity for NMDA receptor Glu2B is 4,5,6,7-tetrafluoro-1-methyl-2,3,4,9-tetrahydro-1H-pyrido[3,4-b]indole (10), preferably the (1S,4R)-4,5,6,7-tetrafluoro-1-methyl-2,3,4,9-tetrahydro-1H-pyrido[3,4-b]indole isomer.

The compound with strongest binding affinity for alpha-4-beta-2 nicotinic receptor is 6-fluoro-1-methyl-4,9-dihydro-3H-pyrido[3,4-b]indole (38).

The compound with strongest binding affinity for sigma-1 receptor is (7S,12bS)-7,8,9-trifluoro-1H,2H,3H,4H,6H,7H,12H,12bH-indolo[2,3-a]quinolizin-4-one (30).

The compound with strongest binding affinity for nociceptin/orphanin receptor is (7R,12bR)-7,8,9,10-tetrafluoro-1H,2H,3H,4H,6H,7H,12H,12bH-indolo[2,3-a]quinolizin-4-one (40).

The compound with strongest binding affinity for mu-opioid receptor is (7S,12bR)-7,8,9,10-tetrafluoro-1H,2H,3H,4H,6H,7H,12H,12bH-indolo[2,3-a]quinolizin-4-one (41).

The compound with strongest binding affinity for delta-opioid receptor is 7,8,9,10-tetrafluoro-1H,2H,3H,4H,6H,7H,12H,12bH-indolo[2,3-a]quinolizin-4-one (7), preferably the (7S,12bS)-7,8,9,10-tetrafluoro-1H,2H,3H,4H,6H,7H,12H,12bH-indolo[2,3-a]quinolizin-4-one isomer.

It will be obvious to a person skilled in the art that, as the technology advances, the inventive concept can be implemented in various ways. The invention and its embodiments are not limited to the examples described above but may vary within the scope of the claims.

1. A compound of formula (I) 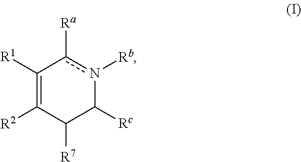
wherein the dotted line represents an optional bond;
R¹ and R², together with the carbon atoms they are attached to, form a group selected from a 1H-indole group and a benzene group, and said 1H-indole group and benzene group being optionally substituted with one to four substituent(s) each independently selected from the group consisting of R³, R⁴, R⁵, and R⁶, wherein each R³, R⁴, R⁵, and R⁶ is independently selected from the group consisting of halogen, OH, C₁₋₄-alkyl, C₁₋₃-(per)haloalkyl, C₁₋₃-alkoxy, C₁₋₃-(per)haloalkoxy;
R^{a} and R^{b}, together with the carbon atom and nitrogen atom they are attached to, form a group selected from a 5- and 6-membered cyclic amide, and
R^{c} is H; or
R^{a} is Me, and
R^{b} and R^{c}, together with the nitrogen atom and carbon atom to which R^{b} and R^{c} are attached, form a group selected from a 5- and 6-membered cyclic amide; or
R^{a} is Me,
R^{b} is H, or R^{b} is absent when the dotted line represents a bond, and
R^{c} is H, provided that R¹ and R², together with the carbon atoms that R¹ and R² are attached to, form said optionally substituted 1H-indole group;
R⁷ is selected from the group consisting of halogen, OH, oxo, SH, NOR⁸, C₁₋₃-(per)haloalkyl, C₁₋₃-alkoxy, C₁₋₃-(per)haloalkoxy, CN, C(O)N(R⁸)₂, and N(R⁸)₂, or
R⁷ may also be C₁₋₄-alkyl with the provisio that said 1H-indole group or benzene group is substituted with one to four substituent(s) each independently selected from the group consisting of halogen, OH, C₁₋₄-alkyl, C₁₋₃-(per)haloalkyl, C₁₋₃-alkoxy, C₁₋₃-(per)haloalkoxy, or
R⁷ may also be H provided that
when R^{a} and R^{b}, together with the carbon atom and nitrogen atom they are attached to, form a group selected from a 5- and 6-membered cyclic amide, R^{c} is H, then R⁴ and R⁵ is each independently selected from the group consisting of halogen, C₁₋₄-alkyl, C₁₋₃-(per)haloalkyl, and C₁₋₃-(per)haloalkoxy, or
when R^{a} is Me, R^{b} and R^{c}, together with the carbon atom and nitrogen atom they are attached to, form a 6-membered cyclic amide, then R¹ and R², together with the carbon atoms they are attached to, form said optionally substituted 1H-indole group, or
when R^{a} is Me, R^{b} and R^{c}, together with the carbon atom and nitrogen atom they are attached to, form a 5-membered cyclic amide, and R¹ and R², together with the carbon atoms they are attached to, form a 1H-indole group or a benzene group, then said 1H-indole group or benzene group is substituted with one to four substituent(s) each independently selected from the group consisting of halogen, OH, C₁₋₄-alkyl, C₁₋₃-(per)haloalkyl, C₁₋₃-alkoxy, and C₁₋₃-(per)haloalkoxy, or
when R^{a} is Me, R^{c} is H, R¹ and R², together with the carbon atoms they are attached to, form a substituted 1H-indole group, then R⁴ and R⁵ of said substituted 1H-indole group is each independently selected from the group consisting of halogen, C₁₋₄-alkyl, C₁₋₃-(per)haloalkyl, and C₁₋₃-(per)haloalkoxy;
each R⁸ is independently selected from the group consisting of H, C₁₋₄-alkyl, C₁₋₄-alkenyl, C₁₋₄-alkynyl, and C₁₋₃-(per)haloalkyl, or when part of any N(R⁸)₂ both R⁸ together with the nitrogen they are attached to may form a 3- to 6-membered aliphatic or aromatic heterocyclic ring comprising 1 to 3 heteroatoms each independently selected from N, O, and S;
or a stereoisomer or a pharmaceutically acceptable salt thereof.

2. A compound as claimed in claim 1, wherein the compound has formula (Ia), (Ib), or (Ic), 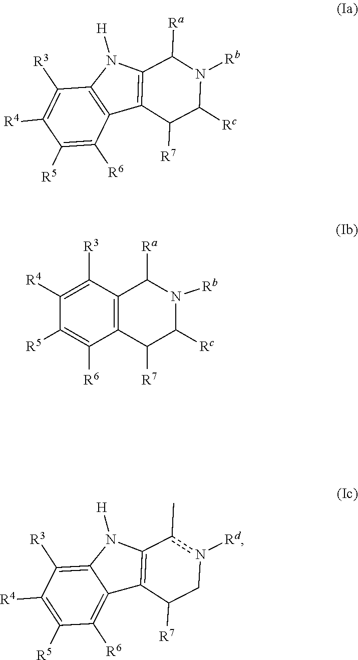
wherein
R^{a} and R^{b}, together with the carbon atom and nitrogen atom they are attached to, form a group selected from a 5- and 6-membered cyclic amide, and
R^{c} is H; or
R^{a} is Me, and
R^{b} and R^{c}, together with the nitrogen atom and carbon atom they are attached to, form a group selected from a 5- and 6-membered cyclic amide;
R^{d} is H, or R^{d} is absent when the dotted line represents a bond; and
R³, R⁴, R⁵, R⁶, R⁷, R⁸, and the dotted line are as defined in claim 1;
or a stereoisomer or a pharmaceutically acceptable salt thereof.

3. A compound as claimed in claim 1, wherein the compound has formula (Ic), (Id), (Ie), (If), or (Ig), 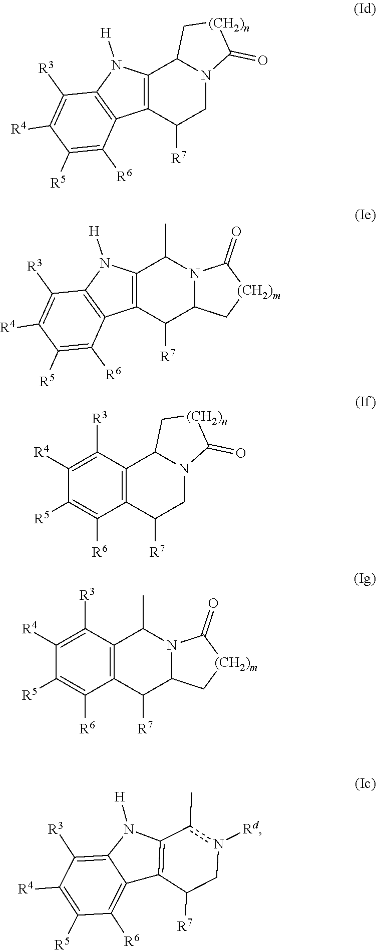
wherein
m is 1 or 2;
n is 1 or 2;
R^{d} is H, or R^{d} is absent when the dotted line represents a bond; and
the dotted line, R³, R⁴, R⁵, R⁶, R⁷, and R⁸ are as defined in claim 1;
or a stereoisomer or a pharmaceutically acceptable salt thereof.

4. A compound as claimed in claim 2, wherein the compound has formula (Id), (Ie), (If), or (Ig), wherein
m is 1 or 2;
n is 1 or 2; and
R³, R⁴, R⁵, R⁶, R⁷, and R⁸ are as defined in claim 1;
or a stereoisomer or a pharmaceutically acceptable salt thereof.

5. A compound as claimed in claim 2, wherein the compound has formula (Ic), wherein
R^{d} is H, or R^{d} is absent when the dotted line represents a bond; and
the dotted line, R³, R⁴, R⁵, R⁶, R⁷, and R⁸ are as defined in claim 1;
or a stereoisomer or a pharmaceutically acceptable salt thereof.

6. A compound as claimed in claim 1, wherein
R³ and R⁶ are each independently selected from the group consisting of H, halogen, C₁₋₄-alkyl, C₁₋₃-(per)haloalkyl, and C₁₋₃-(per)haloalkoxy;
R⁴ and R⁵ are both halogen, or one of R⁴ and R⁵ is halogen and the other is C₁₋₄-alkyl, C₁₋₃-(per)haloalkyl, or C₁₋₃-(per)haloalkoxy; and
R⁷ is selected from the group consisting of H, halogen, OH, oxo, NOR⁸, C₁₋₄-alkyl, C₁₋₃-(per)haloalkyl, C₁₋₃-alkoxy, and C₁₋₃-(per)haloalkoxy;
or a stereoisomer or a pharmaceutically acceptable salt thereof.

7. A compound as claimed in claim 1, wherein
R³ and R⁶ are each independently selected from the group consisting of H, halogen, C₁₋₄-alkyl, C₁₋₃-(per)haloalkyl, and C₁₋₃-(per)haloalkoxy;
R⁴ and R⁵ are both F, or one of R⁴ and R⁵ is F and the other is C₁₋₄-alkyl or C₁₋₃-(per)haloalkyl; and
R⁷ is selected from the group consisting of H, halogen, OH, oxo, NOR⁸, C₁₋₄-alkyl, C₁₋₃-(per)haloalkyl, C₁₋₃-alkoxy, and C₁₋₃-(per)haloalkoxy;
or a stereoisomer or a pharmaceutically acceptable salt thereof.

8. A compound as claimed in claim 1, wherein
R³ and R⁶ are each independently selected from H and halogen;
R⁴ and R⁵ are both F, or one of R⁴ and R⁵ is F and the other is C₁₋₄-alkyl or C₁₋₃-(per)haloalkyl; and
R⁷ is selected from the group consisting of H, F, OH, C₁₋₄-alkyl, and methoxy;
or a stereoisomer or a pharmaceutically acceptable salt thereof.

9. A compound as claimed in claim 1, wherein
R³, R⁶, and R⁷ are H; and
R⁴ and R⁵ are F;
or a stereoisomer or a pharmaceutically acceptable salt thereof.

10. A compound as claimed in claim 1, wherein
R³ and R⁶ are H; and
R⁴, R⁵ and R⁷ are F;
or a stereoisomer or a pharmaceutically acceptable salt thereof.

11. A compound as claimed in claim 10, wherein
R^{a} is Me; and
R^{b} and R^{c}, together with the nitrogen atom and carbon atom they are attached to, form a group selected from a 5- and 6-membered cyclic amide;
or a stereoisomer or a pharmaceutically acceptable salt thereof.

12. A compound as claimed in claim 1, wherein
R³ and R⁶ are each independently selected from the group consisting of H, halogen, C₁₋₄-alkyl, C₁₋₃-(per)haloalkyl, and C₁₋₃-(per)haloalkoxy;
R⁴ and R⁵ are both F, or one of R⁴ and R⁵ is halogen and the other is H, C₁₋₄-alkyl or C₁₋₃-(per)haloalkyl; and
R⁷ is selected from the group consisting of halogen, OH, C₁₋₃-alkoxy, C₁₋₃-(per)haloalkoxy;
or a stereoisomer or a pharmaceutically acceptable salt thereof.

13. A compound as claimed in claim 1, wherein
R³ and R⁶ are each independently selected from the group consisting of H and F;
R⁴ and R⁵ are both F, or one of R⁴ and R⁵ is F and the other is H, C₁₋₄-alkyl or C₁₋₃-(per)haloalkyl; and
R⁷ is selected from the group consisting of F, OH, methoxy, and ethoxy;
or a stereoisomer or a pharmaceutically acceptable salt thereof.

14. A compound as claimed in claim 1, wherein the compound is selected from a group consisting of:
5-Fluoro-1-methyl-2,3,4,9-tetrahydro-1H-pyrido[3,4-b]indole;
6-Fluoro-1-methyl-2,3,4,9-tetrahydro-1H-pyrido[3,4-b]indole;
4,6-Difluoro-1-methyl-2,3,4,9-tetrahydro-1H-pyrido[3,4-b]indole;
7-Fluoro-1H,2H,3H,4H,6H, 7H, 12H, 12bH-indolo[2,3-a]quinolizin-4-one;
7,8,9,10-Tetrafluoro-1H,2H,3H,4H,6H,7H, 12H, 12bH-indolo[2,3-a]quinolizin-4-one;
(7R,12bS)-7,8,9,10-Tetrafluoro-1H,2H,3H,4H,6H,7H, 12H, 12bH-indolo[2,3-a]quinolizin-4-one;
(7R,12bR)-7,8,9,10-Tetrafluoro-1H,2H,3H,4H,6H,7H, 12H, 12bH-indolo[2,3-a]quinolizin-4-one;
(7S,12bR)-7,8,9,10-Tetrafluoro-1H,2H,3H,4H,6H, 7H, 12H, 12bH-indolo[2,3-a]quinolizin-4-one;
7,9-Difluoro-1H,2H,3H,4H,6H,7H, 12H, 12bH-indolo[2,3-a]quinolizin-4-one;
7,8,10-Trifluoro-1H,2H,3H,4H,6H, 7H, 12H, 12bH-indolo[2,3-a]quinolizin-4-one;
4,5,6,7-Tetrafluoro-1-methyl-2,3,4,9-tetrahydro-1H-pyrido[3,4-b]indole;
5,6-difluoro-1-methyl-4,9-dihydro-3H-pyrido[3,4-b]indole;
5,6,7-trifluoro-1-methyl-4,9-dihydro-3H-pyrido[3,4-b]indole;
4,5,6,7-tetrafluoro-1-methyl-4,9-dihydro-3H-pyrido[3,4-b]indole;
7,8,10-Trifluoro-5-methyl-1,5,10,10a-tetrahydropyrrolo[1,2-b]isoquinolin-3(2H)-one;
(5S,10R,10aR)-7,8,10-Trifluoro-5-methyl-1,5,10,10a-tetrahydropyrrolo[1,2-b]isoquinolin-3(2H)-one;
(5S,10R,10aS)-7,8,10-Trifluoro-5-methyl-1,5,10,10a-tetrahydropyrrolo[1,2-b]isoquinolin-3(2H)-one;
(5S,10S,10aR)-7,8,10-Trifluoro-5-methyl-1,5,10,10a-tetrahydropyrrolo[1,2-b]isoquinolin-3(2H)-one;
7,10-Difluoro-5-methyl-1,5,10,10a-tetrahydropyrrolo[1,2-b]isoquinolin-3(2H)-one;
9,10-Difluoro-5-methyl-1,5,10,10a-tetrahydropyrrolo[1,2-b]isoquinolin-3(2H)-one;
7,8-Difluoro-5-methyl-1,5,10,10a-tetrahydropyrrolo[1,2-b]isoquinolin-3(2H)-one;
(5R,11S)-10,11-Difluoro-5-methyl-1,2,5,6,11,11a-hexahydro-3H-indolizino[6,7-b]indol-3-one;
(6R,12S)-12-Fluoro-6-methyl-6,9,10,11,11a, 12-hexahydroindolo[3,2-b]quinolizin-8(5H)-one;
(5R,11S)-11-Fluoro-5-methyl-1,2,5,6,11,11a-hexahydro-3H-indolizino[6,7-b]indol-3-one;
(6R,12S)-1,12-Difluoro-6-methyl-6,9,10,11,11a, 12-hexahydroindolo[3,2-b]quinolizin-8(5H)-one;
(5R)-9-Fluoro-5-methyl-1,2,5,6,11,11a-hexahydro-3H-indolizino[6,7-b]indol-3-one;
(6R)-2-Fluoro-6-methyl-6,9,10,11,11a, 12-hexahydroindolo[3,2-b]quinolizin-8(5H)-one;
(4S)-4,6-difluoro-1-methyl-4,9-dihydro-3H-pyrido[3,4-b]indole;
(5S,6S,10bS)-6,9-difluoro-5-methyl-1,5,6,10b-tetrahydropyrrolo[2,1-a]isoquinolin-3(2H)-one;
(5R,6R,10bS)-6,9-difluoro-5-methyl-1,5,6,10b-tetrahydropyrrolo[2,1-a]isoquinolin-3(2H)-one;
(5R,6S,10bR)-6,8,9-trifluoro-5-methyl-1,5,6,10b-tetrahydropyrrolo[2,1-a]isoquinolin-3(2H)-one;
(12bS)-8,9-difluoro-1H,2H,3H,4H,6H,7H, 12H, 12bH-indolo[2,3-a]quinolizin-4-one;
(7R,12bS)-7,8,9-trifluoro-1H,2H,3H,4H,6H,7H, 12H, 12bH-indolo[2,3-a]quinolizin-4-one;
(7S,12bR)-7,8,9-trifluoro-1H,2H,3H,4H,6H,7H,12H, 12bH-indolo[2,3-a]quinolizin-4-one;
(7R,12bR)-7,8,9-trifluoro-1H,2H,3H,4H,6H,7H, 12H, 12bH-indolo[2,3-a]quinolizin-4-one;
(12bS)-9,10-difluoro-1H,2H,3H,4H,6H,7H, 12H, 12bH-indolo[2,3-a]quinolizin-4-one;
(7R,12bR)-7,8,9-trifluoro-1H,2H,3H,4H,6H, 7H, 12H, 12bH-indolo[2,3-a]quinolizin-4-one;
(7S,12bS)-7,8,9-trifluoro-1H,2H,3H,4H,6H,7H, 12H, 12bH-indolo[2,3-a]quinolizin-4-one;
(5S,6R,10bS)-6,8-difluoro-5-methyl-1,5,6,10b-tetrahydropyrrolo[2,1-a]isoquinolin-3(2H)-one;
(5S,10S,10aR)-8,10-difluoro-5-methyl-1,5,10,10a-tetrahydropyrrolo[1,2-b]isoquinolin-3(2H)-one;
(1R,4R)-4,6,7-trifluoro-1-methyl-2,3,4,9-tetrahydro-1H-pyrido[3,4-b]indole;
(7R,12bS)-7,9,10-trifluoro-1H,2H,3H,4H,6H,7H, 12H, 12bH-indolo[2,3-a]quinolizin-4-one;
(7R,12bR)-7,8-difluoro-1H,2H,3H,4H,6H,7H, 12H, 12bH-indolo[2,3-a]quinolizin-4-one;
(5R,6R,10bS)-6,9-difluoro-5-methyl-1,5,6,10b-tetrahydropyrrolo[2,1-a]isoquinolin-3(2H)-one; and
6-fluoro-1-methyl-4,9-dihydro-3H-pyrido[3,4-b]indole;
or a stereoisomer or a pharmaceutically acceptable salt thereof.

15. A pharmaceutical composition comprising an effective amount of one or more compounds, or a stereoisomer or a pharmaceutically acceptable salt thereof, as claimed in claim 1, together with one or more pharmaceutically acceptable excipient(s).

16. The pharmaceutical composition as claimed in claim 15 comprising one or more compounds, or a stereoisomer or a pharmaceutically acceptable salt thereof, as claimed in claim 1, in combination with one or more pharmaceutically acceptable carrier(s).

17. The pharmaceutical composition as claimed in claim 15 comprising one or more compounds, or a stereoisomer or a pharmaceutically acceptable salt thereof, in combination with one or more other active ingredient(s).

18. A compound, or a stereoisomer or pharmaceutically acceptable salt thereof, as claimed in claim 1, for use as a medicament.

19. A compound, or a stereoisomer or pharmaceutically acceptable salt thereof, as claimed in claim 1, for use in treatment or prevention of CNS related diseases or conditions.

20. A compound, or a stereoisomer or pharmaceutically acceptable salt thereof, as claimed in claim 1, for use in the treatment or prevention of a disease or condition selected from the group consisting of Alzheimer's disease, Parkinson's disease, depression, anxiety, hyperactivity, narcolepsy, drug addiction, alcoholism, anorexia, bulimia, and mitochondrial disease.

21. A method for the preparation of a compound of formula (I), or pharmaceutically acceptable salt or a stereoisomer thereof, as defined in claim 1, comprising steps of:
providing a compound of formula (I′) 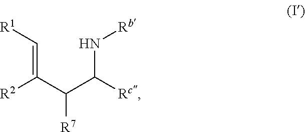
wherein R¹, R², and R⁷ are as defined in claim 1,
wherein when R¹ and R², together with the carbon atoms they are attached to, form a 1H-indole group, then the nitrogen of said 1H-indole group is optionally protected with a protecting group,
wherein when R⁷ is OH or SH, then the oxygen or sulphur of said OH or SH is optionally protected with a protecting group,
R^{b′} and R^{c″}, together with the carbon atom and nitrogen atom they are attached to, form a group selected from a 5- and 6-membered cyclic amide, or
R^{b′} is H or activating group, and R^{c″} is H;
reacting said compound of formula (I′) with an aldehyde, optionally in the presence of one or more activating group reactant(s), which/that optionally together with one or more activating agent(s) facilitate(s) a ring formation;
optionally performing one or more deprotection reaction(s);
to obtain the compound of formula (I) 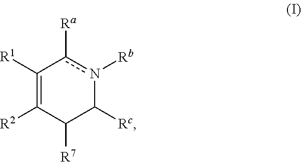
wherein R¹, R², R⁷, R^{a}, R^{b}, R^{c}, and the dotted line are as defined in claim 1; and
optionally converting the compound of formula (I) to a pharmaceutically acceptable salt thereof.
